(19) United States
(12) Patent Application Publication
(10) Pub. No.: US 2024/0374386 A1
(43) Pub. Date: Nov. 14, 2024

(54) DELIVERY DEVICES AND METHODS FOR HEART VALVE REPAIR AND REPLACEMENT DEVICES

(71) Applicant: EDWARDS LIFESCIENCES CORPORATION, Irvine, CA (US)

(72) Inventors: Pablo Hernan Catania, Costa Mesa, CA (US); Munir Hicham Eltal, Irvine, CA (US); Andrew John Akagawa, Lake Forest, CA (US); Robert Bowes, Trabuco Canyon, CA (US); Waina Michelle Chu, Tustin, CA (US); Nicolas Schleiger, Costa Mesa, CA (US); Tam Van Nguyen, Westminster, CA (US); Alena Lian Sim, Tustin, CA (US); Thanh Huyen Tran, Fountain Valley, CA (US); Rupesh Gajanan Nawalakhe, Trabuco Canyon, CA (US); Chris J. Okos, Huntington Beach, CA (US); Eric Robert Dixon, Villa Park, CA (US); Grant Matthew Stearns, Costa Mesa, CA (US); Kevin Gantz, Laguna Beach, CA (US); Daniel Bobo, Santa Ana, CA (US); Aric Daniel Stone, Newport Beach, CA (US)

(21) Appl. No.: 18/784,810

(22) Filed: Jul. 25, 2024

Related U.S. Application Data
(63) Continuation of application No. PCT/US2023/011452, filed on Jan. 24, 2023.
(60) Provisional application No. 63/399,041, filed on Aug. 18, 2022.
(60) Provisional application No. 63/352,635, filed on Jun. 15, 2022.
(60) Provisional application No. 63/267,184, filed on Jan. 26, 2022.

(51) Int. Cl. A61F 2/24

(52) U.S. Cl. CPC A61F 2/2466; A61F 2/246; A61F 2/2463; A61F 2220/0091

(57) ABSTRACT

Delivery systems and methods of delivering devices or implants are disclosed. The devices or implants can be configured as valve repair devices or treatment devices have gripping members that are openable and closable to attach the device to leaflets of a native heart valve. The delivery systems are configured to position the device or implant and open and close the gripping members of the device or implant.

### RELATED PATENT APPLICATIONS

This patent application is a continuation of Patent Cooperation Treat Application No. PCT/US2023/011452, filed on Jan. 24, 2023, which claims priority to U.S. Provisional Application No. 63/267,184, filed on Jan. 26, 2022, U.S. Provisional Application No. 63/352,635, filed Jun. 15, 2022, and U.S. Provisional Application No. 63/399,041, filed Aug. 18, 2022, which are incorporated herein by reference.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

The present disclosure discloses components for a delivery system for a valve repair or replacement device. While not required, these components can make the delivery system easier to use, more ergonomic, more intuitive, and/more accurate than previous delivery systems. One or more of these components can be used with existing delivery systems. Any combination or subcombination of the disclosed components can be used together, but there is no requirement that any of the components disclosed by the present application be used with any other component disclosed by the present application.

In some implementations, devices, such as valve repair devices, have anchors that are openable and closable to attach the device to leaflets of a native heart valve. The anchors have an adjustable width. The delivery systems are configured to position the device, open and close the anchors of the device, and adjust the width of the anchors of the device.

In some implementations, a handle assembly for controlling a transvascular device (e.g., an implantable device, a treatment device, a repair device, etc.) includes a handle housing, a sheath, an actuation element, a paddle actuation knob (or the like, e.g., button, switch, etc.), a paddle width adjustment element, and a paddle width control knob (or the like, e.g., button, switch, etc.). The sheath extends distally from the handle housing. The actuation element extends through the sheath and is configured to be coupled to the device. The paddle actuation knob is coupled to the actuation element. In some implementations, the paddle actuation knob is rotatable relative to the handle housing. Actuation (e.g., rotation, pressing, sliding, etc.) of the paddle actuation knob causes movement (e.g., one or more of axial movement, rotational movement, translational movement, etc.) of the actuation element with respect to the handle housing and the sheath. In some implementations, the movement of the actuation element causes the device to move between open and closed positions.

In some implementations, the paddle width adjustment element extends through the actuation element. The paddle width adjustment element can be configured to be coupled to at least one of a pair of paddles of the device. The paddle width control knob is coupled to the paddle width adjustment element and is rotatable relative to the handle housing. In some implementations, actuation (e.g., rotation, pressing, sliding, etc.) of the paddle width control knob causes movement (e.g., one or more of axial movement, rotational movement, translational movement, etc.) of the paddle width adjustment element with respect to the handle housing and the sheath. In some implementations, the movement of the paddle width adjustment element causes a width of the at least one of the pair of paddles of the device to be moved from a first width to a second width.

In some implementations, the paddle actuation knob is positioned axially between the handle housing and the paddle width control knob.

In some implementations, a distal end of the paddle actuation knob comprises external threads configured to engage with internal threads of the handle housing.

In some implementations, rotation of the paddle width control knob axially drives a frame that is attached to the actuation element. The paddle width control knob can be rotatable relative to the frame.

In some implementations, rotation of the paddle width control knob axially drives the paddle width control knob and the paddle width adjustment element with respect to the paddle actuation knob and the actuation element.

In some implementations, a pair of clasp actuation lines extend through the sheath and are connected to a pair of clasp control members. Each clasp control member is movable relative to the handle housing to move a clasp between an open configuration and a closed configuration.

In some implementations, each clasp actuation line is coupled to a suture lock extending from a proximal end of the handle housing.

In some implementations, each suture lock is angled with respect to a central axis extending through the handle assembly.

In some implementations, the paddle width control knob is coupled to a planetary gearbox and rotation of the paddle width control knob is effective to cause rotation of the planetary gearbox.

In some implementations, the planetary gearbox comprises at least a ring gear, a pair of planet gears, and an elongated central gear.

In some implementations, the elongated central gear is coupled to the paddle width adjustment element through a rotationally fixed follower such that rotation of the elongated central gear causes axial movement of the rotationally fixed follower, which in turn causes axial movement of the paddle width adjustment element with respect to the housing.

In some implementations, external teeth of the elongated central gear engage with teeth of the pair of planet gears.

In some implementations, a delivery system includes a steerable catheter assembly and a device catheter assembly (e.g., an implant catheter assembly, a control catheter assembly, a device control catheter assembly, etc.). The steerable catheter assembly has a handle and a sheath extending from the handle. The device catheter assembly has a handle and a sheath extendable coaxially through the sheath of the steerable catheter assembly. In some implementations, the handle of the device catheter assembly includes a handle housing, a sheath, an actuation element, a paddle actuation knob (or the like, e.g., button, switch, etc.), a paddle width adjustment element, and a paddle width control knob (or the like, e.g., button, switch, etc.). The sheath extends distally from the handle housing. The actuation element extends through the sheath and is configured to be coupled to a device (e.g., a treatment device, a repair device, an implant, an implantable device, a valve repair device, etc.).

In some implementations, the paddle actuation knob (or the like, e.g., button, switch, etc.) is coupled to (e.g., operatively coupled to) the actuation element. In some implementations, the paddle actuation knob is rotatable relative to the handle housing. Actuation (e.g., rotation, pressing, sliding, etc.) of the paddle actuation knob causes movement (e.g., one or more of axial movement, rotational movement, translational movement, etc.) of the actuation element with respect to the handle housing and the sheath. The movement of the actuation element causes the device to move between open and closed positions. The paddle width adjustment element extends through the actuation element. The paddle width adjustment element is configured to be coupled to at least one of a pair of paddles of the device.

In some implementations, the paddle width control knob (or the like, e.g., button, switch, etc.) is coupled to (e.g., operatively coupled to) the paddle width adjustment element. In some implementations, the paddle width control knob is rotatable relative to the handle housing. Actuation (e.g., rotation, pressing, sliding, etc.) of the paddle width control knob causes movement (e.g., one or more of axial movement, rotational movement, translational movement, etc.) of the paddle width adjustment element with respect to the handle housing and the sheath. The movement of the paddle width adjustment element causes a width of the at least one of the pair of paddles of the device to be moved from a first width to a second width.

In some implementations, the paddle actuation knob is positioned axially between the handle housing and the paddle width control knob.

In some implementations, a distal end of the paddle actuation knob comprises external threads configured to engage with internal threads of the handle housing.

In some implementations, rotation of the paddle width control knob axially drives a frame that is attached to the actuation element. The paddle width control knob can be rotatable relative to the frame.

In some implementations, rotation of the paddle width control knob axially drives the paddle width control knob and the paddle width adjustment element with respect to the paddle actuation knob and the actuation element.

In some implementations, a pair of clasp actuation lines extend through the sheath and are connected to a pair of clasp control members. Each clasp control member is movable (e.g., one or more of axially movable, rotationally movable, etc.) relative to the handle housing to move a clasp between an open configuration and a closed configuration.

In some implementations, each clasp actuation line is coupled to a suture lock extending from a proximal end of the handle housing.

In some implementations, each suture lock is angled with respect to a central axis extending through the handle assembly.

In some implementations, the paddle width control knob is coupled to a planetary gearbox and rotation of the paddle width control knob is effective to cause rotation of the planetary gearbox.

In some implementations, the planetary gearbox comprises at least a ring gear, a pair of planet gears, and an elongated central gear.

In some implementations, the elongated central gear is coupled to the paddle width adjustment element through a rotationally fixed follower such that rotation of the elongated central gear causes axial movement of the rotationally fixed follower, which in turn causes axial movement of the paddle width adjustment element with respect to the housing.

In some implementations, external teeth of the elongated central gear engage with teeth of the pair of planet gears.

In some implementations, a method includes delivering a device (e.g., a treatment device, a repair device, an implant, an implantable device, a valve repair device, etc.). In some implementations, the device is coupled to an actuation element, a paddle width adjustment element, and a sheath. The sheath is advanced to position the device at a delivery site. In some implementations, a paddle actuation knob (or the like, e.g., button, switch, etc.) is actuated (e.g., rotated, pressed, slid, etc.) to cause movement (e.g., one or more of axial movement, rotational movement, translational movement, etc.) of the actuation element to move the device from a closed configuration to an open configuration. In some implementations, a paddle width control knob (or the like, e.g., button, switch, etc.) is actuated (e.g., rotated, pressed, slid etc.) to cause movement (e.g., one or more of axial movement, rotational movement, translational movement, etc.) of the paddle width adjustment element to adjust the width of a paddle of the device.

In some implementations, the paddle actuation knob is rotated to cause axial movement of the actuation element to move the device from the open configuration to a closed configuration.

As part of the method, the device can be decoupled from the actuation element, the paddle width adjustment element, and the sheath.

In some implementations, the paddle actuation knob is rotated to move the device from a fully elongated configuration to the open configuration and the paddle actuation knob is further rotated in the same direction to move the device from the open configuration to the closed configuration.

In some implementations, the paddle width adjustment element is coupled to the at least one of the pair of paddles through an inner end. Axial movement of the paddle width adjustment element causes axial movement of the inner end with respect to an actuation portion of the device. Axial movement of the inner end causes the at least one of the pair of paddles to move relative to the actuation portion of the device effective to move the width of at least one of the pair of paddles from the first width to the second width.

In some implementations, rotating the paddle actuation knob is effective to axially move the paddle actuation knob and the paddle width control knob relative to the housing of the handle.

In some implementations, rotating the paddle width control knob causes axial movement of the paddle width control knob and the paddle width adjustment element relative to a frame coupled to the paddle actuation knob.

In some implementations, the paddle width adjustment element is rotationally fixed during rotation of the paddle width control knob and the actuation element is rotationally fixed during rotation of the paddle actuation knob.

In some implementations, the device is decoupled by pulling the adjustment through the actuation element and pulling the actuation element through the sheath.

Any of the above method(s) can be performed on a living subject (e.g., human or other animal) or on a simulation (e.g., a cadaver, cadaver heart, anthropomorphic ghost, simulator, etc.). With a simulation, the body parts can optionally be referred to as “simulated” (e.g., simulated heart, simulated tissue, etc.) and can comprise, for example, computerized and/or physical representations.

In some implementations, a handle assembly for controlling a transvascular device (e.g., a treatment device, a repair device, an implant, an implantable device, a valve repair device, etc.) includes a handle housing, a sheath extending distally from the handle housing, an actuation element extending through the sheath and configured to be coupled to the device, a paddle actuation control operatively coupled to the actuation element. In some implementations, the handle assembly also comprises a paddle width adjustment element extending through the actuation element and configured to be coupled to at least one of a pair of paddles of the device, and a paddle width control operatively coupled to the paddle width adjustment element. In some implementations, the handle assembly also includes a release control operatively coupled to the paddle width adjustment element.

In some implementations, the handle assembly is configured such that actuation of the paddle actuation control causes axial movement of the actuation element with respect to the handle housing and the sheath. Axial movement of the actuation element causes the device to move between open and closed positions.

In some implementations, the handle assembly is configured such that actuation of the paddle width control causes axial movement of the paddle width adjustment element with respect to the handle housing and the sheath. Axial movement of the paddle width adjustment element causes a width of the at least one of the pair of paddles of the device to be moved from a first width to a second width.

In some implementations, the handle assembly is configured such that actuation of the release control caused axial movement of the paddle width adjustment element and the actuation element to decouple the device from the sheath. Actuation of the paddle actuation control causes axial movement of the release control.

In some implementations, a delivery system (e.g., a delivery system for an implantable device, a delivery system for a repair device, a delivery system for a treatment device, etc.) includes a steerable catheter assembly having a handle and a sheath extending from the handle in an axial direction and a device catheter assembly (e.g., an implant catheter assembly, a control catheter assembly, a device control catheter assembly, etc.) having a handle and a sheath extendable coaxially through the sheath of the steerable catheter assembly. The handle of the device catheter assembly includes a handle housing, a sheath extending distally from the handle housing, an actuation element extending through the sheath and configured to be coupled to a device (e.g., a treatment device, a repair device, an implant, an implantable device, a valve repair device, etc.), and a paddle actuation control operatively coupled to the actuation element. In some implementations, the delivery system also includes a paddle width adjustment element extending through the actuation element and configured to be coupled to at least one of a pair of paddles of the device, and a paddle width control operatively coupled to the paddle width adjustment element. In some implementations, the delivery system also includes a release control operatively coupled to the paddle width adjustment element.

In some implementations, the delivery system is configured such that actuation of the paddle actuation control causes movement (e.g., one or more of axial movement, rotational movement, translational movement, etc.) of the actuation element with respect to the handle housing and the sheath. The movement of the actuation element causes the device to move between open and closed positions.

In some implementations, the delivery system is configured such that actuation of the paddle width control causes movement of the paddle width adjustment element with respect to the handle housing and the sheath. The movement of the paddle width adjustment element causes a width of the at least one of the pair of paddles of the device to be moved from a first width to a second width.

In some implementations, the delivery system is configured such that actuation of the release control can cause movement of the paddle width adjustment element and the actuation element to decouple the device from the sheath.

In some implementations, the delivery system is configured such that actuation of the paddle actuation control causes axial movement of the release control.

In some implementations, a method of delivering and/or using a device (e.g., a treatment device, a repair device, an implant, an implantable device, a valve repair device, etc.) includes obtaining the device coupled to an actuation element and a paddle width adjustment element extending from a distal end of a sheath of a catheter assembly (e.g., a device catheter assembly, an implant catheter assembly, a steerable catheter assembly, a control catheter assembly, a device control catheter assembly, etc.), advancing the sheath of the catheter assembly to position the device at a delivery site in an open configuration, actuating the paddle actuation control on the handle, thereby causing movement of the actuation element to move the device from the open configuration to a closed configuration, actuating a paddle width control on the handle, thereby causing movement of the paddle width adjustment element to move a width of at least one of a pair of paddles from a first width to a second width, and actuating a release control on the handle, thereby causing movement of the paddle width adjustment element and the actuation element to decouple the device from the sheath. In some implementations, actuating of the paddle actuation control causes axial movement of the release control and the paddle width adjustment element.

Any of the above method(s) can be performed on a living subject (e.g., human or other animal) or on a simulation (e.g., a cadaver, cadaver heart, anthropomorphic ghost, simulator, etc.). With a simulation, the body parts can optionally be referred to as “simulated” (e.g., simulated heart, simulated tissue, etc.) and can comprise, for example, computerized and/or physical representations.

In some implementations, a delivery system for a device (e.g., for an implantable device, for a repair device, for a treatment device, etc.) has a plurality of clasps for securing native leaflets of a heart valve includes a catheter assembly (e.g., a device catheter assembly, an implant catheter assembly, a steerable catheter assembly, a control catheter assembly, a device control catheter assembly, etc.) having a handle and a sheath extending from the handle in an axial direction and having a distal end portion comprising a capture mechanism for attaching the sheath to a device (e.g., a treatment device, a repair device, an implant, an implantable device, a valve repair device, etc.). The delivery system includes a first clasp actuation line configured to move a first clasp of the plurality of clasps between a closed position and an open position, the first clasp actuation line extending from the handle, through the sheath, and through a first aperture in the first clasp. A first distal end of the first clasp actuation line is attached to the capture mechanism when the capture mechanism is in the coupled position.

In some implementations, a method of using a device, e.g., valve repair device etc., on a heart valve of patient includes closing the first clasp of the device to grasp a first leaflet of the heart valve by releasing tension in the first clasp actuation line, closing the second clasp of the device to grasp a second leaflet of the heart valve by releasing tension in the second clasp actuation line, and releasing the device from the capture mechanism and withdrawing the capture mechanism from the device. Releasing the device from the capture mechanism and withdrawing the capture mechanism from the device releases the first clasp actuation line from the first clasp and the and the capture mechanism and releases the second clasp actuation line from both the second clasp and the capture mechanism.

Any of the above method(s) can be performed on a living subject (e.g., human or other animal) or on a simulation (e.g., a cadaver, cadaver heart, anthropomorphic ghost, simulator, etc.). With a simulation, the body parts can optionally be referred to as “simulated” (e.g., simulated heart, simulated tissue, etc.) and can comprise, for example, computerized and/or physical representations.

In some implementations, a handle assembly for controlling a transvascular device having a plurality of clasps for securing native leaflets of a heart valve includes a handle housing, a sheath extending distally from the handle housing, a first clasp actuation line extending through the sheath, the first clasp actuation line operatively coupled to a first clasp of the plurality of clasps on the device, and a first clasp control member operatively connected to the first clasp actuation line, the first clasp control member movable relative to the housing between a first position associated with the first clasp being in an open position and a second position associated with the clasp being in a closed position. The first clasp control member is biased to the second position.

In some implementations, a method of using a device or implant, such as a valve repair device, having a plurality of clasps for securing native leaflets of a heart valve includes delivering the device to the heart valve via a catheter assembly (e.g., a device catheter assembly, an implant catheter assembly, a steerable catheter assembly, a control catheter assembly, a device control catheter assembly, etc.) having a first clasp actuation line that holds a first clasp of the device in an open position and a second clasp actuation line that holds a second clasp of the device in an open position, closing the first clasp of the device to grasp a first leaflet of the heart valve by releasing tension in the first clasp actuation line, and closing the second clasp of the device to grasp a second leaflet of the heart valve by releasing tension in the second clasp actuation line. Releasing tension in the first clasp actuation line comprising biasing a first clasp control member from a first position to a second position releasing tension in the second clasp actuation line comprising biasing a second clasp control member from a third position to a fourth position.

Any of the above method(s) can be performed on a living subject (e.g., human or other animal) or on a simulation (e.g., a cadaver, cadaver heart, anthropomorphic ghost, simulator, etc.). With a simulation, the body parts can optionally be referred to as “simulated” (e.g., simulated heart, simulated tissue, etc.) and can comprise, for example, computerized and/or physical representations.

In some implementations, a clasp actuation line for actuating a clasp of device, such as a valve repair device, includes a braided body having a proximal end and a distal end opposite the proximal end, and a closed loop is formed in the distal end. In some implementations, the closed loop is formed by a bifurcated braided portion of the braided body. In some implementations, a distal terminal end of the braided body extends axially through a portion of the braided body at a location proximal the closed loop to form a tucked-in portion. In some implementations, a distal terminal end portion of the braided body extends laterally through a portion of the braided body at a location proximal the closed loop to form a threaded portion. In some implementations, a distal terminal end portion of the braided body extends axially through a portion of the braided body at a location proximal the closed loop to form a tucked-in portion proximal a threaded portion.

In some implementations, a method of forming a clasp actuation line for actuating a clasp of a device includes braiding an elongated body and forming a closed loop at a distal end of the elongated body. In some implementations, braiding the elongated body includes braiding a bifurcated portion to form the closed loop. In some implementations, braiding the elongated body further comprising braiding a plurality of a bifurcated portions separated by unitary portions and cutting the elongated body into sections that have a single bifurcated portion adjacent a distal end of the section. In some implementations, forming the closed loop comprises extending a distal terminal end of the braided body axially through a portion of the braided body to form a tucked-in portion proximal the closed loop. In some implementations, forming the closed loop comprises extending a distal terminal end portion of the braided body laterally through a portion of the braided body to form a threaded portion at a location proximal the closed loop.

Any of the above method(s) can be performed on a living subject (e.g., human or other animal) or on a simulation (e.g., a cadaver, cadaver heart, anthropomorphic ghost, simulator, etc.). With a simulation, the body parts can optionally be referred to as “simulated” (e.g., simulated heart, simulated tissue, etc.) and can comprise, for example, computerized and/or physical representations.

In some implementations, there is a provided a handle assembly (which can be the same as or similar to other handle assemblies described herein) for controlling a device (which can be the same as or similar to other devices described herein), the handle assembly comprising a handle housing, a sheath extending distally from the handle housing, and an actuation element (which can be the same as or similar to other actuation elements described herein) extending through at least a portion of the sheath, the actuation element configured to be coupled to the device. In some implementations, the handle assembly also includes a width adjustment element (which can be the same as or similar to other width adjustment elements described herein) extending through at least a portion of the sheath, the width adjustment element configured to be coupled to at least one of a pair of anchors (which can be the same as or similar to other anchors, paddles, clasps, etc. described herein) of the device. In some implementations, the handle assembly also includes an actuation control (which can be the same as or similar to other actuation controls described herein) coupled to the actuation element, wherein actuation of the actuation control causes movement of the actuation element with respect to the handle housing and/or the sheath, wherein the movement of the actuation element can move the device between an open configuration and a closed configuration.

In some implementations, the handle assembly further comprises a width control (which can be the same as or similar to other width controls, such as paddle width controls, described herein) coupled to the width adjustment element, wherein actuation of the width control causes movement of the width adjustment element relative to the handle housing and/or the sheath, wherein the movement of the width adjustment element can transition at least one of the pair of anchors of the device between a first width and a second width.

In some implementations, the handle assembly further comprises one or more clasp actuation lines (which can be the same as or similar to other actuation lines described herein) extending through the sheath, the one or more clasp actuation lines configured to be coupled to the device.

In some implementations, the handle assembly further comprises one or more clasp control members (which can be the same as or similar to other clasp control members described herein), wherein the one or more clasp control members are movable relative to the handle housing, wherein movement of the one or more clasp control members causes one or more clasps of the device to be moved between an open configuration and a closed configuration.

In some implementations, the one or more clasp actuation lines are coupled to a suture lock extending from a proximal end of the handle housing.

In some implementations, the one or more clasp actuation lines include a braided body having a proximal end and a distal end opposite the proximal end, and a closed loop is formed in the distal end. In some implementations, the closed loop is formed by a bifurcated braided portion of the braided body. In some implementations, a distal terminal end of the braided body extends axially through a portion of the braided body at a location proximal the closed loop to form a tucked-in portion. In some implementations, a distal terminal end portion of the braided body extends laterally through a portion of the braided body at a location proximal the closed loop to form a threaded portion. In some implementations, a distal terminal end portion of the braided body extends axially through a portion of the braided body at a location proximal the closed loop to form a tucked-in portion proximal a threaded portion.

In some implementations, each suture lock is angled with respect to a central axis extending through the handle assembly.

In some implementations, the width control is coupled to a planetary gearbox and actuation of the width control is effective to cause rotation of the planetary gearbox. In some implementations, the planetary gearbox comprises at least a ring gear, a pair of planet gears, and an elongated central gear. In some implementations, the elongated central gear is coupled to the width adjustment element through a rotationally fixed follower such that rotation of the elongated central gear causes axial movement of the rotationally fixed follower, which in turn causes axial movement of the width adjustment element with respect to the housing. In some implementations, external teeth of the elongated central gear engage with teeth of the pair of planet gears.

In some implementations, a handle assembly (which can be the same as or similar to other handle assemblies described herein) for controlling a transvascular device (e.g., a treatment device, a repair device, an implant, an implantable device, a valve repair device, etc.) includes a handle housing, a sheath, a first clasp actuation element, a first clasp control member, and a first biasing element. The sheath extends distally from the handle housing. The first clasp actuation element extends through the sheath and is operatively coupled to a first clasp of a plurality of clasps on the device.

In some implementations, the first clasp control member is operatively connected to the first clasp actuation element and is movable relative to the housing between a first position associated with the first clasp being in an open position and a second position associated with the first clasp being in a closed position.

In some implementations, the first biasing element is configured to apply a force to pull the first clasp toward the open position.

The handle assembly can be part of a catheter assembly (e.g., a device catheter assembly, an implant catheter assembly, a steerable catheter assembly, a control catheter assembly, a device control catheter assembly, etc.) of a delivery system for the device. The delivery system can include a sheath extending from the handle assembly in an axial direction. The sheath can include a distal end portion comprising a capture mechanism for releasably attaching the sheath to the device.

In some implementations, the first biasing element can apply the force onto the first clasp actuation element. The first biasing element can directly contact the first clasp actuation element when applying the force. The force can be directed radially outward from a centerline of the handle housing.

In some implementations, the force is not applied to pull the first clasp toward the open position when the first clasp control member is in the second position.

In some implementations, the first biasing element can have an elongated body having a proximal end fixed relative to the handle housing and a free distal end. The elongated body can have a semi-elliptical shape. The elongated body can have an opening extending laterally through the elongated body and the first clasp actuation element can extend through the opening. In some implementations, the opening can be positioned closer to the distal end than the proximal end.

In some implementations, the first clasp actuation element is a suture line.

In some implementations, the first biasing element has a wide position in which the force is applied to pull the first clasp toward the open position and a narrow position in which the force is not applied to pull the first clasp toward the open position. The first biasing element can be biased to the wide position. In some implementations, the first clasp control member holds the first biasing element in the narrow position when the first clasp control member is in the second position. In some implementations, the first clasp control member releases the first biasing element to the wide position when the first clasp control member is in the first position.

In some implementations, the first biasing element is positioned in-line with the first clasp actuation element.

In some implementations, the first biasing element comprises an elastic portion of the first clasp actuation element. The elastic portion can extend along an entire length of the first clasp actuation element or can extend along a partial length of the first clasp actuation element. The elastic portion of the first clasp actuation element can be positioned inside the handle housing.

In some implementations, the first biasing element does not apply the force to pull the first clasp toward the open position when the first clasp control member is in the second position.

In some implementations, the first clasp control member is movable axially relative to the housing between the first position and the second position.

In some implementations, the handle assembly includes a second clasp actuation element, a second clasp control member, and a second biasing element. The second clasp actuation element can extend through the sheath and be operatively coupled to a second clasp of the plurality of clasps on the device.

In some implementations, the second clasp control member can be operatively connected to the second clasp actuation element and be movable relative to the housing between a third position associated with the second clasp being in an open position and a fourth position associated with the second clasp being in a closed position.

The second biasing element can be configured to apply a second force to pull the second clasp toward the open position.

In some implementations, the second biasing element is configured to apply the second force independent of the first biasing element.

In some implementations, a method of using a device, e.g., a valve repair device etc., having one or more clasps for securing native leaflets of a heart valve includes delivering the device to the heart valve via a catheter assembly (e.g., a device catheter assembly, an implant catheter assembly, a steerable catheter assembly, a control catheter assembly, a device control catheter assembly, etc.), holding a first clasp of the device in an open position with a first clasp actuation element, and closing the first clasp of the device to grasp a first leaflet of the heart valve.

In some implementations, a first clasp control member is moved to a first position to hold the first clasp of the device in an open position and moved to a second position to close the first clasp.

Holding the first clasp of the device in an open position can include applying a force to the first clasp actuation element after the first clasp control member is in the first position.

In some implementations, applying the force to the first clasp actuation element includes applying a force onto the first clasp actuation element. The force can be a radially outward force. In some implementations, the first clasp actuation element is a suture line.

In some implementations, applying the force to the first clasp actuation element after the first clasp control member is in the first position includes moving a biasing element to a wide position. Moving the biasing element to the wide position can include moving the first clasp control member to the first position.

In some implementations, moving the first clasp control member to the second position includes moving the biasing element to a narrow position. Moving the biasing element to the narrow position can include engaging the biasing element with the first clasp control member. In some implementations, the biasing element is held in the narrow position with the first clasp control member.

In some implementations, moving the first clasp control member to the second position includes moving the first clasp control member axially.

In some implementations, applying force to the first clasp actuation element includes stretching an elastic portion of the first clasp actuation element.

In some implementations, the method includes holding a second clasp of the device in an open position with a second clasp actuation element and closing the second clasp of the device to grasp a second leaflet of the heart valve.

In some implementations, a second clasp control member is moved to a third position to hold the second clasp of the device in the open position and is moved to a fourth position to close the first clasp. Holding the second clasp of the device in the open position can include applying a second force to the second clasp actuation element after the second clasp control member is in the third position.

In some implementations, the first force is applied independent of the second force.

In some implementations, a clasp actuation line for actuating a clasp of a device via a clasp control member includes a braided body having a first end configured to operatively couple to the clasp control member and a second end opposite the first end. The braided body can have a first portion having a first elasticity and a second portion having a second elasticity greater than the first elasticity.

In some implementations, the first portion of the braided body has a first number of picks per inch and the second portion of the braided body has a second number of picks per inch that is greater than the first number of picks per inch. Both of the first portion and the second portion can be formed from an ultra-high-molecular-weight polyethylene material.

In some implementations, the second portion can extend the majority of an entire length of the clasp actuation line. In some implementations, the second portion is adjacent the first end and extends less than half a total length of the clasp actuation line.

Any of the above method(s) can be performed on a living subject (e.g., human or other animal) or on a simulation (e.g., a cadaver, cadaver heart, anthropomorphic ghost, simulator, etc.). With a simulation, the body parts can optionally be referred to as “simulated” (e.g., simulated heart, simulated tissue, etc.) and can comprise, for example, computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of certain examples and implementations will be made by reference to various aspects of the appended drawings. These drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase.

FIG. 2 illustrates a cutaway view of the human heart in a systolic phase.

FIG. 3 illustrates a cutaway view of the human heart in a systolic phase showing valve regurgitation.

FIG. 4 is the cutaway view of FIG. 3 annotated to illustrate a natural shape of mitral valve leaflets in the systolic phase.

FIG. 5 illustrates a healthy mitral valve with the leaflets closed as viewed from an atrial side of the mitral valve.

FIG. 6 illustrates a dysfunctional mitral valve with a visible gap between the leaflets as viewed from an atrial side of the mitral valve.

FIG. 7 illustrates a tricuspid valve viewed from an atrial side of the tricuspid valve.

FIGS. 8-14 show an example of a device or implant, in various stages of deployment.

FIG. 15 shows an example of a device or implant that is similar to the device illustrated by FIGS. 8-14, but where the paddles are independently controllable.

FIGS. 16-21 show the example device or implant of FIGS. 8-14 being delivered and implanted within a native valve.

FIG. 22 shows a perspective view of an example device or implant in a closed position.

FIG. 23 shows a front view of the device or implant of FIG. 22.

FIG. 24 shows a side view of the device or implant of FIG. 22.

FIG. 25 shows a front view of the device or implant of FIG. 22 with a cover covering the paddles and a coaptation element or spacer.

FIG. 26 shows a top perspective view of the device or implant of FIG. 22 in an open position.

FIG. 27 shows a bottom perspective view of the device or implant of FIG. 22 in an open position.

FIG. 28 shows a clasp for use in a device or implant.

FIG. 29 shows a portion of native valve tissue grasped by a clasp.

FIG. 30 shows a side view of an example device or implant in a partially-open position with clasps in a closed position.

FIG. 31 shows a side view of an example device or implant in a partially-open position with clasps in an open position.

FIG. 32 shows a side view of an example device or implant in a half-open position with clasps in a closed position.

FIG. 33 shows a side view of an example device or implant in a half-open position with clasps in an open position.

FIG. 34 shows a side view of an example device or implant in a three-quarters-open position with clasps in a closed position.

FIG. 35 shows a side view of an example device or implant in a three-quarters-open position with clasps in an open position.

FIG. 36 shows a side view of an example device in a fully open or full bailout position with clasps in a closed position.

FIG. 37 shows a side view of an example device in a fully open or full bailout position with clasps in an open position.

FIGS. 38-49 show the example device or implant of FIGS. 30-38, including a cover being delivered and implanted within a native valve.

FIG. 50 is a schematic view illustrating a path of native valve leaflets along each side of a coaptation element or spacer of an example device or implant.

FIG. 51 is a top schematic view illustrating a path of native valve leaflets around a coaptation element or spacer of an example device or implant.

FIG. 52 illustrates a coaptation element or spacer in a gap of a native valve as viewed from an atrial side of the native valve.

FIG. 53 illustrates a device or implant attached to native valve leaflets with the coaptation element or spacer in the gap of the native valve as viewed from a ventricular side of the native valve.

FIG. 54 is a perspective view of a device or implant attached to native valve leaflets with the coaptation element or spacer in the gap of the native valve shown from a ventricular side of the native valve.

FIG. 55 shows a perspective view of an example device or implant in a closed position.

FIG. 56 shows a perspective view of an example clasp of an example device or implant in a closed position.

FIG. 57 illustrates a device with paddles in an open position.

FIG. 58 illustrates the device of FIG. 57, in which the paddles are in the open position and gripping members are moved to create a wider gap between the gripping members and paddles.

FIG. 59 illustrates the device of FIG. 57, in which the device is in the position shown in FIG. 57 with valve tissue placed between the gripping members and the paddles.

FIG. 60 illustrates the device of FIG. 57, in which the gripping members are moved to lessen the gap between the gripping members and the paddles.

FIGS. 61A-61B illustrate the movement of the paddles of the device of FIG. 57 from the open position to a closed position.

FIG. 62 illustrates the device of FIG. 57 in a closed position, in which the gripping members are engaging valve tissue.

FIG. 63 illustrates the device of FIG. 57 after being disconnected from a delivery device and attached to valve tissue, in which the device is in a closed and locked condition.

FIG. 64 illustrates a distal end of an example delivery assembly including a delivery apparatus and a device.

FIG. 65 illustrates a proximal end of the example delivery assembly of FIG. 64.

FIG. 66 illustrates an example catheter assembly for use in a delivery assembly coupled to a device.

FIG. 67 illustrates a perspective view of an example of a device having paddles of adjustable widths, and shows some aesthetic features.

FIG. 68 is a cross-section of the device of FIG. 67 in which the device is bisected, and shows some aesthetic features.

FIG. 69 is another cross-section of the device of FIG. 67 in which the device is bisected along a plane perpendicular to the plane shown in FIG. 68, and shows some aesthetic features.

FIG. 70 is a schematic illustration of an example catheter assembly coupled to a device, in which an actuation element, such as a tube is coupled to a paddle actuation control and to a driver head of the device, and shows some aesthetic features.

FIG. 71 is an illustration of the assembly of FIG. 70 with the device rotated 90 degrees to show the paddle width adjustment element coupled to an inner end of the device and coupled to a paddle width control, and shows some aesthetic features.

FIG. 72 is an example catheter assembly that can be used in conjunction with the handles of various implementations described herein, and shows some aesthetic features.

FIG. 73 is a perspective view of an example handle of a catheter assembly, and shows some aesthetic features.

FIG. 74 is an enlarged portion of FIG. 73, and shows some aesthetic features.

FIG. 75 is a cross-section of the portion of the handle of FIG. 73, and shows some aesthetic features.

FIG. 76 is a schematic illustration of an example catheter assembly coupled to a device, in which each of the clasp actuation lines is coupled to a clasp control member positioned on the handle and the actuation element is coupled to a knob positioned on the handle, and shows some aesthetic features.

FIG. 77 is a perspective view of an example handle of a catheter assembly, and shows some aesthetic features.

FIG. 78 is a cross-section of the handle of FIG. 77 perpendicular to the plane defined by line A-A, in which one of the clasp control tubes is bisected, and shows some aesthetic features.

FIG. 79 is another cross-section of the handle of FIG. 77 perpendicular to the plane defined by line B-B, in which the release knob is bisected, and shows some aesthetic features.

FIG. 80 is an enlarged portion of FIG. 79, and shows some aesthetic features.

FIG. 81 is another cross-section of the handle of FIG. 77 perpendicular to the plane defined by line C-C, in which the release knob and the suture locks are bisected, and shows some aesthetic features.

FIG. 82 is another perspective view of the handle of FIG. 77 in which the housing includes a pair of detents for fixing the clasp control members into position, and shows some aesthetic features.

FIG. 83 is a perspective view of an example proximal end of a handle in which the release knob is in a distal position, and shows some aesthetic features.

FIG. 84 is a perspective view of an example proximal end of a handle in which the release knob is in a proximal position, and shows some aesthetic features.

FIG. 85 is a perspective view of a partial cut away of a handle including an example release knob having a ratcheting mechanism, and shows some aesthetic features.

FIG. 86 is a perspective view of the release knob of FIG. 85 having the ratcheting mechanism, and shows some aesthetic features.

FIG. 87 is a cross-sectional view of the release knob of FIGS. 85-86 perpendicular to the plane defined by line D-D in which the pawls are sectioned, and shows some aesthetic features.

FIG. 88 is a cross-sectional view of an example suture lock in which a clasp actuation line is fixed to a post at one end and extends between a suture lock body and a suture lock body receptacle at a second end after having been coupled to a device, and shows some aesthetic features.

FIG. 89 is a perspective view of an example handle including a paddle width control knob and a cap over the suture locks, and shows some aesthetic features.

FIG. 90 is an enlarged view of a portion of the handle in FIG. 89 without the cap, and shows some aesthetic features.

FIG. 91 is a perspective view of an elongated central gear of a planetary gearbox coupled to a follower for use in one or more implementations shown and described herein, and shows some aesthetic features.

FIG. 92 is another view of the elongated central gear and the follower for use in one or more implementations shown and described herein, and shows some aesthetic features.

FIG. 93 is a perspective view of the follower extending from the proximal end of the handle, and shows some aesthetic features.

FIG. 94 is a perspective view of the frame rotationally fixing the follower with respect to the handle, and shows some aesthetic features.

FIG. 95 is a perspective view of the elongated central gear extending from the proximal end of the handle, and shows some aesthetic features.

FIG. 96 is a perspective view of the carrier and planet gear of the planetary gear box for use in one or more implementations shown and described herein, and shows some aesthetic features.

FIG. 97 is a perspective view of the planetary gearbox positioned within the handle, and shows some aesthetic features.

FIG. 98 is an end view showing the planetary gearbox within the handle, and shows some aesthetic features.

FIG. 99 is a perspective view of the handle including the housing without the cap, and shows some aesthetic features.

FIG. 100 is a perspective view of an example handle of a catheter assembly, and shows some aesthetic features.

FIG. 101 is a partial cross-section view of the handle of FIG. 100, and shows some aesthetic features.

FIG. 102 is a partial cross-section view of the handle of FIG. 100, and shows some aesthetic features.

FIG. 103 is a partial cross-section view of the portion of the handle of FIG. 100 showing the clasp control members, and shows some aesthetic features.

FIG. 104 is a cross-section view of the portion of the handle of FIG. 103, and shows some aesthetic features.

FIG. 105 is a cross-section view of the portion of the handle of FIG. 103, and shows some aesthetic features.

FIG. 106 is a cross-section view of a portion of the handle of FIG. 100 showing the handle when an example device is an elongated or fully open condition, and shows some aesthetic features.

FIG. 107 is a perspective view of the example device in an elongated or fully open condition, and shows some aesthetic features.

FIG. 108 is a cross-section view of a portion of the handle of FIG. 100 showing the handle when the device is transitioning from the fully open condition to a fully closed position, and shows some aesthetic features.

FIG. 109 is a perspective view of the device in a partially open position, and shows some aesthetic features.

FIG. 110 is a perspective view of the device in a fully closed condition, and shows some aesthetic features.

FIG. 111 is a cross-section view of a portion of the handle of FIG. 100 showing the handle when the paddles of the device are in an expanded position, and shows some aesthetic features.

FIG. 112 is a perspective view of the device with the paddles in the expanded position, and shows some aesthetic features.

FIG. 113 is a cross-section view of a portion of the handle of FIG. 100 showing the handle when the paddles of the device are in a narrowed position, and shows some aesthetic features.

FIG. 114 is a perspective view of the device with the paddles in the narrowed position, and shows some aesthetic features.

FIG. 115 is a cross-section view of a portion of the handle of FIG. 100 showing an implant release knob, and shows some aesthetic features.

FIG. 116 is a cross-section view of the handle portion of FIG. 115 showing rotational movement of the release knob, and shows some aesthetic features.

FIG. 117 is a cross-section view of the handle portion of FIG. 115 showing axial movement of the release knob, and shows some aesthetic features.

FIG. 118 is a perspective view of a device in an elongated position illustrating an example of a capture mechanism and clasp control member routing, and shows some aesthetic features.

FIG. 119 is a side view of an example clasp actuation line, and shows some aesthetic features.

FIG. 120 is a schematic illustration of the device attached to the delivery system showing routing of the clasp actuation lines, and shows some aesthetic features.

FIG. 121 is the schematic illustration of FIG. 120 where the device is decoupled from the delivery system, and shows some aesthetic features.

FIGS. 122-124 are schematic illustrations of a device attached to the delivery system showing various stages of release of the device, and shows some aesthetic features.

FIG. 125-127 are perspective views of an example of a capture mechanism in various stages of release of a device, and shows some aesthetic features.

FIG. 128 is a side view of an example of a clasp actuation line.

FIG. 129 is a side view of an example of a clasp actuation line.

FIGS. 130-131 are side views of an example of a clasp actuation line.

FIGS. 132-133 are side views of an example of a clasp actuation line.

FIGS. 134-135 are side views of an example of a clasp actuation line.

FIGS. 136-137 are side views of an example of a clasp actuation line; and

FIG. 138 is a schematic illustration of a device attached to a delivery system.

FIG. 139 illustrates an example of a device or implant shown in a partially open, grasp-ready condition with the clasps in a fully open position.

FIG. 140 illustrates the device or implant of FIG. 139 with the clasps in a drooped position.

FIG. 141 illustrates an example of a device or implant shown in an elongated condition with the clasps in a fully open position.

FIG. 142 illustrates the device or implant of FIG. 141 with the clasps in a drooped position.

FIG. 143 is a schematic illustration of an example catheter assembly coupled to a device, in which each of the clasp actuation lines is coupled to a clasp control member that is biased to eliminate clasp droop.

FIG. 144 is a schematic illustration of an example catheter assembly coupled to a device, in which each of the clasp actuation line includes an elastic portion.

FIG. 145 illustrates an example clasp actuation portion of a catheter assembly device in which example clasp control members are in a first position.

FIG. 146 illustrates an example clasp actuation portion of a catheter assembly device in which example clasp control members are in a second position.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of treatment devices, repair devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the treatment techniques and methods herein can be performed on a living animal or on a simulation, such as on a cadaver, cadaver heart, simulator (e.g., with the body parts, heart, tissue, etc. being simulated), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

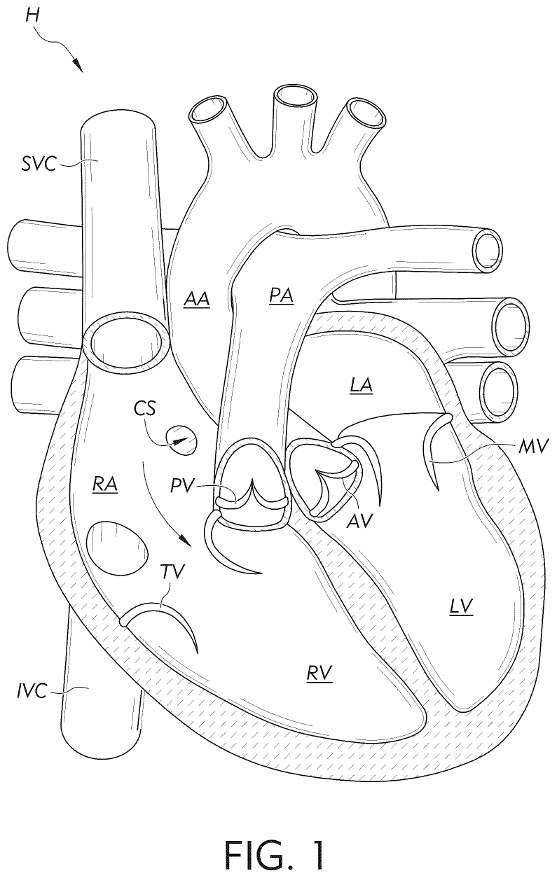

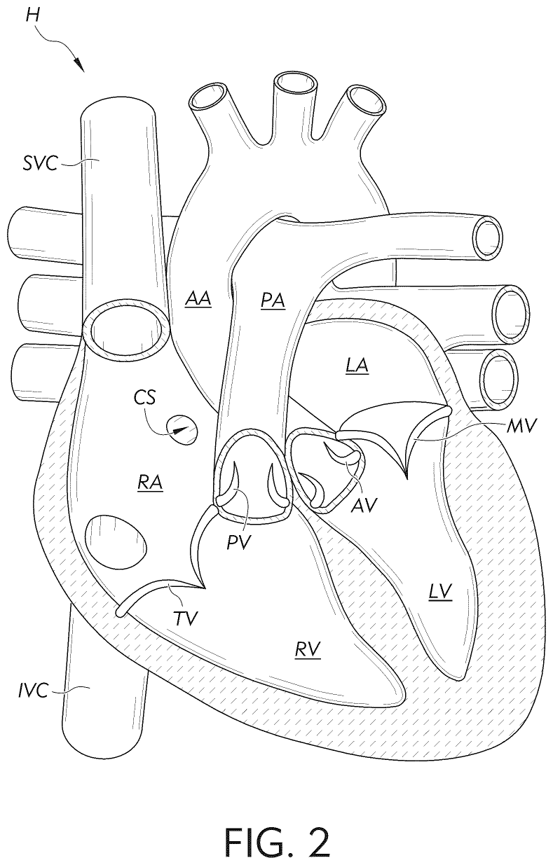

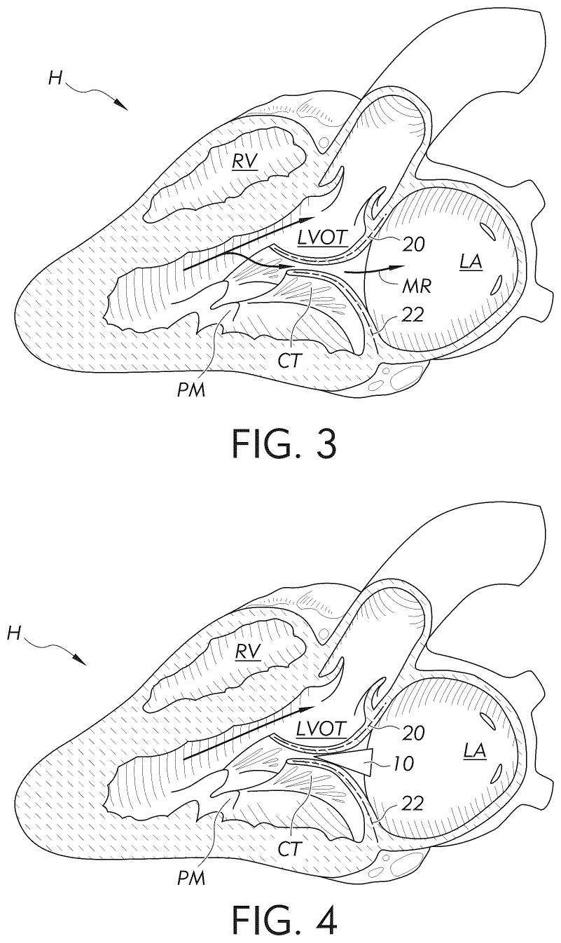

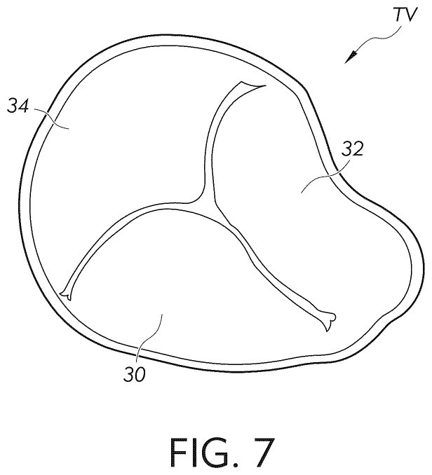

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit, or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

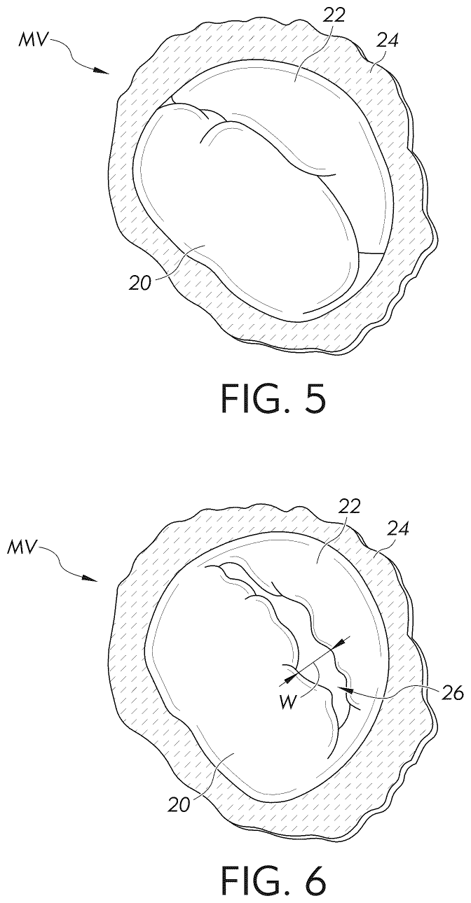

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which can thereby lead to valvular regurgitation.

In any of the above-mentioned situations, a device (e.g., an implant, a non-implantable device, a treatment device, a repair device, a valve repair device, etc.) is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a valve repair device, repair device, implantable device, or implant 10 is shown implanted between the leaflets 20, 22 such that regurgitation does not occur during systole (compare FIG. 3 with FIG. 4).

In some implementations, a coaptation element (e.g., spacer, coaption element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaptation element, e.g., spacer, coaption element, gap filler, etc. instead of only against one another).

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, the septal leaflet 32, and the posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices, treatment devices, or other devices and implants provided herein can be centrally located between the three leaflets 30, 32, 34.

An example device or implant can optionally have a coaptation element (e.g., spacer, coaption element, gap filler, etc.) and at least one anchor (e.g., one, two, three, or more). In some implementations, a device or implant can have any combination or sub-combination of the features disclosed herein without a coaptation element.

When included, the coaptation element (e.g., coaption element, spacer, etc.) can be configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral valve leaflets 20, 22 or tricuspid valve leaflets 30, 32, 34) that do not close completely.

The optional coaptation element (e.g., spacer, coaption element, gap filler, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element, such as a shaft, rod, tube, wire, etc., to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, a device/implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for various implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

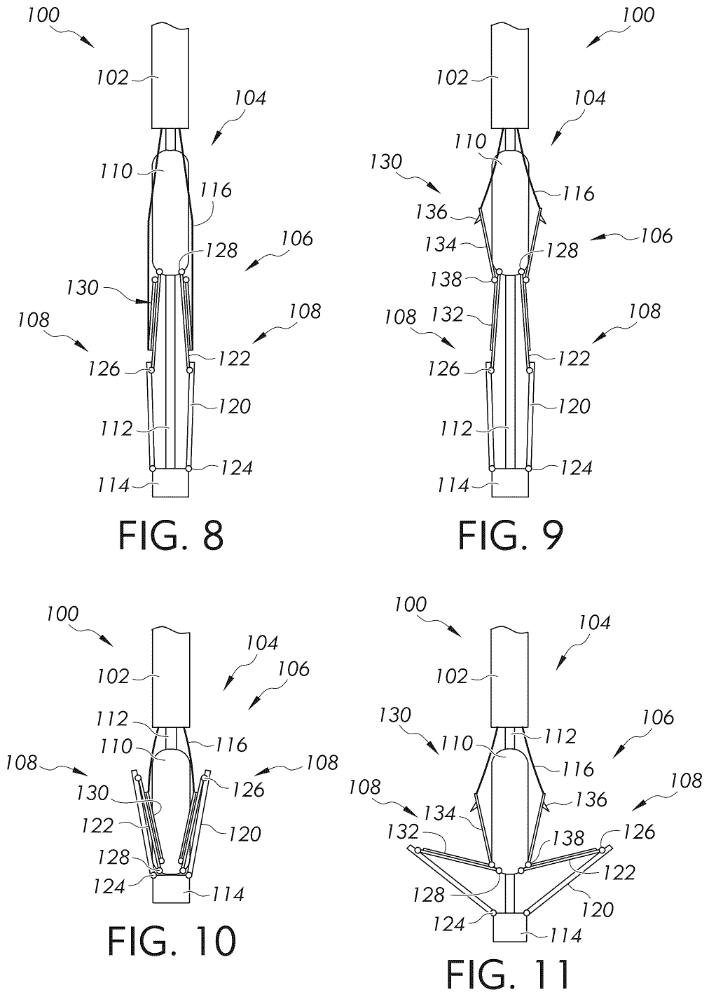

Referring now to FIGS. 8-15, a schematically illustrated device or implant 100 (e.g., an implantable prosthetic device, a prosthetic spacer device, a valve repair device, a treatment device, an implantable device, a repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for another device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application, or the applications cited above).

The device or implant 100 is deployed from a delivery system 102. The delivery system 102 can comprise one or more of a catheter, a sheath, a guide catheter/sheath, a delivery catheter/sheath, a steerable catheter, a device/implant catheter, a tube, a channel, a pathway, combinations of these, etc. The device or implant 100 includes a coaptation portion/coaptation region 104 and an anchor portion/anchor region 106.

In some implementations, the coaptation portion 104 of the device or implant 100 includes a coaptation element 110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other means for actuating and actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and the coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, device/implant catheter, and actuation element 112 (e.g., actuation wire, actuation shaft, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a movable arm 134, optional friction-enhancing elements, or other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate a coaptation element 110. The joint portion 138 provides a spring force between the fixed and movable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and movable arms 132, 134. The fixed arms 132 are attached to the inner paddles 122 and remain stationary or substantially stationary relative to the inner paddles 122 when the movable arms 134 are opened to open the clasps 130 and expose the optional barbs, friction-enhancing elements, or securing structures 136.

In some implementations, the clasps 130 are opened by applying tension to actuation lines 116 attached to the movable arms 134, thereby causing the movable arms 134 to articulate, flex, or pivot on the joint portions 138. The actuation lines 116 extend through the delivery system 102 (e.g., through a steerable catheter and/or a device/implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs, friction-enhancing elements, or securing structures 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, gap filler, etc.). The clasps 130 can be used to grasp and/or further secure the native leaflets by engaging the leaflets with optional barbs, friction-enhancing elements, or securing structures 136 and pinching the leaflets between the movable and fixed arms 134, 132. The optional barbs, friction-enhancing elements, or other securing structures 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system 102, so that the optional barbs, friction-enhancing elements, or other securing structures 136 (FIG. 9) do not catch or damage the delivery system 102. The actuation lines 116 can extend and attach to the movable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and movable portions 132, 134 of the clasps 130. Fully opening the paddles 120, 122 and the clasps 130 has been found to improve ease of detanglement or detachment from anatomy of the patient, such as the chordae tendineae CT, during implantation of the device 100.

Referring now to FIG. 10, the device 100 is shown in a shortened or fully closed condition. To move the device 100 from the elongated condition to the shortened condition, the actuation element 112 is retracted to pull the cap 114 towards the coaptation element 110 (e.g., towards a spacer). The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., wire, shaft, etc.) and cap (or other attachment portion) are used to control one paddle, and the other independent actuation element and cap (or other attachment portion) are used to control the other paddle.

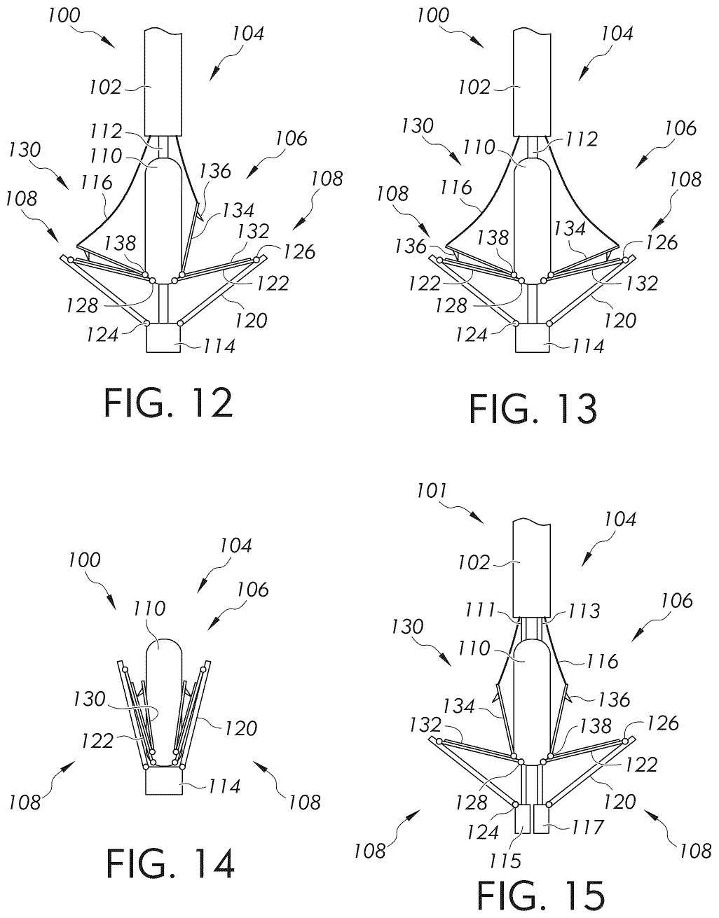

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and movable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device 100 illustrated by FIG. 11, except the device 101 of FIG. 15 includes an actuation element that is configured as two independent actuation elements (e.g., actuation shafts, actuation rods, actuation tubes, actuation wires, etc.) 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

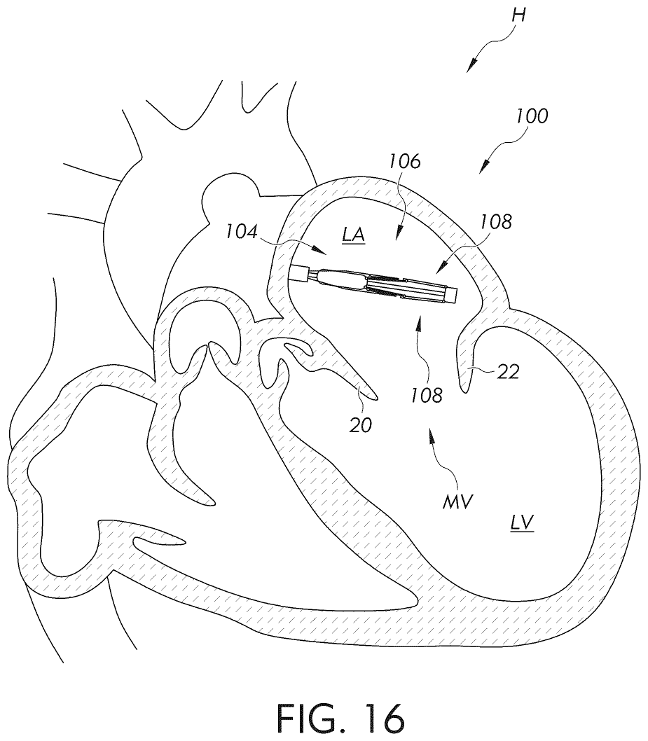

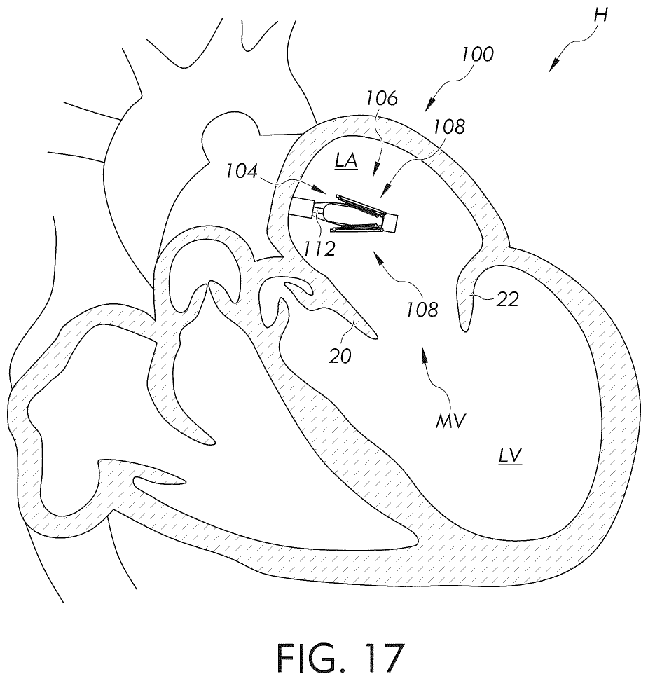

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

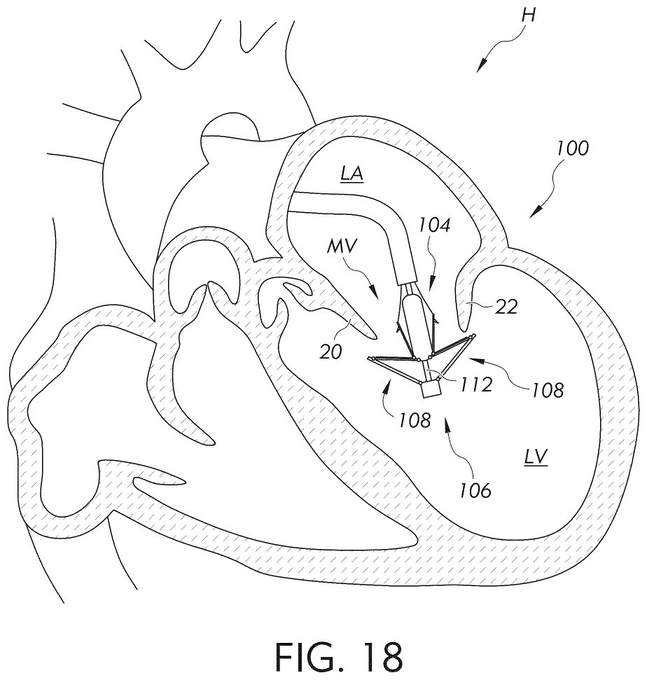

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant/device catheter connected to the implant/device can be advanced from inside the steerable catheter to position the implant as illustrated by FIG. 18.

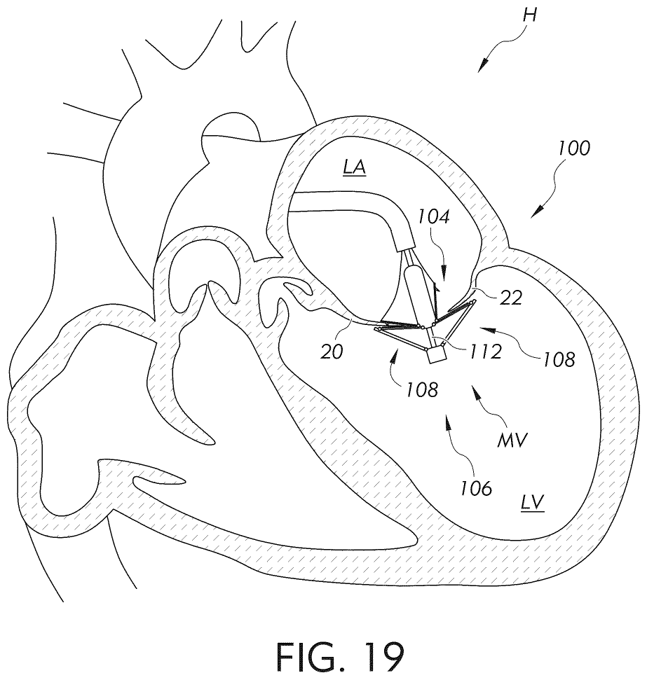

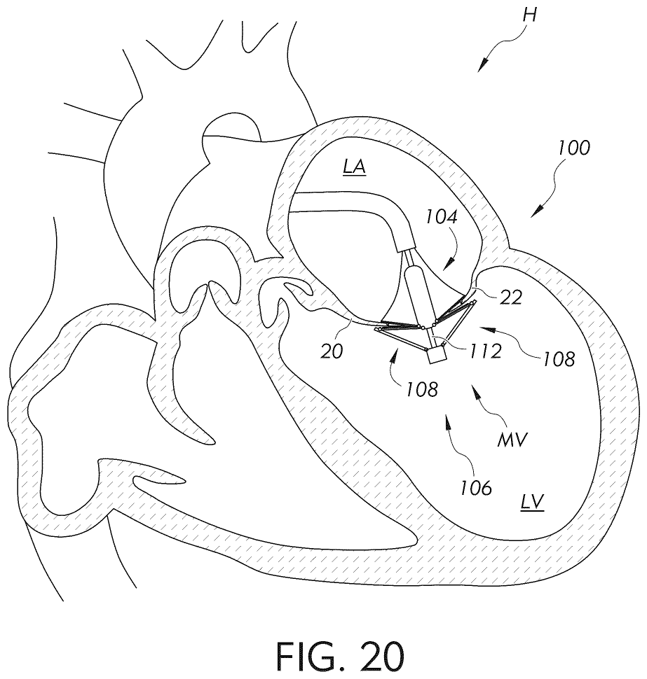

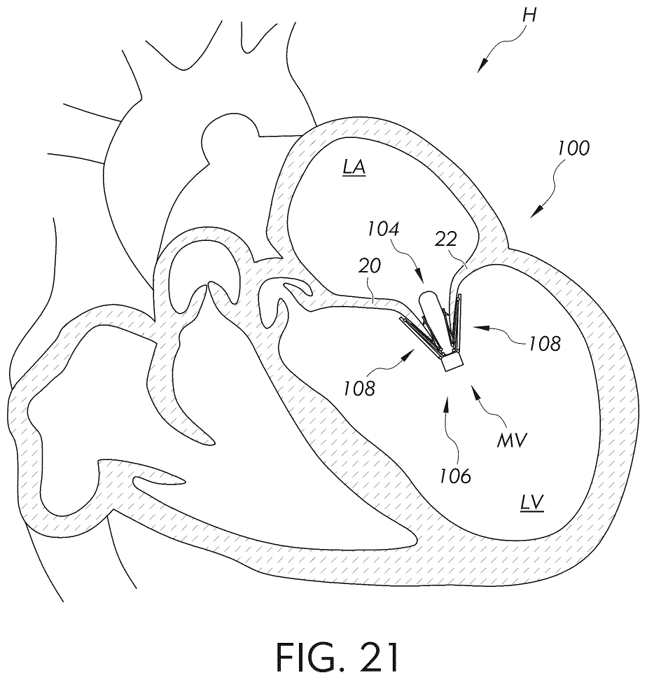

Referring now to FIG. 19, the implant/device catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant/device catheter, etc.), actuation element 112 and actuation lines 116 are then retracted and the device or implant 100 is fully closed and deployed in the native mitral valve MV.

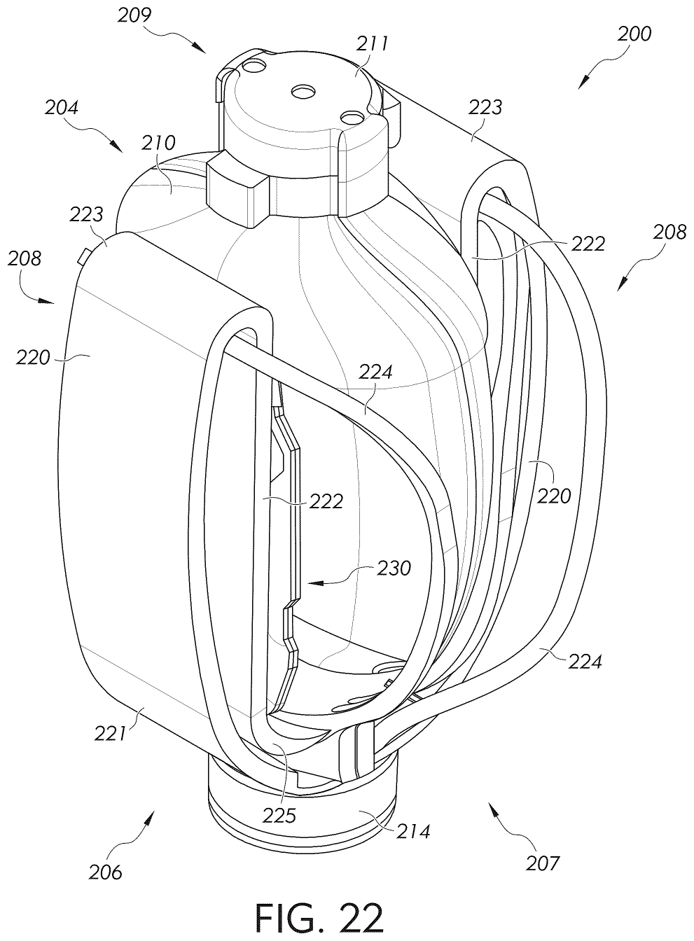

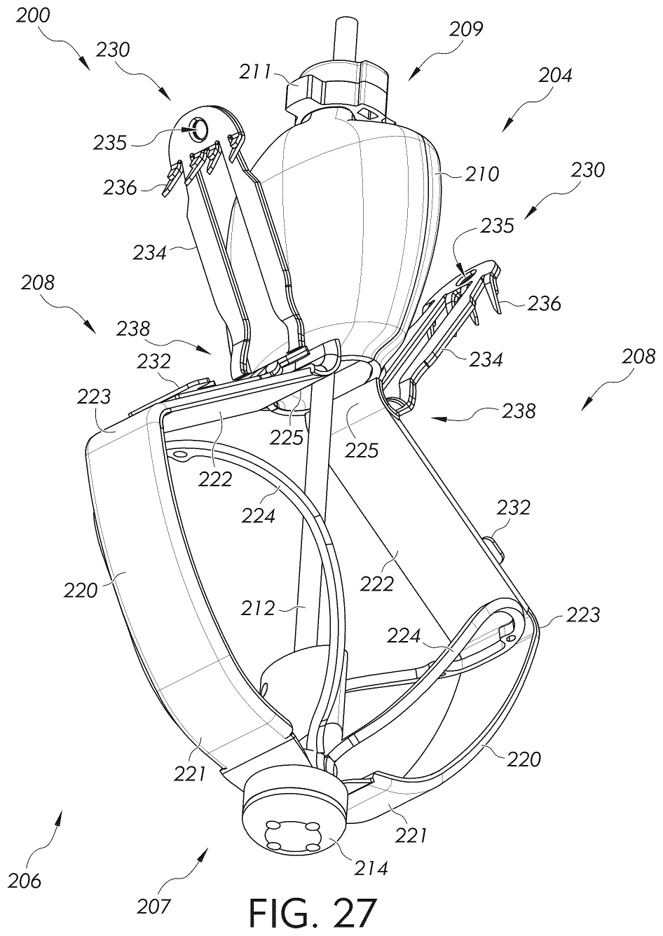

Any of the features disclosed by the present application can be used in a wide variety of different devices or valve repair devices (which can be implantable or non-implantable). FIGS. 22-27, 55, 57-63, and 67-71 illustrate examples of devices or valve repair devices that can be modified to include any of the features disclosed by the present application. Any combination or sub-combination of the features disclosed by the present application can be combined with, substituted for, and/or added to any combination or sub-combination of the features of the devices illustrated by FIGS. 22-27, 55, 57-63, and 67-71.

Referring now to FIGS. 22-27, an example of a device or implant 200 is shown. The device 200 is one of the many different configurations that the device 100 that is schematically illustrated in FIGS. 8-14 can take. The device 200 can include any other features for a device or implant discussed in the present application, and the device 200 can be positioned to engage valve tissue 20, 22 as part of any suitable valve repair system (e.g., any valve repair system disclosed in the present application). The device/implant 200 can be a treatment device, a prosthetic spacer device, valve repair device, or another type of device or implant that attaches to leaflets of a native valve.

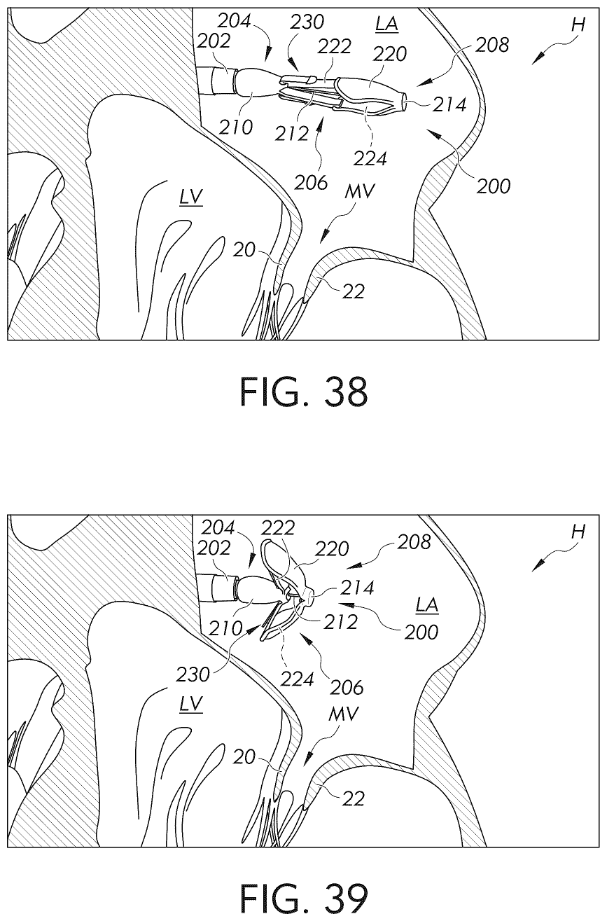

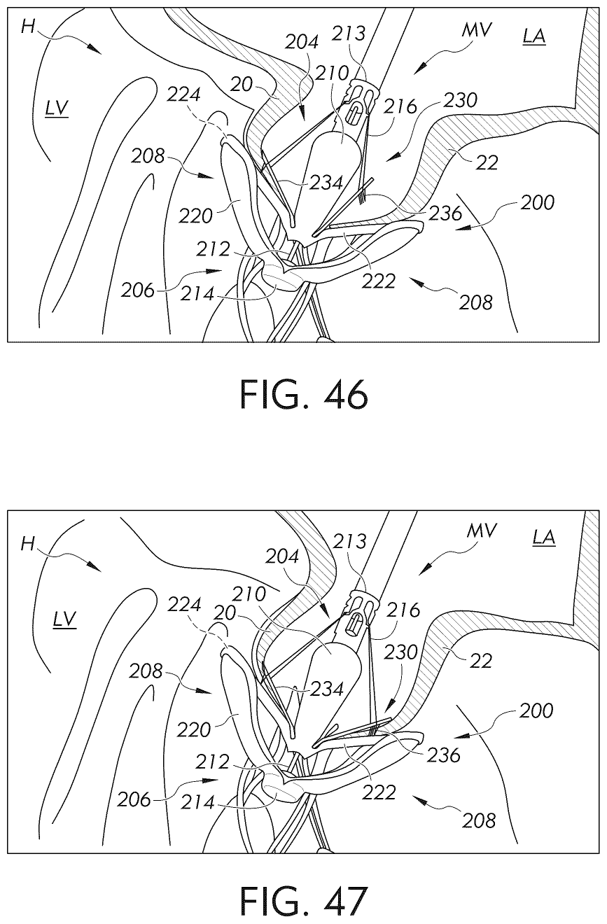

In some implementations, the device or implant 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism 213 (see e.g., FIGS. 43-49) of a delivery system 202 (see e.g., FIGS. 38-42 and 49). Delivery system 202 can be the same as or similar to delivery system 102 described elsewhere and can comprise one or more of a catheter, a sheath, a guide catheter/sheath, a delivery catheter/sheath, a steerable catheter, a device/implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element 212 (e.g., actuation shaft, actuation rod, actuation tube, actuation wire, actuation line, etc.) extends from the delivery system 202 to engage and enable actuation of the device or implant 200. In some implementations, the actuation element 212 extends through the capture mechanism 213, proximal collar 211, and coaptation element 210 to engage a cap 214 of the distal portion 207. The actuation element 212 can be configured to removably engage the cap 214 with a threaded connection, or the like, so that the actuation element 212 can be disengaged and removed from the device 200 after implantation.

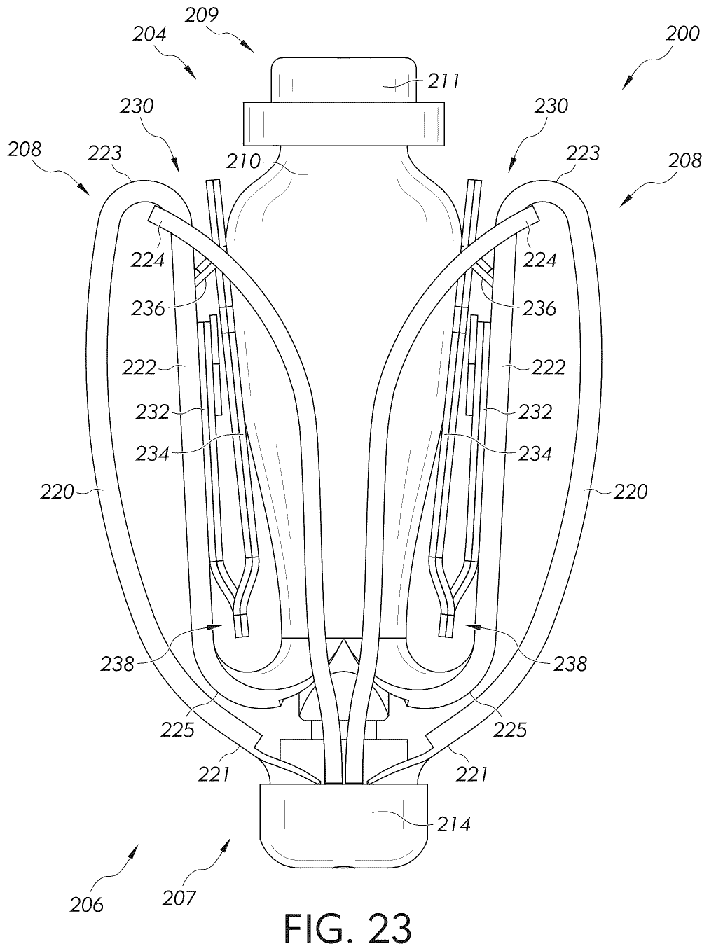

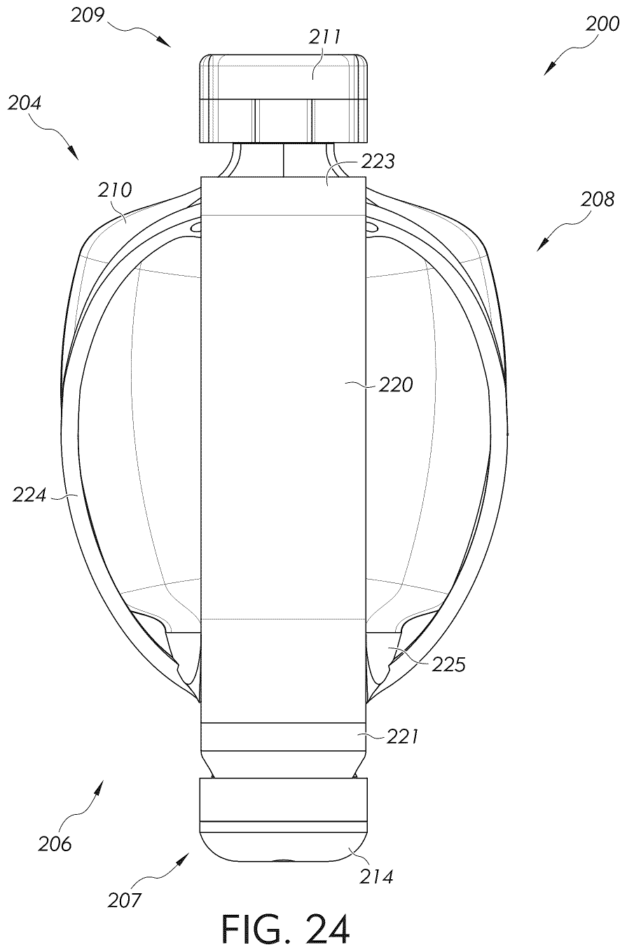

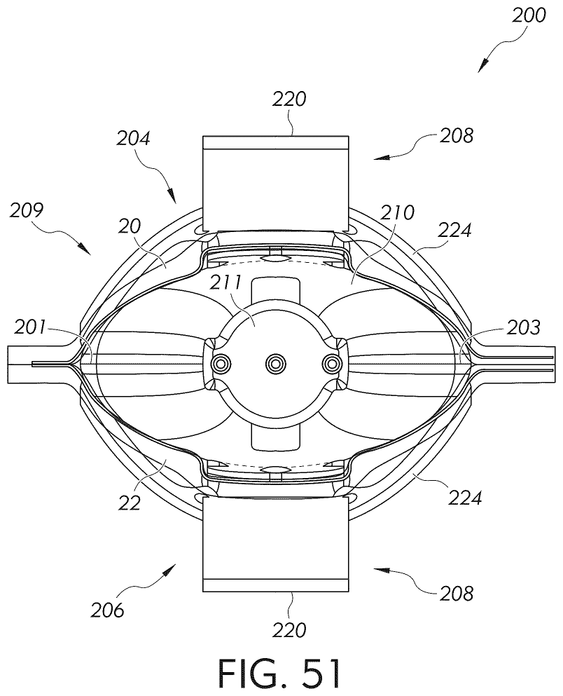

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above (e.g., FIG. 51) and has a tapered shape or cross-section when seen from a front view (e.g., FIG. 23) and a round shape or cross-section when seen from a side view (e.g., FIG. 24). A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion 232 of the clasps 230. The stiffening of the inner paddle allows the device to move to the various different positions shown and described herein. The inner paddle 222, the outer paddle 220, the coaptation can all be interconnected as described herein, such that the device 200 is constrained to the movements and positions shown and described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 provide additional pinching force between the inner paddles 222 and the coaptation element 210 and assist in wrapping the leaflets around the sides of the coaptation element 210 for a better seal between the coaptation element 210 and the leaflets, as can be seen in FIG. 51. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

Configuring the paddle frames 224 in this manner provides increased surface area compared to the outer paddles 220 alone. This can, for example, make it easier to grasp and secure the native leaflets. The increased surface area can also distribute the clamping force of the paddles 220 and paddle frames 224 against the native leaflets over a relatively larger surface of the native leaflets in order to further protect the native leaflet tissue. Referring again to FIG. 51, the increased surface area of the paddle frames 224 can also allow the native leaflets to be clamped to the device or implant 200, such that the native leaflets coapt entirely around the coaptation member or coaptation element 210. This can, for example, improve sealing of the native leaflets 20, 22 and thus prevent, inhibit, or further reduce valve regurgitation.

In some implementations the clasps comprise a movable arm coupled to the anchors. In some implementations, the clasps 230 include a base or fixed arm 232, a movable arm 234, with optional barbs, friction-enhancing elements, or securing structures 236, and a joint portion 238. The fixed arms 232 are attached to the inner paddles 222, with the joint portion 238 disposed proximate the coaptation element 210. The joint portion 238 is spring-loaded so that the fixed and movable arms 232, 234 are biased toward each other when the clasp 230 is in a closed condition. In some implementations, the clasps 230 include friction-enhancing elements or securing structures, such as barbs, protrusions, ridges, grooves, textured surfaces, adhesive, etc.

In some implementations, the fixed arms 232 are attached to the inner paddles 222 through holes or slots 231 with sutures (not shown). The fixed arms 232 can be attached to the inner paddles 222 with any suitable means, such as screws or other fasteners, crimped sleeves, mechanical latches or snaps, welding, adhesive, clamps, latches, or the like. The fixed arms 232 remain substantially stationary relative to the inner paddles 222 when the movable arms 234 are opened to open the clasps 230 and expose the optional barbs, friction-enhancing elements, or securing structures 236. The clasps 230 are opened by applying tension to actuation lines 216 (e.g., as shown in FIGS. 43-48) attached to holes 235 in the movable arms 234, thereby causing the movable arms 234 to articulate, pivot, and/or flex on the joint portions 238.

Referring now to FIG. 29, a close-up view of one of the leaflets 20, 22 grasped by a clasp such as clasp 230 is shown. The leaflet 20, 22 is grasped between the movable and fixed arms 232, 234 of the clasp 230. The tissue of the leaflet 20, 22 is not pierced by the optional barbs, friction-enhancing elements, or securing structures 236, though in some implementations the optional barbs 236 can partially or fully pierce through the leaflet 20, 22. The angle and height of the optional barbs, friction-enhancing elements or securing structures 236 relative to the movable arm 234 helps to secure the leaflet 20, 22 within the clasp 230. In particular, a force pulling the implant off of the native leaflet 20, 22 will encourage the optional barbs, friction-enhancing elements, or securing structures 236 to further engage the tissue, thereby ensuring better retention. Retention of the leaflet 20, 22 in the clasp 230 is further improved by the position of fixed arm 232 near the optional barbs, friction-enhancing elements, or securing structures 236 when the clasp 230 is closed. In this arrangement, the tissue is formed by the fixed arms 232 and the movable arms 234 and the optional barbs, friction-enhancing elements, or securing structures 236 into an S-shaped torturous path. Thus, forces pulling the leaflet 20, 22 away from the clasp 230 will encourage the tissue to further engage the optional barbs, friction-enhancing elements, or securing structures 236 before the leaflets 20, 22 can escape. For example, leaflet tension during diastole can encourage the optional barbs, friction-enhancing elements, or securing structures 236 to pull toward the end portion of the leaflet 20, 22. Thus, the S-shaped path can utilize the leaflet tension during diastole to engage the leaflets 20, 22 more tightly with the optional barbs, friction-enhancing elements or securing structures 236.

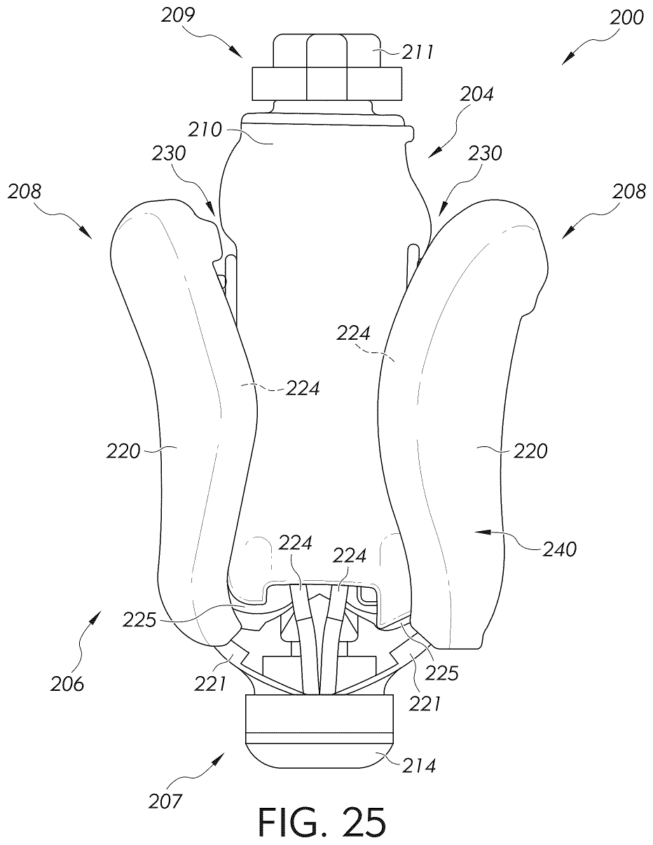

Referring to FIG. 25, the device or implant 200 can also include a cover 240. In some implementations, the cover 240 can be disposed on the coaptation element 210, the outer and inner paddles 220, 222, and/or the paddle frames 224. The cover 240 can be configured to prevent, inhibit, or reduce blood-flow through the device or implant 200 and/or to promote native tissue ingrowth. In some implementations, the cover 240 can be a cloth or fabric such as PET, velour, or other suitable fabric. In some implementations, in lieu of or in addition to a fabric, the cover 240 can include a coating (e.g., polymeric) that is applied to the device or implant 200.

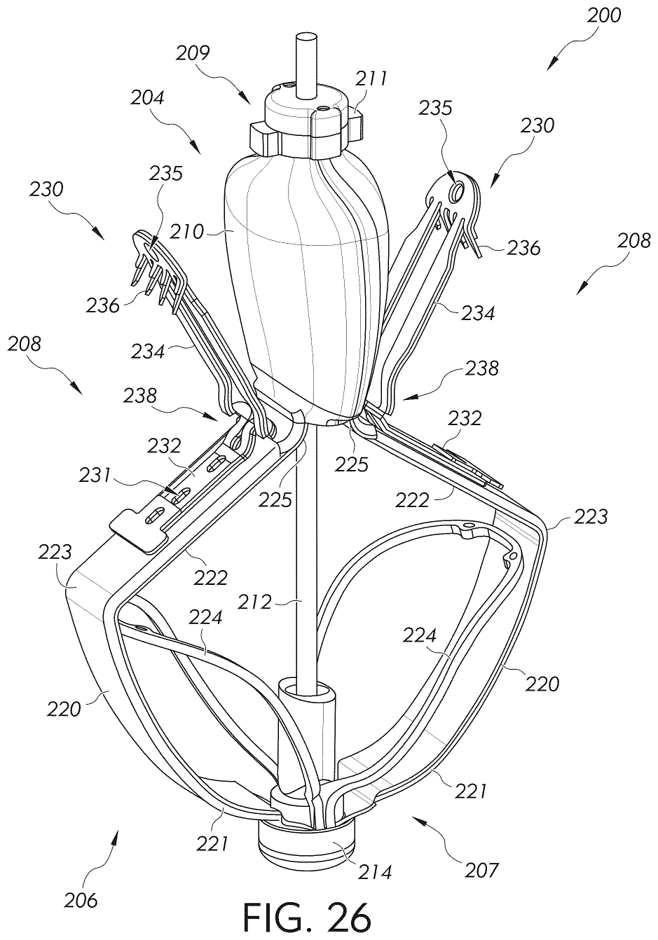

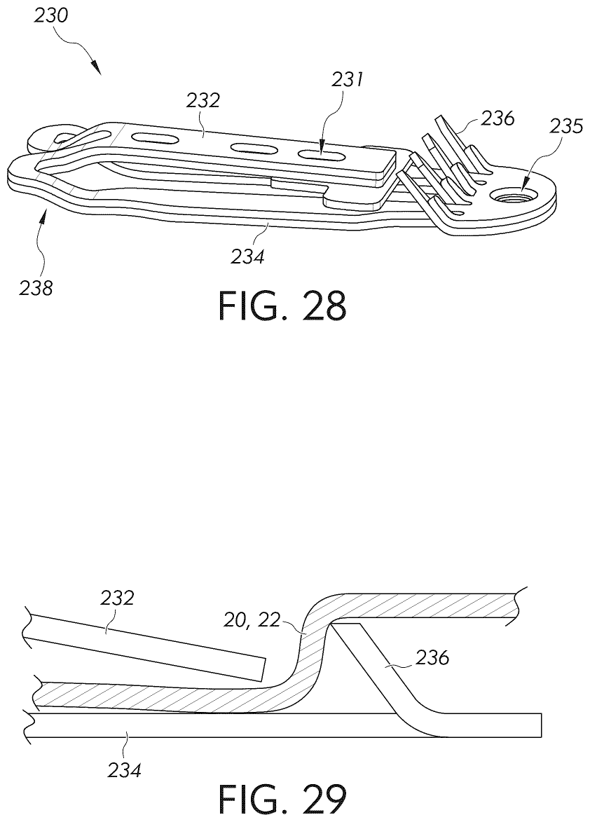

During implantation, the paddles 220, 222 of the anchors 208 are opened and closed to grasp the native valve leaflets 20, 22 between the paddles 220, 222 and the coaptation element 210. The anchors 208 are moved between a closed position (FIGS. 22-25) to various open positions (FIGS. 26-37) by extending and retracting the actuation element 212. Extending and retracting the actuation element 212 increases and decreases the spacing between the coaptation element 210 and the cap 214, respectively. The proximal collar 211 (or other attachment element) and the coaptation element 210 slide along the actuation element 212 during actuation so that changing of the spacing between the coaptation element 210 and the cap 214 causes the paddles 220, 220 to move between different positions to grasp the valve leaflets 20, 22 during implantation.

As the device 200 is opened and closed, the pair of inner and outer paddles 222, 220 are moved in unison, rather than independently, by a single actuation element 212. Also, the positions of the clasps 230 are dependent on the positions of the paddles 222, 220. For example, the clasps 230 are arranged such that closure of the anchors 208 simultaneously closes the clasps 230. In some implementations, the device 200 can be made to have the paddles 220, 222 be independently controllable in the same manner (e.g., the device 101 illustrated in FIG. 15).

In some implementations, the clasps 230 further secure the native leaflets 20, 22 by engaging the leaflets 20, 22 with optional barbs, friction-enhancing elements, or securing structures 236 and/or pinching the leaflets 20, 22 between the movable and fixed arms 234, 232. In some implementations, the clasps 230 are barbed clasps that include barbs that increase friction with and/or can partially or completely puncture the leaflets 20, 22. The actuation lines 216 (FIGS. 43-48) can be actuated separately so that each clasp 230 can be opened and closed separately. Separate operation allows one leaflet 20, 22 to be grasped at a time, or for the repositioning of a clasp 230 on a leaflet 20, 22 that was insufficiently grasped, without altering a successful grasp on the other leaflet 20, 22. The clasps 230 can be fully opened and closed when the inner paddle 222 is not closed, thereby allowing leaflets 20, 22 to be grasped in a variety of positions as the particular situation requires.

Referring now to FIGS. 22-25, the device 200 is shown in a closed position. When closed, the inner paddles 222 are disposed between the outer paddles 220 and the coaptation element 210. The clasps 230 are disposed between the inner paddles 222 and the coaptation element 210. Upon successful capture of native leaflets 20, 22 the device 200 is moved to and retained in the closed position so that the leaflets 20, 22 are secured within the device 200 by the clasps 230 and are pressed against the coaptation element 210 by the paddles 220, 222. The outer paddles 220 can have a wide curved shape that fits around the curved shape of the coaptation element 210 to grip the leaflets 20, 22 more securely when the device 200 is closed (e.g., as can be seen in FIG. 51). The curved shape and rounded edges of the outer paddle 220 also prohibits or inhibits tearing of the leaflet tissue.

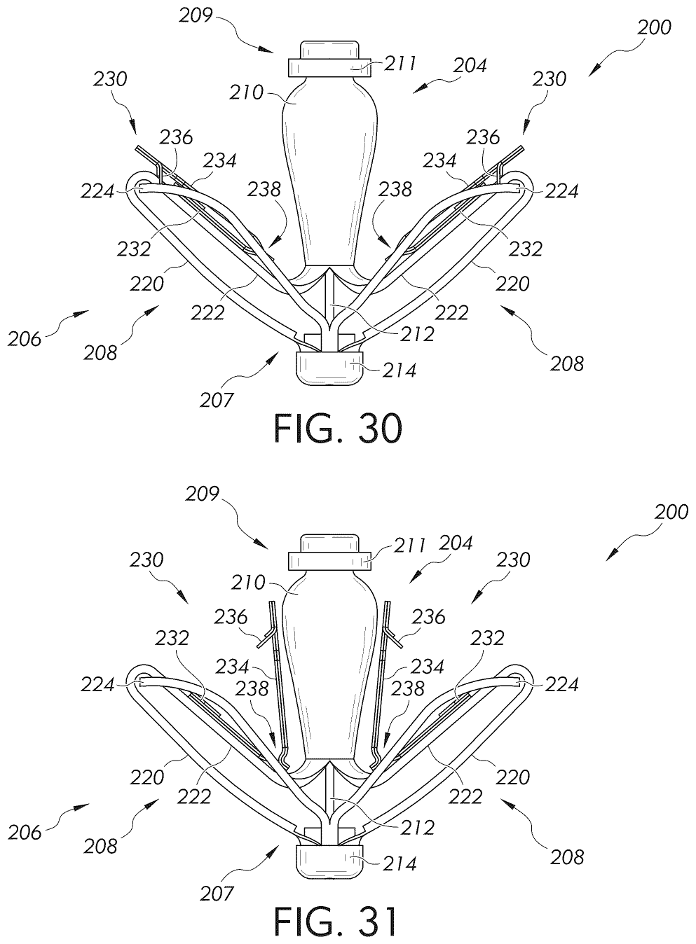

Referring now to FIGS. 30-37, the device or implant 200 described above is shown in various positions and configurations ranging from partially open to fully open. The paddles 220, 222 of the device 200 transition between each of the positions shown in FIGS. 30-37 from the closed position shown in FIGS. 22-25 up extension of the actuation element 212 from a fully retracted to fully extended position.

Referring now to FIGS. 30-31, the device 200 is shown in a partially open position. The device 200 is moved into the partially open position by extending the actuation element 212. Extending the actuation element 212 pulls down on the bottom portions of the outer paddles 220 and paddle frames 224. The outer paddles 220 and paddle frames 224 pull down on the inner paddles 222, where the inner paddles 222 are connected to the outer paddles 220 and the paddle frames 224. Because the proximal collar 211 (or other attachment element) and coaptation element 210 are held in place by the capture mechanism 213, the inner paddles 222 are caused to articulate, pivot, and/or flex in an opening direction. The inner paddles 222, the outer paddles 220, and the paddle frames all flex to the position shown in FIGS. 30-31. Opening the paddles 222, 220 and frames 224 forms a gap between the coaptation element 210 and the inner paddle 222 that can receive and grasp the native leaflets 20, 22. This movement also exposes the clasps 230 that can be moved between closed (FIG. 30) and open (FIG. 31) positions to form a second gap for grasping the native leaflets 20, 22. The extent of the gap between the fixed and movable arms 232, 234 of the clasp 230 is limited to the extent that the inner paddle 222 has spread away from the coaptation element 210.

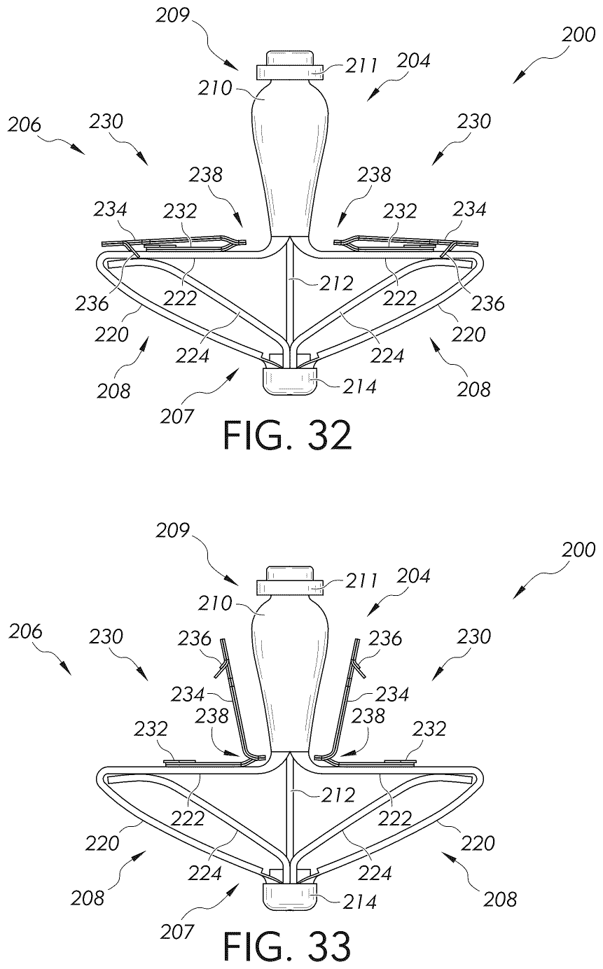

Referring now to FIGS. 32-33, the device 200 is shown in a laterally extended or open position. The device 200 is moved into the laterally extended or open position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the laterally extended or open position, the inner paddles 222 extend horizontally more than in other positions of the device 200 and form an approximately 90-degree angle with the coaptation element 210. Similarly, the paddle frames 224 are at their maximum spread position when the device 200 is in the laterally extended or open position. The increased gap between the coaptation element 210 and inner paddle 222 formed in the laterally extended or open position allows clasps 230 to open further (FIG. 33) before engaging the coaptation element 210, thereby increasing the size of the gap between the fixed and movable arms 232, 234.

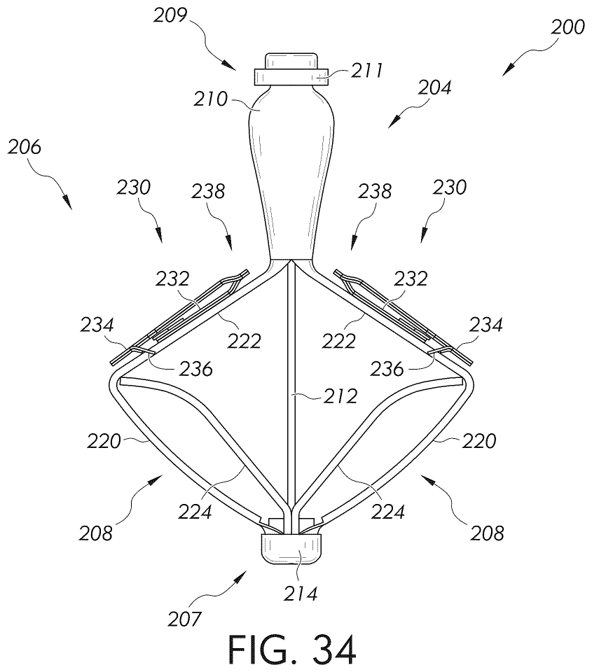

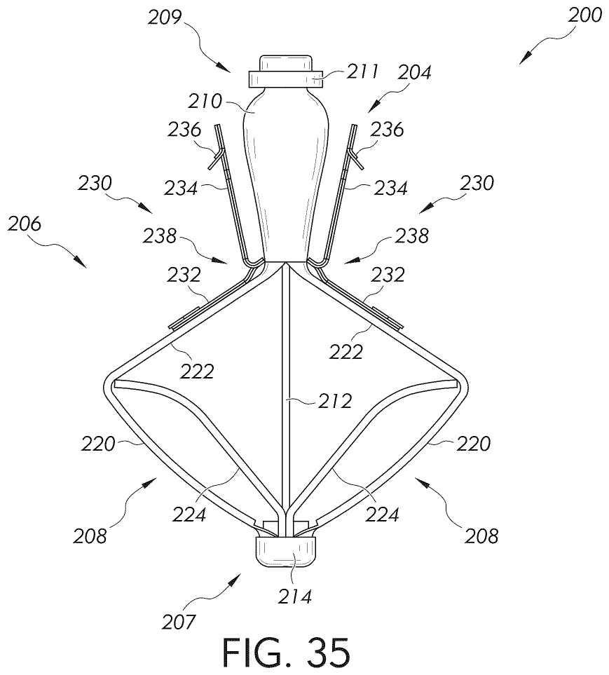

Referring now to FIGS. 34-35, the example device 200 is shown in a three-quarters extended position. The device 200 is moved into the three-quarters extended position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the three-quarters extended position, the inner paddles 222 are open beyond 90 degrees to an approximately 135-degree angle with the coaptation element 210. The paddle frames 224 are less spread than in the laterally extended or open position and begin to move inward toward the actuation element 212 as the actuation element 212 extends further. The outer paddles 220 also flex back toward the actuation element 212. As with the laterally extended or open position, the increased gap between the coaptation element 210 and inner paddle 222 formed in the laterally extended or open position allows clasps 230 to open even further (FIG. 35), thereby increasing the size of the gap between the fixed and movable arms 232, 234.

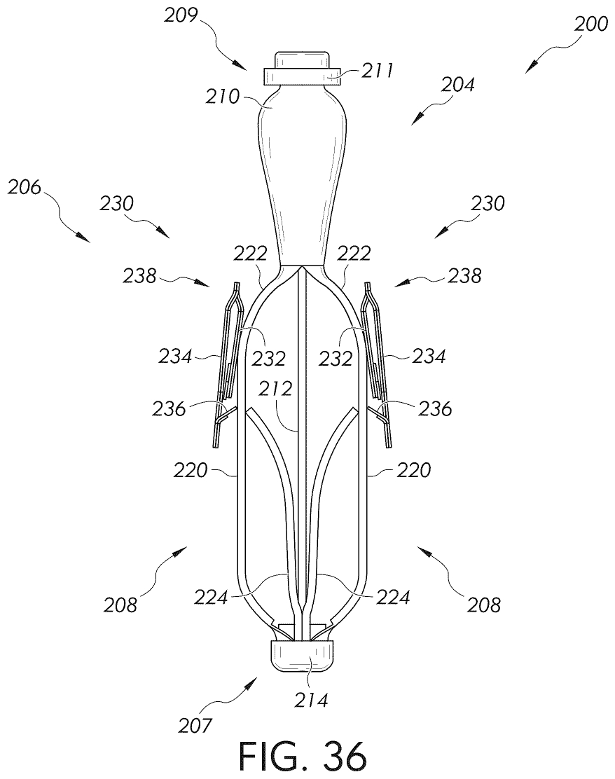

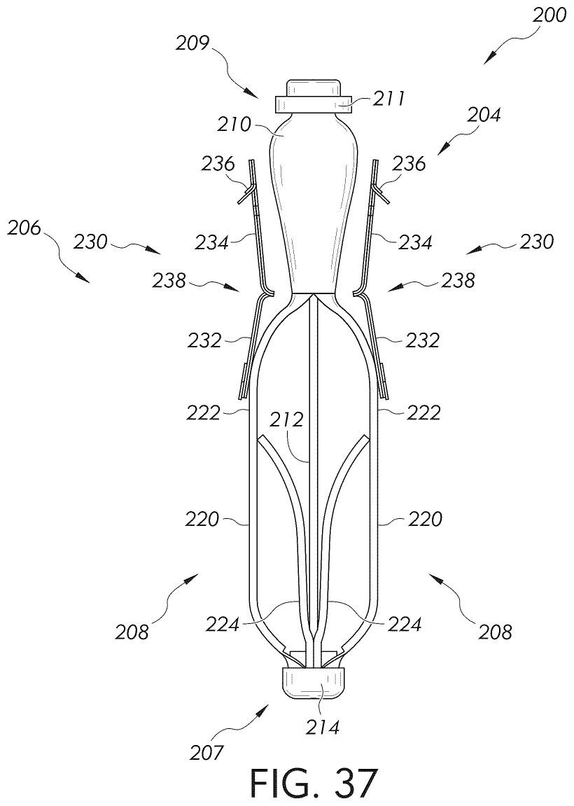

Referring now to FIGS. 36-37, the example device 200 is shown in a fully extended position. The device 200 is moved into the fully extended position by continuing to extend the actuation element 212 described above, thereby increasing the distance between the coaptation element 210 and the cap 214 of the distal portion 207 to a maximum distance allowable by the device 200. Continuing to extend the actuation element 212 pulls down on the outer paddles 220 and paddle frames 224, thereby causing the inner paddles 222 to spread apart further from the coaptation element 210. The outer paddles 220 and paddle frames 224 move to a position where they are close to the actuation element. In the fully extended position, the inner paddles 222 are open to an approximately 180-degree angle with the coaptation element 210. The inner and outer paddles 222, 220 are stretched straight in the fully extended position to form an approximately 180-degree angle between the paddles 222, 220. The fully extended position of the device 200 provides the maximum size of the gap between the coaptation element 210 and inner paddle 222, and, in some implementations, allows clasps 230 to also open fully to approximately 180 degrees (FIG. 37) between the fixed and movable arms 232, 234 of the clasp 230. The position of the device 200 is the longest and the narrowest configuration. Thus, the fully extended position of the device 200 can be a desirable position for bailout of the device 200 from an attempted implantation or can be a desired position for placement of the device in a delivery catheter, or the like.

Configuring the device or implant 200 such that the anchors 208 can extend to a straight or approximately straight configuration (e.g., approximately 120-180 degrees relative to the coaptation element 210) can provide several advantages. For example, this configuration can reduce the radial crimp profile of the device or implant 200. It can also make it easier to grasp the native leaflets 20, 22 by providing a larger opening between the coaptation element 210 and the inner paddles 222 in which to grasp the native leaflets 20, 22. Additionally, the relatively narrow, straight configuration can prevent or reduce the likelihood that the device or implant 200 will become entangled in native anatomy (e.g., chordae tendineae CT shown in FIGS. 3 and 4) when positioning and/or retrieving the device or implant 200 into the delivery system 202.

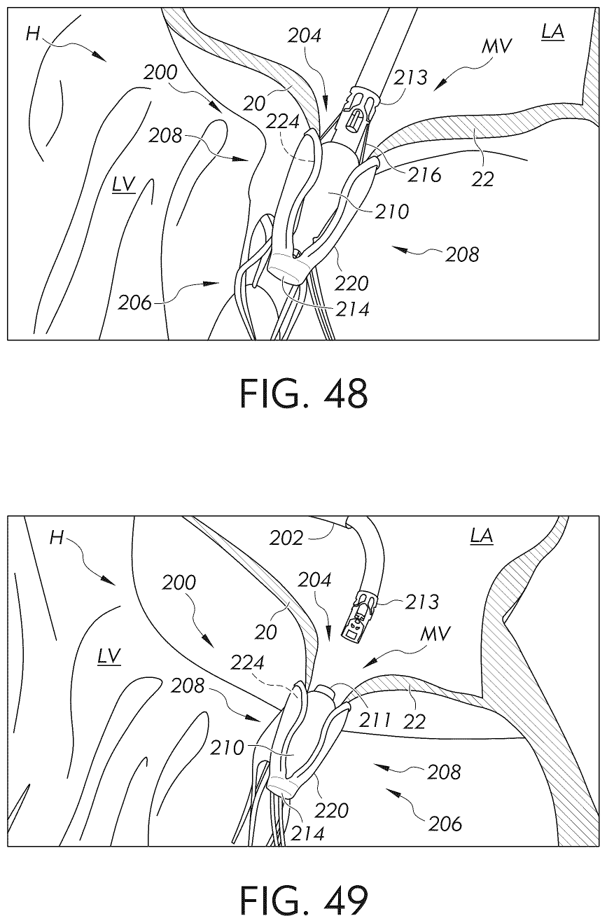

Referring now to FIGS. 38-49, an example device 200 is shown being delivered and deployed within the native mitral valve MV of the heart H. As described above, the device 200 shown in FIGS. 38-49 includes the optional covering 240 (e.g., FIG. 25) over the coaptation element 210, clasps 230, inner paddles 222 and/or the outer paddles 220. The device 200 is deployed from a delivery system 202 (e.g., which can comprise a device/implant catheter that is extendable from a steerable catheter and/or a guide sheath) and is retained by a capture mechanism 213 (see e.g., FIGS. 43 and 48) and is actuated by extending or retracting the actuation element 212. Fingers of the capture mechanism 213 removably attach the collar 211 to the delivery system 202. In some implementations, the capture mechanism 213 is held closed around the collar 211 by the actuation element 212, such that removal of the actuation element 212 allows the fingers of the capture mechanism 213 to open and release the collar 211 to decouple the capture mechanism 213 from the device 200 after the device 200 has been successfully implanted.

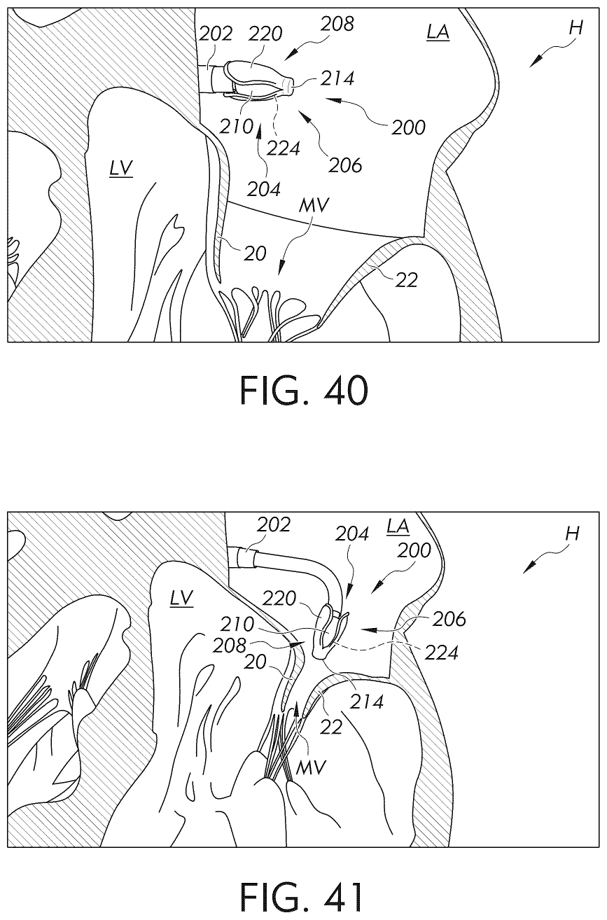

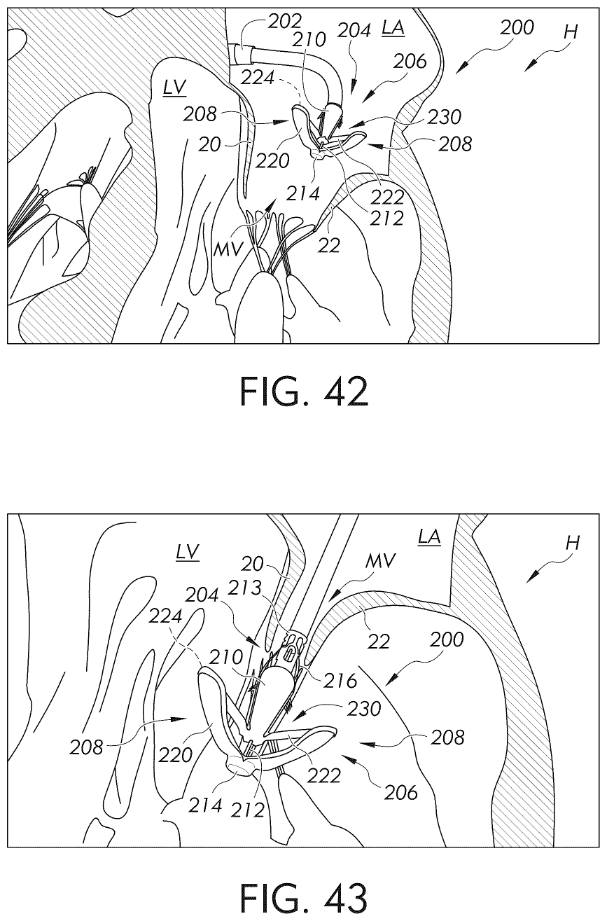

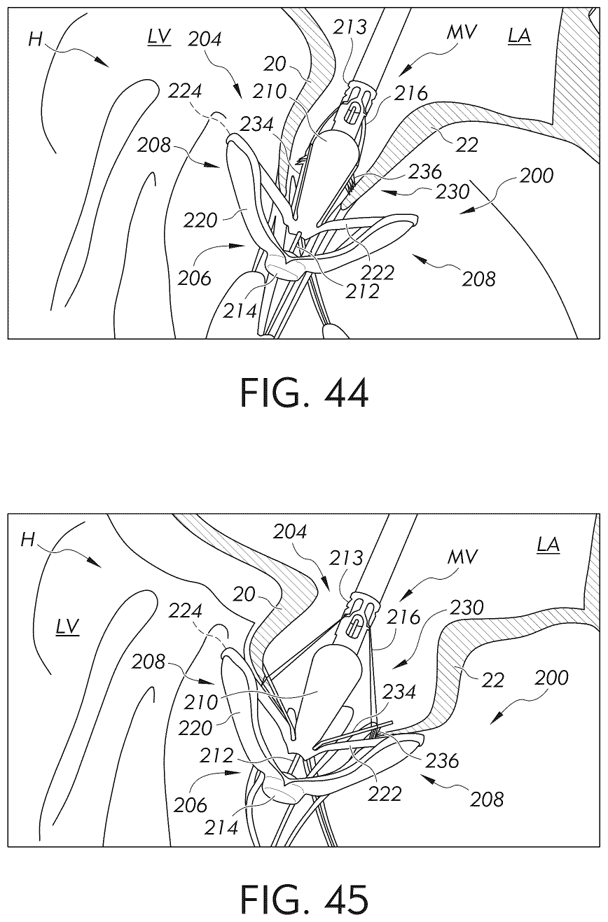

Referring now to FIG. 38, the delivery system 202 (e.g., a delivery catheter/sheath thereof) is inserted into the left atrium LA through the septum and the device/implant 200 is deployed from the delivery system 202 (e.g., a device/implant catheter retaining the device/implant can be extended to deploy the device/implant out from a steerable catheter) in the fully open condition for the reasons discussed above with respect to the device 100. The actuation element 212 is then retracted to move the device 200 through the partially closed condition (FIG. 39) and to the fully closed condition shown in FIGS. 40-41. Then the delivery system or catheter maneuvers the device/implant 200 towards the mitral valve MV as shown in FIG. 41. Referring now to FIG. 42, when the device 200 is aligned with the mitral valve MV, the actuation element 212 is extended to open the paddles 220, 222 into the partially opened position and the actuation lines 216 (FIGS. 43-48) are retracted to open the clasps 230 to prepare for leaflet grasp. Next, as shown in FIGS. 43-44, the partially open device 200 is inserted through the native valve (e.g., by advancing a device/implant catheter from a steerable catheter) until leaflets 20, 22 are properly positioned in between the inner paddles 222 and the coaptation element 210 and inside the open clasps 230.

FIG. 45 shows the device 200 with both clasps 230 closed, though the optional barbs, friction-enhancing elements, or securing structures 236 of one clasp 230 missed one leaflet 22. As can be seen in FIGS. 45-47, the out of position clasp 230 is opened and closed again to properly grasp the missed leaflet 22. When both leaflets 20, 22 are grasped properly, the actuation element 212 is retracted to move the device 200 into the fully closed position shown in FIG. 48. With the device 200 fully closed and implanted in the native valve, the actuation element 212 is disengaged from the cap 214 and is withdrawn to release the capture mechanism 213 from the proximal collar 211 (or other attachment element) so that the capture mechanism 213 can be withdrawn into the delivery system 202 (e.g., into a catheter/sheath), as shown in FIG. 49. Once deployed, the device 200 can be maintained in the fully closed position with a mechanical means such as a latch or can be biased to remain closed through the use of spring material, such as steel, and/or shape-memory alloys such as Nitinol. For example, the paddles 220, 222 can be formed of steel or Nitinol shape-memory alloy—produced in a wire, sheet, tubing, or laser sintered powder—and are biased to hold the outer paddles 220 closed around the inner paddles 222, coaptation element 210, and/or the clasps 230 pinched around native leaflets 20, 22.

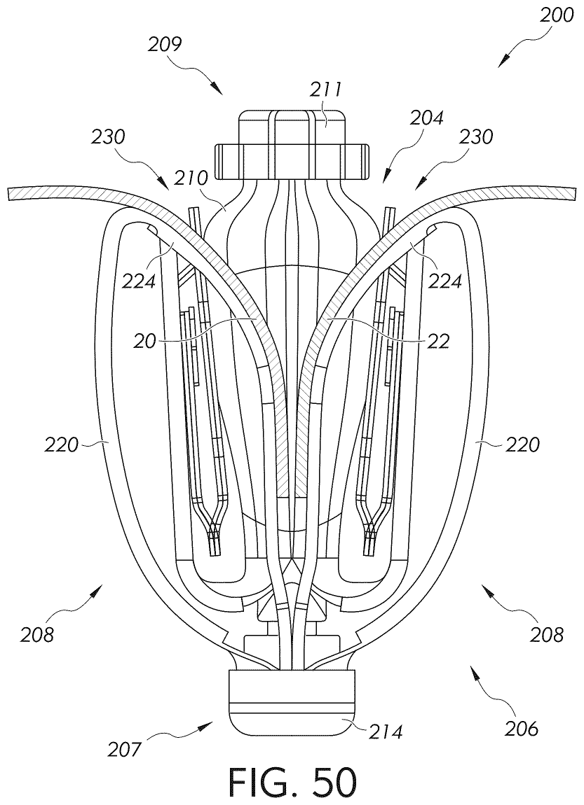

Referring to FIGS. 50-54, once the device 200 is implanted in a native valve, the coaptation element 210 functions as a gap filler in the valve regurgitant orifice, such as the gap 26 in the mitral valve MV illustrated by FIG. 6 or a gap in another native valve. In some implementations, when the device 200 has been deployed between the two opposing valve leaflets 20, 22, the leaflets 20, 22 no longer coapt against each other in the area of the coaptation element 210, but instead coapt against the coaptation element 210. This reduces the distance the leaflets 20, 22 need to be approximated to close the native valve during systole, thereby facilitating repair of functional valve disease that may be causing valvular regurgitation. A reduction in leaflet approximation distance can result in several other advantages as well. For example, the reduced approximation distance required of the leaflets 20, 22 reduces or minimizes the stress experienced by the native valve. Shorter approximation distance of the valve leaflets 20,22 can also require less approximation forces which can result in less tension experienced by the leaflets 20, 22 and less diameter reduction of the valve annulus. The smaller reduction of the valve annulus—or none at all—can result in less reduction in valve orifice area as compared to a device without a coaptation element or spacer. In this way, the coaptation element 210 can reduce the transvalvular gradients.

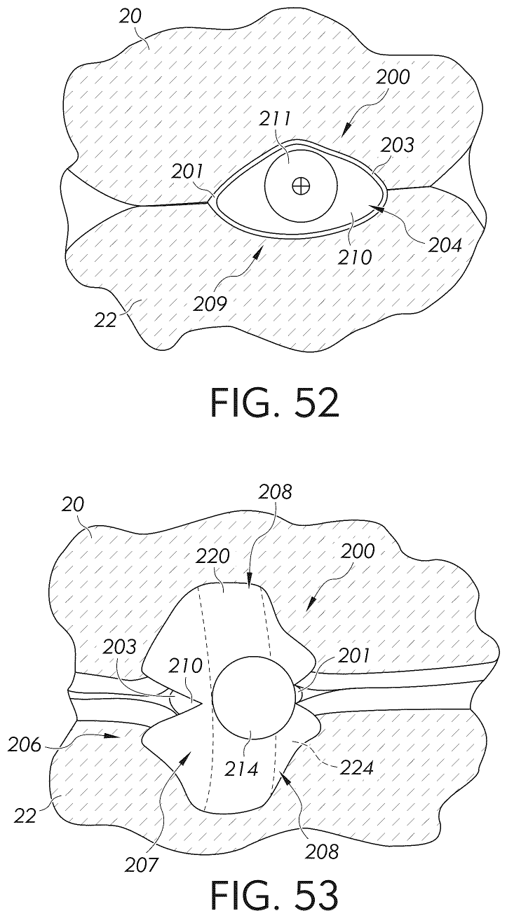

To adequately fill the gap 26 between the leaflets 20, 22, the device 200 and the components thereof can have a wide variety of different shapes and sizes. For example, the outer paddles 220 and paddle frames 224 can be configured to conform to the shape or geometry of the coaptation element 210 as is shown in FIGS. 50-54. As a result, the outer paddles 220 and paddle frames 224 can mate with both the coaptation element 210 and the native valve leaflets 20, 22. In some implementations, when the leaflets 20, 22 are coapted against the coaptation element 210, the leaflets 20, 22 fully surround or “hug” the coaptation element 210 in its entirety, thus small leaks at lateral and medial aspects 201, 203 of the coaptation element 210 can be prevented or inhibited. The interaction of the leaflets 20, 22 and the device 200 is made clear in FIG. 51, which shows a schematic atrial or surgeon's view that shows the paddle frame 224 (which would not actually be visible from a true atrial view, e.g., FIG. 52), conforming to the coaptation element 210 geometry. The opposing leaflets 20, 22 (the ends of which would also not be visible in the true atrial view, e.g., FIG. 52) being approximated by the paddle frames 224, to fully surround or “hug” the coaptation element 210.

This coaptation of the leaflets 20, 22 against the lateral and medial aspects 201, 203 of the coaptation element 210 (shown from the atrial side in FIG. 52, and the ventricular side in FIG. 53) would seem to contradict the statement above that the presence of a coaptation element 210 minimizes the distance the leaflets need to be approximated. However, the distance the leaflets 20, 22 need to be approximated is still minimized if the coaptation element 210 is placed precisely at a regurgitant gap 26 and the regurgitant gap 26 is less than the width (medial-lateral) of the coaptation element 210.

FIG. 50 illustrates the geometry of the coaptation element 210 and the paddle frame 224 from an LVOT perspective. As can be seen in this view, the coaptation element 210 has a tapered shape being smaller in dimension in the area closer to where the inside surfaces of the leaflets 20, 22 are required to coapt and increase in dimension as the coaptation element 210 extends toward the atrium. Thus, the depicted native valve geometry is accommodated by a tapered coaptation element geometry. Still referring to FIG. 50, the tapered coaptation element geometry, in conjunction with the illustrated expanding paddle frame 224 shape (toward the valve annulus) can help to achieve coaptation on the lower end of the leaflets, reduce stress, and minimize transvalvular gradients.

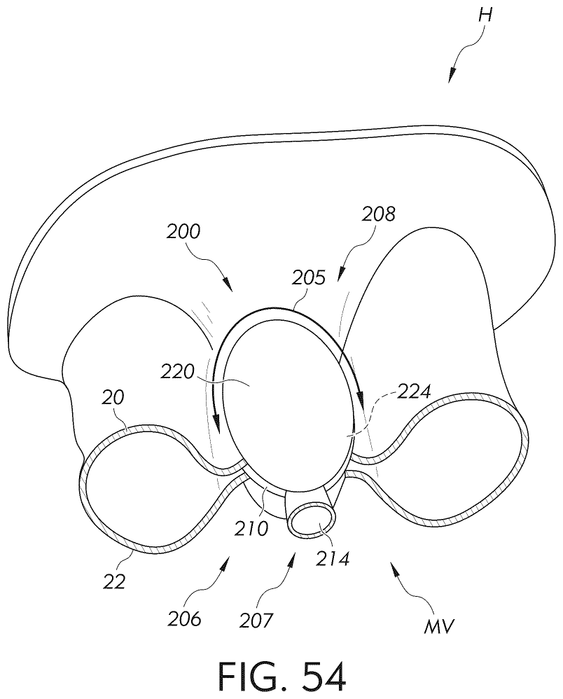

Referring to FIG. 54, the shape of the coaptation element 210 and the paddle frames 224 can be defined based on an Intra-Commissural view of the native valve and the device 200. Two factors of these shapes are leaflet coaptation against the coaptation element 210 and reduction of stress on the leaflets due to the coaptation. Referring to FIGS. 54 and 24, to both coapt the valve leaflets 20, 22 against the coaptation element 210 and reduce the stress applied to the valve leaflets 20, 22 by the coaptation element 210 and/or the paddle frames 224, the coaptation element 210 can have a round or rounded shape and the paddle frames 224 can have a full radius that spans nearly the entirety of the paddle frame 224. The round shape of the coaptation element 210 and/or the illustrated fully rounded shape of the paddle frames 224 distributes the stresses on the leaflets 20, 22 across a large, curved engagement area 205. For example, in FIG. 54, the force on the leaflets 20, 22 by the paddle frames is spread along the entire rounded length of the paddle frame 224, as the leaflets 20 try to open during the diastole cycle.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in their entirety for all purposes.

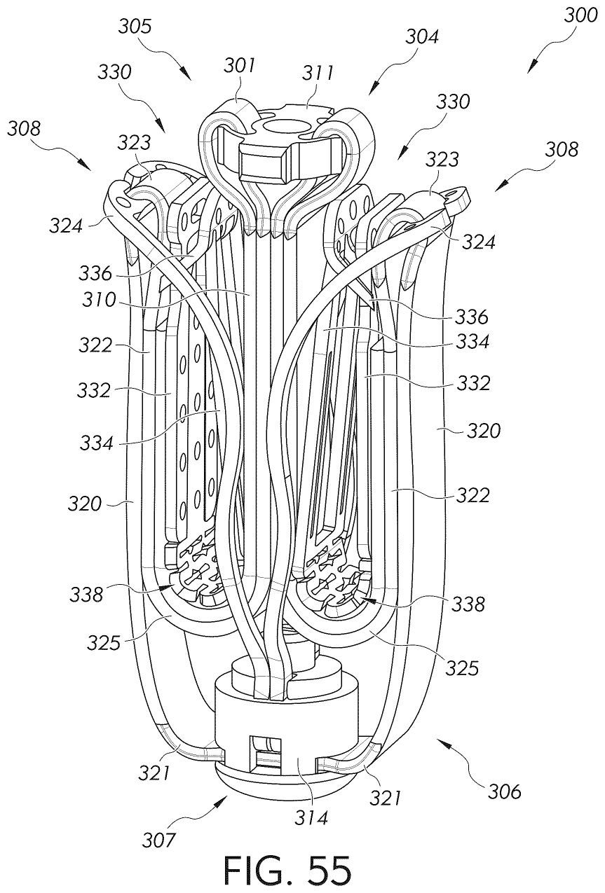

Referring now to FIG. 55, an example of a device or implant 300 (e.g., an implantable prosthetic device, a prosthetic spacer device, a valve repair device, a repair device, a treatment device, etc.) is shown. The device 300 is one of the many different configurations that the device 100 that is schematically illustrated in FIGS. 8-14 can take. The device 300 can include any other features for a device or implant discussed in the present application, and the device 300 can be positioned to engage valve tissue 20, 22 as part of any suitable valve repair system (e.g., any valve repair system disclosed in the present application).

The device or implant 300 includes a proximal or attachment portion 305, an anchor portion 306, and a distal portion 307. In some implementations, the device/implant 300 includes a coaptation portion/region 304, and the coaptation portion/region 304 can optionally include a coaptation element 310 (e.g., spacer, plug, membrane, sheet, gap filler, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism (e.g., a capture mechanism such as the capture mechanism 213 shown in FIGS. 43-49, or another capture mechanism described herein or otherwise known) of a delivery system (e.g., a delivery system such as the system shown in FIGS. 38-42 and 49).

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation member or coaptation element 310, the coaptation member or coaptation element 310 and the anchors 308 can be coupled together in various ways. For example, as shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device. For example, the anchors 308 can be positioned in a fully extended or straight configuration (e.g., similar to the configuration of device 200 shown in FIG. 36) by moving the distal end (e.g., cap 314, etc.) away from the proximal end of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the coaptation element 310 (e.g., similar to the configuration of device 200 shown in FIG. 36). From the straight configuration, the anchors 308 can be moved to a fully folded configuration (e.g., FIG. 55), e.g., by moving the proximal end and distal end toward each other and/or toward a midpoint or center of the device. Initially, as the distal end (e.g., cap 314, etc.) moves toward the proximal end and/or midpoint or center of the device, the anchors 308 bend at connection portions 321, 323, 325, and the connection portions 323 move radially outwardly relative to the longitudinal axis of the device 300 and axially toward the midpoint and/or toward the proximal end of the device (e.g., similar to the configuration of device 200 shown in FIG. 34). As the cap 314 continues to move toward the midpoint and/or toward the proximal end of the device, the connection portions 323 move radially inwardly relative to the longitudinal axis of the device 300 and axially toward the proximal end of the device (e.g., similar to the configuration of device 200 shown in FIG. 30).

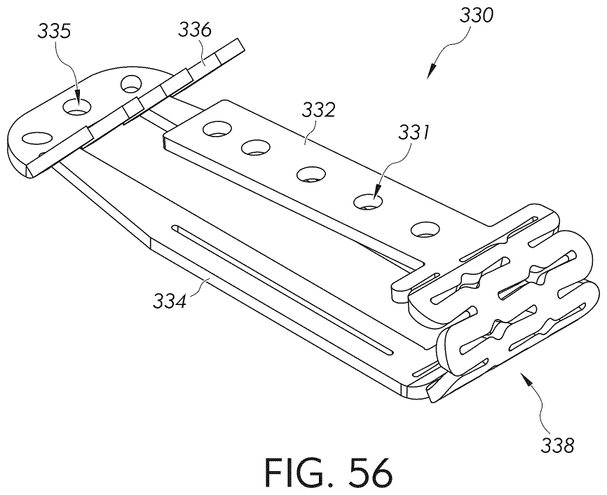

In some implementations, the clasps comprise a movable arm coupled to an anchor. In some implementations, the clasps 330 (as shown in detail in FIG. 56) include a base or fixed arm 332, a movable arm 334 with optional barbs, friction enhancing elements, or securing structures 336, and a joint portion 338. The fixed arms 332 are attached to the inner paddles 322, with the joint portion 338 disposed proximate the coaptation element 310. The joint portion 338 is spring-loaded so that the fixed and movable arms 332, 334 are biased toward each other when the clasp 330 is in a closed condition.

The fixed arms 332 are attached to the inner paddles 322 through holes or slots 331 with sutures (not shown). The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the movable arms 334 are opened to open the clasps 330 and expose the optional barbs, friction-enhancing elements, or securing structures 336. The clasps 330 are opened by applying tension to actuation lines (e.g., the actuation lines 216 shown in FIGS. 43-48) attached to holes 335 in the movable arms 334, thereby causing the movable arms 334 to articulate, pivot, and/or flex on the joint portions 338.

In short, the device or implant 300 is similar in configuration and operation to the device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 55 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 55, the strip of material 301 begins and ends in the location of the inner paddles 322.

As with the device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which can be wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety for all purposes.

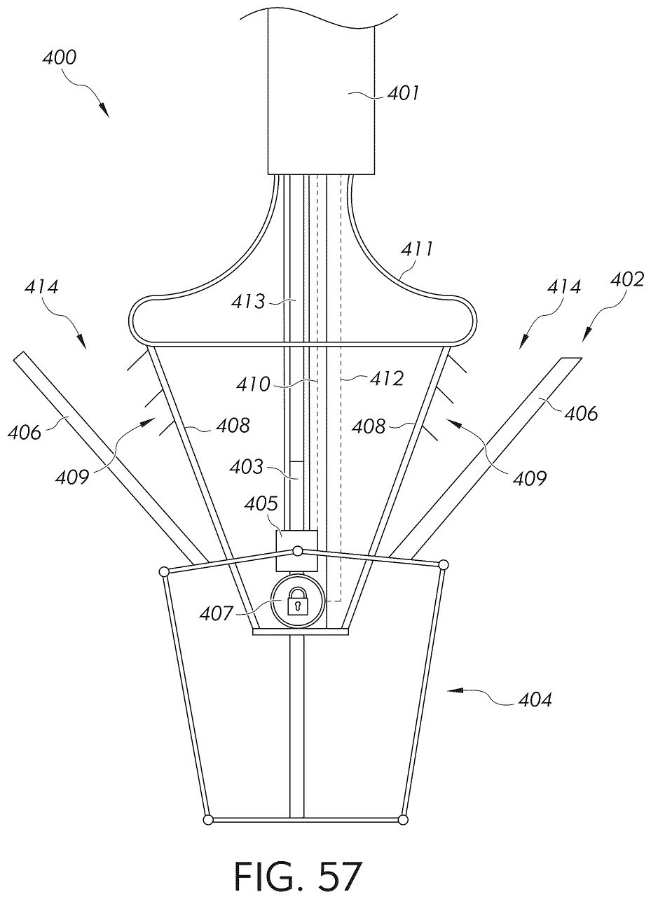

The concepts disclosed by the present application can be used with a wide variety of different devices or valve repair devices, including implantable and non-implantable ones. FIGS. 57-63 illustrate another example of one of the many valve repair systems 400 for repairing a native valve of a patient that the concepts of the present application can be applied to. The valve repair system 400 includes a delivery device 401 and a device 402 (illustrated here, for example, as a valve repair device or valve treatment device).

In some implementations, the valve repair device or treatment device 402 includes a base assembly 404, a pair of paddles 406, and a pair of gripping members 408 (e.g., clasps, clasp arms, grippers, gripping arms, latches, etc.). In some implementations, the paddles 406 can be integrally formed with the base assembly. For example, the paddles 406 can be formed as extensions of links of the base assembly. In the illustrated example, the base assembly 404 of the valve repair device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include a gripping portion 409 (e.g., barbs, protrusions, ridges, grooves, textured surfaces, adhesive, etc.) for attaching the gripping members to valve tissue when the valve repair device 402 is attached to the valve tissue. The gripping member 408 forms a means for gripping the valve tissue (in particular tissue of the valve leaflets) with a sticking means or portion such as the gripping portion 409. When the paddles 406 are in the closed position, the paddles engage the gripping members 408, such that, when valve tissue is attached to the gripping portion 409 of the gripping members, the paddles act as holding or securing means to hold the valve tissue at the gripping members and to secure the valve repair device 402 to the valve tissue. In some implementations, the gripping members 408 are configured to engage the paddles 406 such that the gripping portion 409 engages the valve tissue member and the paddles 406 to secure the valve repair device 402 to the valve tissue member. For example, in certain situations, it can be advantageous to have the paddles 406 maintain an open position and have the gripping members 408 move outward toward the paddles 406 to engage valve tissue and the paddles 406.

While the implementations shown in FIGS. 57-63 illustrate a pair of paddles 406 and a pair of gripping members 408, it should be understood that the valve repair device 402 can include any suitable number of paddles and gripping members.

In some implementations, the valve repair system 400 includes a placement shaft 413 that is removably attached to the shaft 403 of the base assembly 404 of the valve repair device 402. After the valve repair device 402 is secured to valve tissue, the placement shaft 413 is removed from the shaft 403 to remove the valve repair device 402 from the remainder of the valve repair system 400, such that the valve repair device 402 can remain attached to the valve tissue, and the delivery device 401 can be removed from a patient's body.

The valve repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture, a wire, a rod, a catheter, a tube, a hypotube, etc.

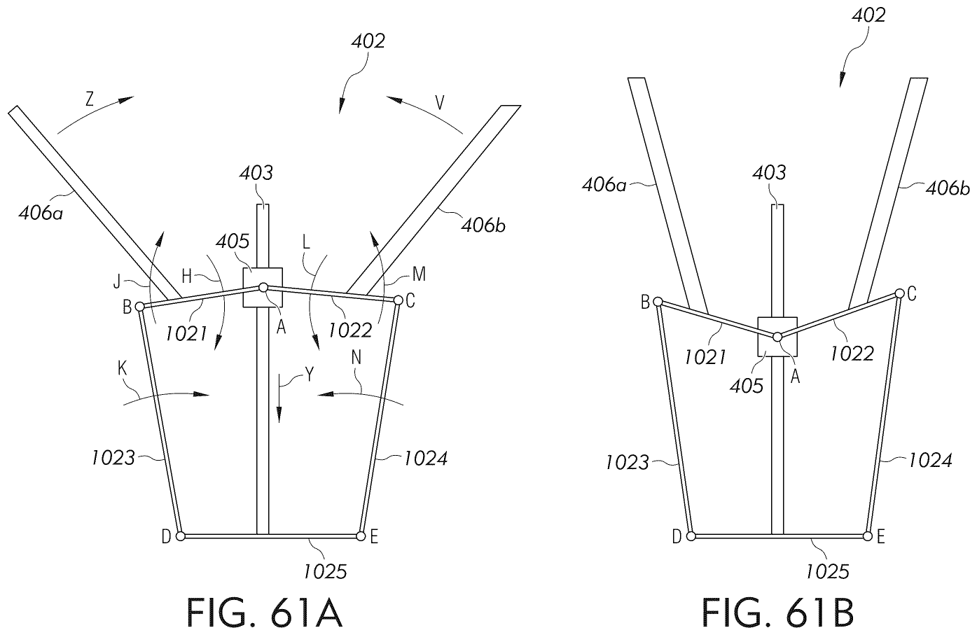

The lock control mechanism 412 is configured to lock and unlock the lock. The lock 407 serves as a locking means for locking the coupler 405 in a stationary position with respect to the shaft 403 and can take a wide variety of different forms and the type of lock control mechanism 412 can be dictated by the type of lock used. In some implementations the lock 407 includes a pivotable plate having a hole, in which the shaft 403 of the valve repair device 402 is disposed within the hole of the pivotable plate. In this implementation, when the pivotable plate is in the tilted position, the pivotable plate engages the shaft 403 to maintain a position on the shaft 403, but, when the pivotable plate is in a substantially non-tilted position, the pivotable plate can be moved along the shaft (which allows the coupler 405 to move along the shaft 403). In other words, the coupler 405 is prevented or inhibited from moving in the direction Y (as shown in FIG. 61A) along the shaft 403 when the pivotable plate of the lock 407 is in a tilted (or locked) position, and the coupler is allowed to move in the direction Y along the shaft 403 when the pivotable plate is in a substantially non-tilted (or unlocked) position. In implementations in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position. In some implementations, the pivotable plate of the lock 407 is biased in the tilted (or locked) position, and the lock control mechanism 412 is used to move the plate from the tilted position to the substantially non-tilted (or unlocked) position. In some implementations, the pivotable plate of the lock 407 is biased in the substantially non-tilted (or unlocked) position, and the lock control mechanism 412 is used to move the plate from the substantially non-tilted position to the tilted (or locked) position.

FIGS. 61A-61B illustrate the valve repair device or treatment device 402 moving from an open position (as shown in FIG. 61A) to a closed position (as shown in FIG. 61B). The base assembly 404 includes a first link 1021 extending from point A to point B, a second link 1022 extending from point A to point C, a third link 1023 extending from point B to point D, a fourth link 1024 extending from point C to point E, and a fifth link 1025 extending from point D to point E. The coupler 405 is movably attached to the shaft 403, and the shaft 403 is fixed to the fifth link 1025. The first link 1021 and the second link 1022 are pivotally attached to the coupler 405 at point A, such that movement of the coupler 405 along the shaft 403 moves the location of point A and, consequently, moves the first link 1021 and the second link 1022. The first link 1021 and the third link 1023 are pivotally attached to each other at point B, and the second link 1022 and the fourth link 1024 are pivotally attached to each other at point C. One paddle 406a is attached to first link 1021 such that movement of first link 1021 causes the paddle 406a to move, and the other paddle 406b is attached to the second link 1022 such that movement of the second link 1022 causes the paddle 406b to move. In some implementations, the paddles 406a, 406b can be connected to links 1023, 1024 or be extensions of links 1023, 1024.

In order to move the valve repair device from the open position (as shown in FIG. 61A) to the closed position (as shown in FIG. 61B), the coupler 405 is moved along the shaft 403 in the direction Y, which moves the pivot point A for the first link 1021 and the second link 1022 to a new position. Movement of the coupler 405 (and pivot point A) in the direction Y causes a portion of the first link 1021 near point A to move in the direction H, and the portion of the first link 1021 near point B to move in the direction J. The paddle 406a is attached to the first link 1021 such that movement of the coupler 405 in the direction Y causes the paddle 406a to move in the direction Z. In addition, the third link 1023 is pivotally attached to the first link 1021 at point B such that movement of the coupler 405 in the direction Y causes the third link 1023 to move in the direction K. Similarly, movement of the coupler 405 (and pivot point A) in the direction Y causes a portion of the second link 1022 near point A to move in the direction L, and the portion of the second link 1022 near point C to move in the direction M. The paddle 406b is attached to the second link 1022 such that movement of the coupler 405 in the direction Y causes the paddle 406b to move in the direction V. In addition, the fourth link 1024 is pivotally attached to the second link 1022 at point C such that movement of the coupler 405 in the direction Y causes the fourth link 1024 to move in the direction N. FIG. 61B illustrates the final position of the valve repair device 402 after the coupler 405 is moved as shown in FIG. 61A.

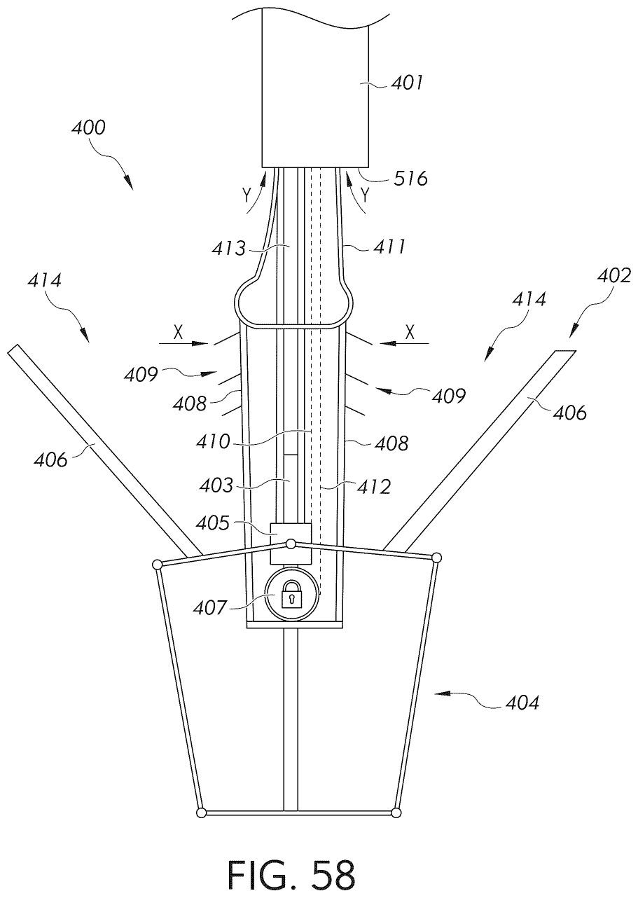

Referring to FIG. 58, the valve repair device 402 is shown in the open position (similar to the position shown in FIG. 61A), and the gripper control mechanism 411 is shown moving the gripping members 408 to provide a wider gap at the opening 414 between the gripping members and the paddles 406. In the illustrated example, the gripper control mechanism 411 includes a line, such as a suture, a wire, etc. that is threaded through an opening in an end of the gripping members 408. Both ends of the line extend through the delivery opening 516 of the delivery device 401. When the line is pulled through the delivery opening 516 in the direction Y, the gripping members 408 move inward in the direction X, which causes the opening 414 between the gripping members and the paddles 406 to become wider.

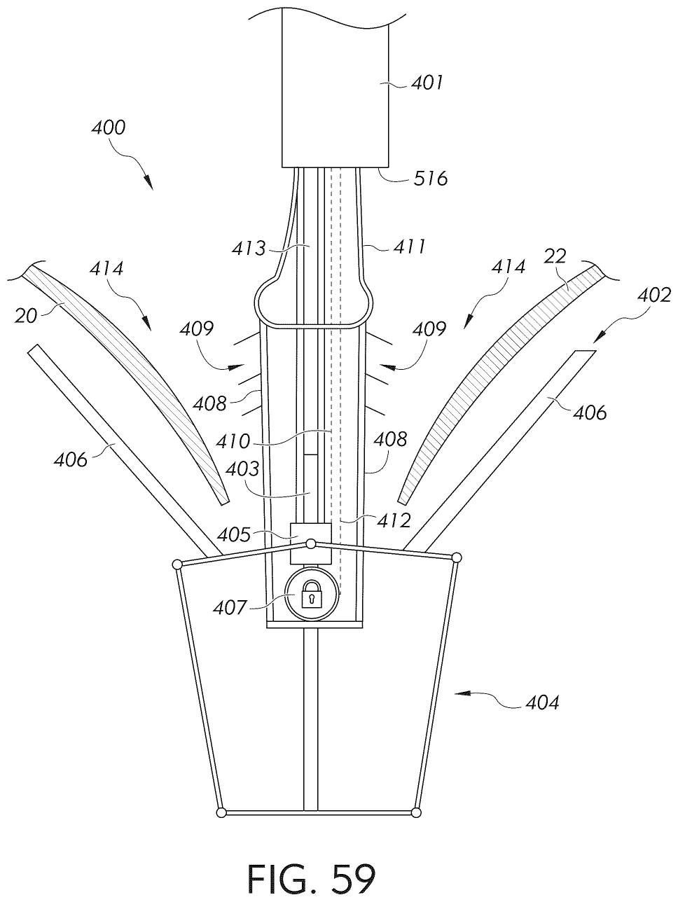

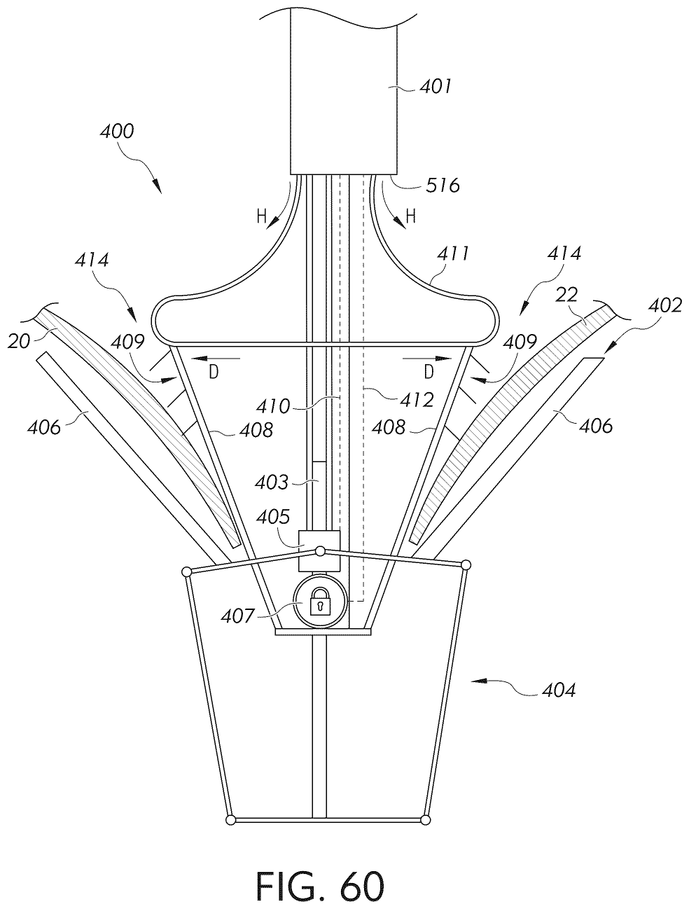

Referring to FIG. 59, the valve repair device 402 is shown such that valve tissue 20, 22 is disposed in the opening 414 between the gripping members 408 and the paddles 406. Referring to FIG. 60, after the valve tissue 20, 22 is disposed between the gripping members 408 and the paddles 406, the gripper control mechanism 411 is used to lessen the width of the opening 414 between the gripping members and the paddles. That is, in the illustrated example, the line of the gripper control mechanism 411 is released from or pushed out of the opening 516 of the delivery member in the direction H, which allows the gripping members 408 to move in the direction D to lessen the width of the opening 414. While the gripper control mechanism 411 is shown moving the gripping members 408 to increase the width of the opening 414 between the gripping members and the paddles 406 (FIG. 59), it should be understood that the gripping members may not need to be moved in order to position valve tissue in the opening 414. In certain circumstances, however, the opening 414 between the paddles 406 and the gripping members 408 can be wider in order to receive the valve tissue.

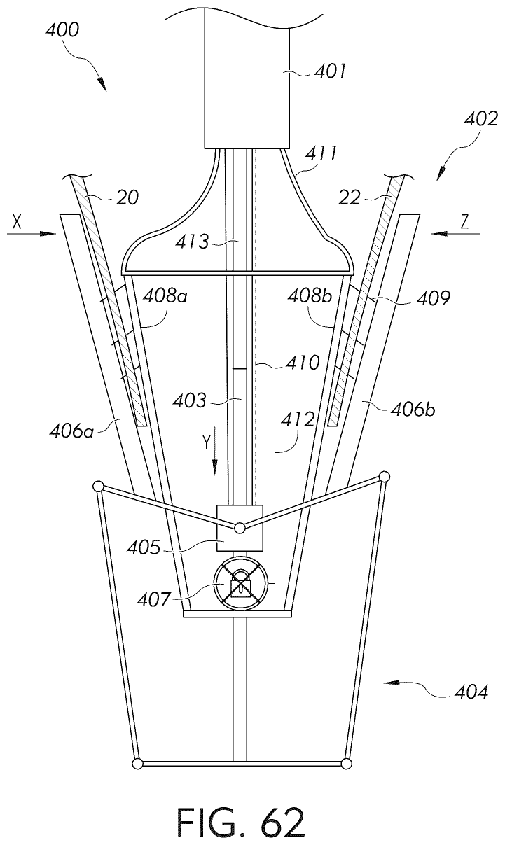

Referring to FIG. 62, the valve repair device 402 is in the closed position and secured to valve tissue 20, 22. The valve repair device 402 is secured to the valve tissue 20 by the paddles 406a, 406b and the gripping members 408a, 408b. In particular, the valve tissue 20,22 is attached to the valve repair device 402 by the gripping portion 409 of the gripping members 408a, 408b, and the paddles 406a, 406b engage the gripping members 408 to secure the valve repair device 402 to the valve tissue 20, 22.

In order to move the valve repair device 402 from the open position to the closed position, the lock 407 is moved to an unlocked condition (as shown in FIG. 62) by the lock control mechanism 412. Once the lock 407 is in the unlocked condition, the coupler 405 can be moved along the shaft 403 by the paddle control mechanism 410. In the illustrated example, the paddle control mechanism 410 moves the coupler 405 in a direction Y along the shaft, which causes one paddle 406a to move in a direction X and the other paddle 406b to move in a direction Z. The movement of the paddles 406a, 406b in the direction X and the direction Z, causes the paddles to engage the gripping members 408a, 408b and secure the valve repair device 402 to the valve tissue 20, 22.

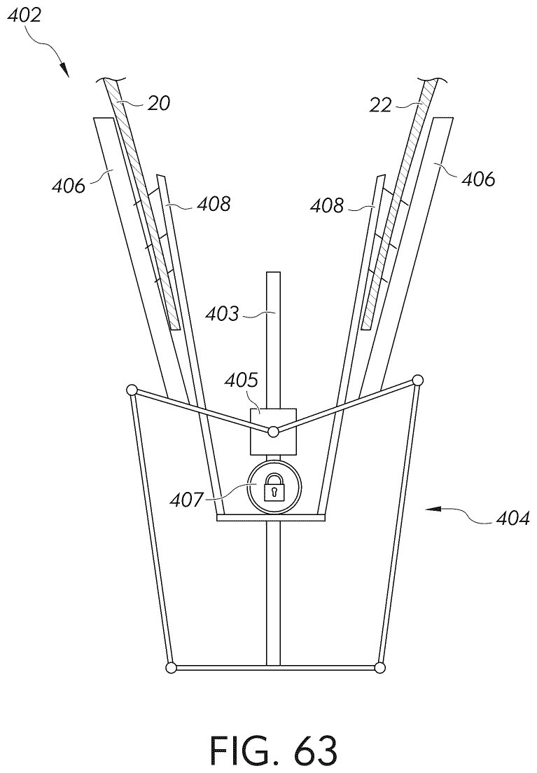

Referring to FIG. 63, after the paddles 406 are moved to the closed position to secure the valve repair device 402 to the valve tissue 20, 22 (as shown in FIG. 62), the lock 407 is moved to the locked condition by the lock control mechanism 412 (FIG. 62) to maintain the valve repair device 402 in the closed position. After the valve repair device 402 is maintained in the locked condition by the lock 407, the valve repair device 402 is removed from the delivery device 401 by disconnecting the shaft 403 from the placement shaft 413 (FIG. 62). In addition, the valve repair device 402 is disengaged from the paddle control mechanism 410 (FIG. 62), the gripper control mechanism 411 (FIG. 62), and the lock control mechanism 412. Removal of the valve repair device 402 from the delivery device 401 allows the valve repair device to remain secured to valve tissue 20, 22 while the delivery device 401 is removed from a patient.

During delivery and/or implantation of a device or implant in the native heart valve, movement of the device to the implanted position may be impeded or obstructed by the native heart structures. For example, articulable portions of a device or implant (such as paddle portions of anchors used to secure the device to the native heart valve tissue) may rub against, become temporarily caught, or be temporarily blocked by the chordae tendineae CT (shown in FIGS. 3 and 4) that extend to the valve leaflets. An example device or implant can be configured to reduce the likelihood of the device or implant getting temporarily caught or blocked by the CT. For example, the device or implant can take a wide variety of different configurations that are configured to be actively or passively narrowed to reduce the width of a paddle frame of an anchor portion of the device and, consequently, reduce the surface area of the device, which will make it easier to move the device/implant past and/or through the CT.

The valve repair device or treatment device 402 can include any other features described with respect to other devices discussed in the present application, and the device 402 can be positioned to engage valve tissue as part of any suitable valve repair system or treatment system (e.g., any valve repair or treatment system disclosed in the present application). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

In various implementations, a delivery apparatus is configured to make it easier to move the device or implant between its various configurations and/or to implant (and/or deliver) the device or implant in the native heart valve. For example, controls on a handle used in a delivery assembly can be configured to enable improved control of the device or implant, as will be described.

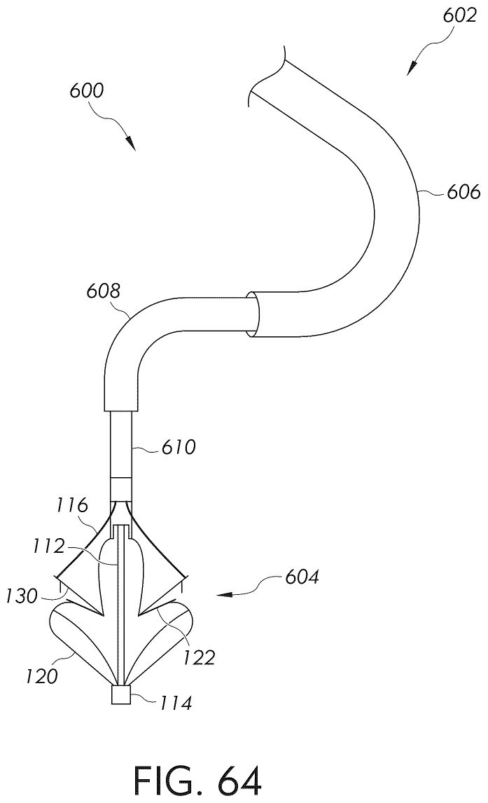

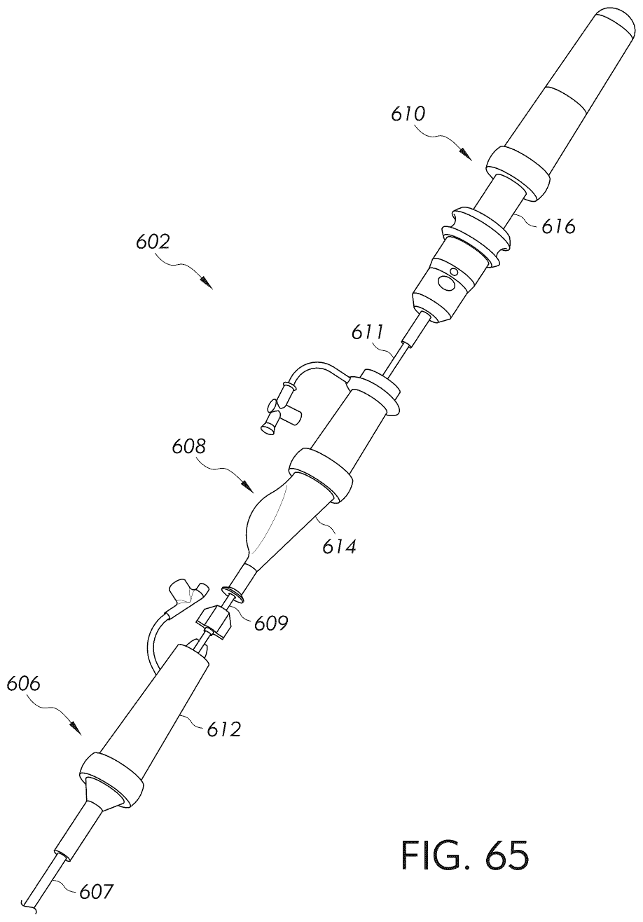

FIGS. 64-65 show an example of a delivery assembly 600 and its components. Referring to FIG. 64, the delivery assembly 600 can comprise the delivery apparatus 602 and a device 604 (e.g., a treatment device, a repair device, an implant, an implantable device, a valve repair device, etc.). The delivery apparatus 602 can comprise a plurality of catheter assemblies and optional catheter stabilizers (not shown in FIGS. 64 and 65). For example, in the illustrated example, the delivery apparatus 602 includes a delivery catheter assembly 606, a steerable catheter assembly 608, and a device/implant catheter assembly 610 (e.g., a control catheter assembly, a device control catheter assembly, an implant control catheter assembly, an implant delivery catheter assembly, etc.). The steerable catheter assembly 608 extends coaxially through the delivery catheter assembly 606, and the device/implant catheter assembly 610 extends coaxially through the steerable catheter assembly 608 and the delivery catheter assembly 606. The device 604 can be releasably coupled to a distal portion of the device/implant catheter assembly 610, as further described below. It should be appreciated that the device 604 can be any device described herein.

As shown in FIG. 65, each of the delivery catheter assembly 606, the steerable catheter assembly 608, and the device/implant catheter assembly 610 includes a sheath or shaft 607, 609, 611 extending from a handle 612, 614, 616, respectively. The handles 612, 614, 616 are located at a proximal end of each of the corresponding sheaths or shafts, and include one or more control members to enable a user to manipulate the catheter assembly (e.g., bend or rotate the sheath or shaft of the catheter assembly) or control a component coupled to the corresponding catheter assembly (e.g., a control wire extending through the shaft of the catheter assembly).

The delivery catheter assembly 606 and the steerable catheter assembly 608 can be used, for example, to access an implantation location (e.g., a native mitral valve region of a heart) and/or to position the device/implant catheter assembly 610 at the implantation location. Accordingly, in various implementations, the delivery catheter assembly 606 and the steerable catheter assembly 608 are configured to be steerable. Features of catheter assemblies disclosed by U.S. Pat. Nos. 10,653,862 and 10,646,342 can be used in the catheter assemblies 606, 608, 610. U.S. Pat. Nos. 10,653,862 and 10,646,342 are hereby incorporated by reference in their entireties.

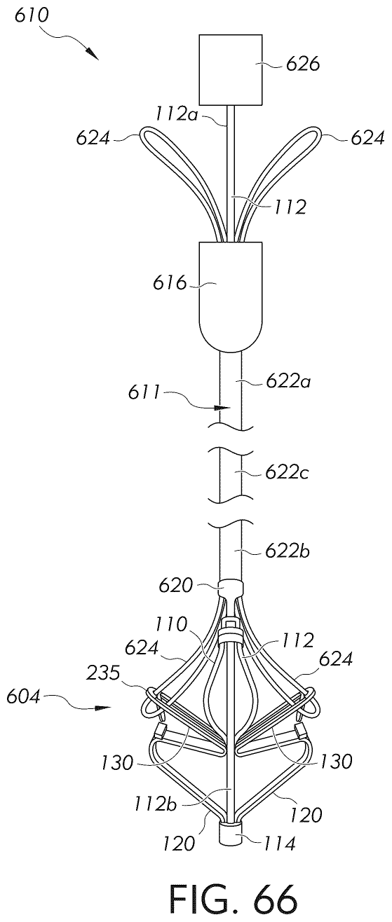

FIG. 66 illustrates an example of a device/implant catheter assembly 610. FIG. 66 illustrates a generalized device/implant catheter assembly 610. In the example illustrated by FIG. 66, the device/implant catheter assembly 610 can comprise the inner or actuation element 112, a coupler 620, an outer shaft 611, a handle 616 (shown schematically), and clasp actuation lines 624. A proximal end portion 622a of the outer shaft 611 can be coupled to extend distally from the handle 616, and a distal end portion 622b of the outer shaft 611 can be coupled to the coupler 620. The actuation element 112 can extend distally from the actuation control or knob 626 (shown schematically in FIG. 66), through the handle 616, through the outer shaft 611, and through the coupler 620. The actuation element 112 can be movable (e.g., axially and/or rotationally) relative to the outer shaft 611 and the handle 616. The clasp actuation lines 624 can extend through and be axially movable relative to the handle 616 and the outer shaft 611. The clasp actuation lines 624 can also be axially movable relative to the actuation element 112.

In some implementations, the outer shaft 611 of the device/implant catheter assembly 610 can be configured to be steerable. For example, although not shown, the device/implant catheter assembly 610 can comprise a pull element, such as a wire, and a flexible sleeve, such as a flexible axially non-compressible pull wire sleeve (e.g., a helical coil).

As shown in FIG. 66, the actuation element 112 (e.g., actuation rod, actuation tube, actuation shaft, actuation wire, etc.) of the device/implant catheter assembly 610 can be releasably coupled to the cap 114 of the device 604. The actuation element extends from a proximal end portion 112a to a distal end portion 112b. In some implementations, the distal end portion 112b of the actuation element 112 can comprise external threads configured to releasably engage interior threads of the cap 114 of the device 604. As such, rotating the actuation element 112 in a first direction (e.g., clockwise) relative to the cap 114 of the device 604 releasably secures the actuation element 112 to the cap 114, while rotating the actuation element 112 in a second direction (e.g., counterclockwise) relative to the cap 114 of the device 604 releases the actuation element 112 from the cap 114.

In the example of FIG. 66, the outer shaft 611 of the device/implant catheter assembly 610 is an elongate shaft extending axially between the proximal end portion 622a, which is coupled to the handle 616, and the distal end portion 622b, which is coupled to the coupler 620. The outer shaft 611 can also include an intermediate portion 622c disposed between the proximal and distal end portions 622a, 622b. The outer shaft 611 can be formed from various materials, including metals and polymers. For example, in one particular implementation, the proximal end portion 622a can comprise stainless steel and the distal and intermediate portions 622b, 622c can comprise polyether block amide (PEBA). The outer shaft 611 can also comprise an outer covering or coating, such as a polymer that is reflowed over the portions 622a, 622b, and 622c.

As shown in FIG. 66, the clasp actuation lines 624 are coupled to the clasps 130 through holes 235 in the clasps 130 and extend axially through the outer shaft 611 between the clasps 130 and the handle 616. In some implementations, the clasp actuation lines 624 are each coupled to a clasp control member 628 at the proximal end of the clasp actuation lines 624. Each clasp control member 628 can be, for example, an axially-moving control or slider coupled to a corresponding clasp actuation line 624 to axially move the clasp actuation line 624 relative to the outer shaft 611 and the actuation element 112. Each of the clasp control members 628 can be operated independently of the other clasp control member such that each clasp actuation line 624 is moved relative to the outer shaft 611, the actuation element 112, and the other clasp actuation line 624, or the clasp control members 628 can be fixed with respect to one another (e.g., locked) such that the clasp actuation lines 624 are axially moved together relative to the outer shaft 611 and the actuation element 112. Additional information on example clasp control members 628 and device/implant catheter assemblies including the same can be found, for example, in US Provisional Application Ser. No. 63/181,120, filed on Apr. 28, 2021, which is incorporated herein by reference in its entirety.

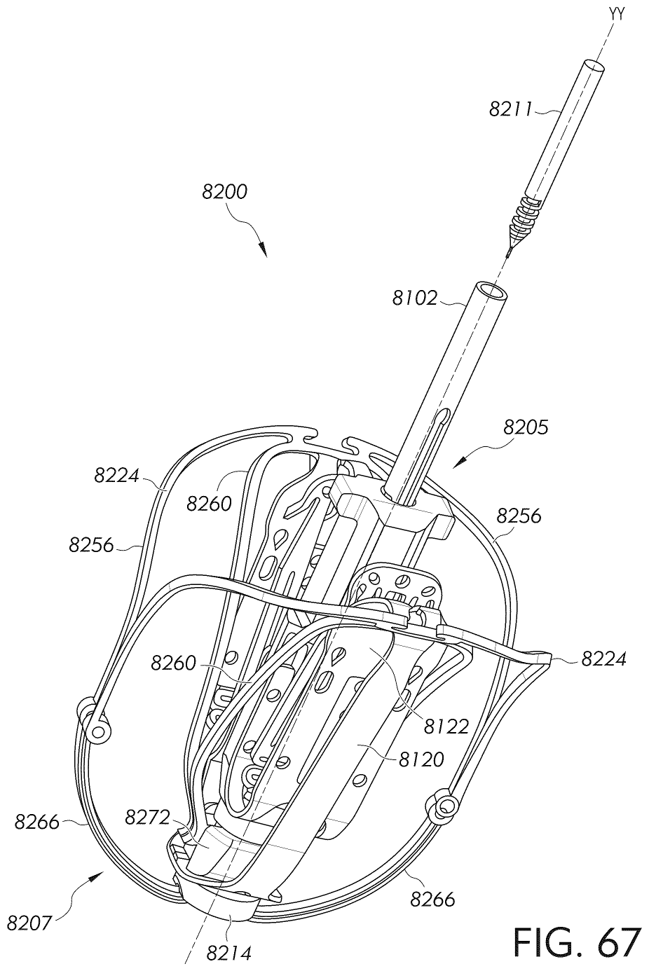

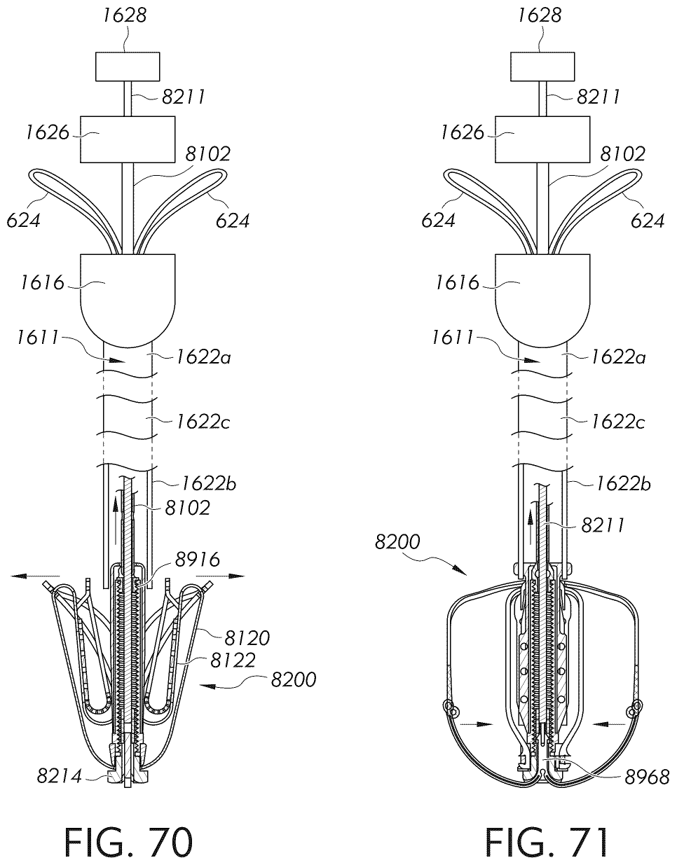

In some implementations, the catheter assembly is used to deliver and/or operate a device, such as the device 8200 illustrated in FIG. 67 or other treatment device or repair devices herein. FIGS. 67-71 illustrate one of the many valve repair systems or treatment systems for repairing or treating a native valve of a patient that the concepts of the present application can be applied to. Referring to FIGS. 70 and 71, the valve repair system or treatment system includes a delivery device 1611 and a device 8200, e.g., a treatment device, a repair device, a valve repair device, an implantable valve repair device, an implant, etc. Referring to FIGS. 67-69, the device 8200 includes a proximal or attachment portion 8205, paddle frames 8224, outer paddle portions 8120, inner paddle portions 8122, and a distal portion 8207. The proximal portion 8205, the distal portion 8207, and the paddle frames 8224 can be configured in a variety of ways. The outer paddle portions 8120, inner paddle portions 8122, and paddle frames 8224 can open and close in the same or similar manner as the outer paddle portions 220, inner paddle portions 222, and paddle frames 224 described above.

In the example illustrated in FIG. 67, the paddle frames 8224 can be symmetric along longitudinal axis YY. However, in some implementations, the paddle frames 8224 are not symmetric about the axis YY. Moreover, referring to FIG. 67, the paddle frames 8224 include outer frame members 8256 and inner frame members 8260.

In some implementations, a connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 69). Between the connector 8266 and the proximal portion 8205, the outer frame portions 8256 form a curved shape. For example, in the illustrated example, the shape of the outer frame portions 8256 resembles an apple shape in which the outer frame portions 8256 are wider toward the proximal portion 8205 and narrower toward the distal portion 8207. In some implementations, however, the outer frame portions 8256 can be otherwise shaped.

The inner frame members 8260 extend from the proximal portion 8205 toward the distal portion 8207. The inner frame members 8260 then extend inward to form retaining portions 8272 that are attached to the actuation cap 8214. The retaining portions 8272 and the actuation cap 8214 can be configured to attach in any suitable manner.

In some implementations, the inner frame members 8260 are rigid frame portions, while the outer frame members 8256 are flexible frame portions. The proximal end portion of the outer frame members 8256 connects to the proximal end portion of the inner frame members 8260, as illustrated in FIG. 67.

The width adjustment element 8211 (e.g., width adjustment wire, width adjustment shaft, width adjustment tube, width adjustment line, width adjustment cord, width adjustment suture, width adjustment screw or bolt etc.) is configured to move the outer frame members 8256 from the expanded position to the narrowed position by pulling the inner end 8968 (FIG. 69) and portions of the connector 8266 into the actuation cap 8214. The actuation element 8102 (e.g., as a wire, rod, shaft, tube, screw, suture, line, strip, combination of these, etc.) is configured to move the inner paddle frame portions 8260 to open and close the paddles in accordance with some implementations disclosed herein.

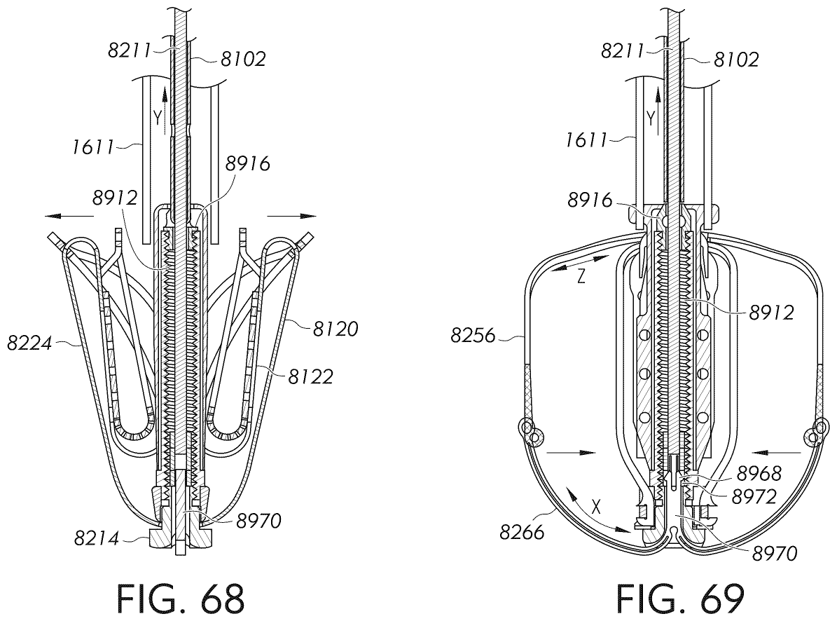

As shown in FIGS. 68 and 69, the paddle frames 8224 have an inner end 8968 that engages with the width adjustment element 8211 such that a user can move the inner end 8968 relatively inside the receiver 8912 (e.g., an internally threaded element, a column, a conduit, a hollow member, a notched receiving portion, a tube, a shaft, a sleeve, a post, a housing, tracks, a cylinder. etc.) to move the outer frame members 8256 between a narrowed position and an expanded position. In the illustrated example, the inner end 8968 includes a post 8970 that attaches to the outer frame members 8256 and a coupler 8972 that extends from the post 8970. The coupler 8972 is configured to attach and detach from both the width adjustment element 8211 and the receiver 8912. When the coupler 8972 is attached to the width adjustment element 8211, the coupler is released from the receiver 8912. When the coupler 8972 is detached from the width adjustment element 8211, the coupler is secured to the tube. The inner end 8968 can, however, be configured in a variety of ways. Any configuration that can suitably attach the outer frame members 8256 to the coupler to allow the width adjustment element 8211 to move the outer frame members 8256 between the narrowed position and the expanded position can be used.

The width adjustment element 8211 allows a user to expand or contract the outer frame members 8256 of the device 8200. In the example illustrated in FIGS. 68 and 69, the width adjustment element 8211 includes an externally threaded end that is threaded into the coupler 8972. The width adjustment element 8211 moves the coupler in the receiver 8912 to adjust the width of the outer frame members 8256. When width adjustment element 8211 is unscrewed from the coupler 8972, the coupler engages the inner surface of the receiver 8912 to set the width of the outer frame members 8256.

In some implementations, the receiver 8912 can be integrally formed with or fixedly connected to a distal cap 8214. Moving the cap 8214 relative to a body of the attachment portion 8205 opens and closes the paddles. In the illustrated example, the receiver 8912 slides inside the body of the attachment portion 8205. When the coupler 8972 is detached from the width adjustment element 8211, the width of the outer frame members 8256 is fixed while the actuation element 8102 moves the receiver 8912 and cap 8214 relative to a body of the attachment portion 8205. Movement of the cap can open and close the device in the same manner as the other implementations disclosed above.

In the illustrated example, a driver head 8916 is disposed at a proximal end of the actuation element 8102. The driver head 8916 releasably couples the opening/closing control actuation element 8102 to the receiver 8912. In the illustrated example, the width adjustment element 8211 extends through the actuation element 8102. The actuation tube is axially advanced in the direction opposite to direction Y to move the distal cap 8214. Movement of the distal cap 8214 relative to the attachment portion 8205 is effective to open and close the paddles, as indicated by the arrows in FIG. 68. That is movement of the distal cap 8214 in the direction Y closes the device and movement of the distal cap in the direction opposite to direction Y opens the device.

Also illustrated in FIGS. 68 and 69, the width adjustment element 8211 extends through the actuation element 8102, the driver head 8916, and the receiver 8912 to engage the coupler 8972 attached to the inner end 8968.

The movement of the outer frame members 8256 to the narrowed position can allow the device or implant 8200 to maneuver more easily into position for implantation in the heart by reducing the contact and/or friction between the native structures of the heart—e.g., chordae—and the device 8200. The movement of the outer frame members 8256 to the expanded position provides the anchor portion of the device or implant 8200 with a larger surface area to engage and capture leaflet(s) of a native heart valve.

Referring to FIGS. 70 and 71, an implementation of a device/implant catheter assembly 1611 (e.g., a control catheter assembly, a device control catheter assembly, an implant control catheter assembly, an implant delivery catheter assembly, etc.) in which clasp actuation lines 624 extend through a handle 1616, the actuation element 8102 is coupled to an actuation control 1626 (which may be referred to herein as an anchor actuation control or paddle actuation control, etc.), and the width adjustment element 8211 is coupled to a width control 1628 (which may be referred to herein as an anchor width control, paddle width control, etc.). A proximal end portion 1622a of the shaft or catheter of the device/implant catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the shaft or catheter of the delivery device 1611, and through the proximal end of the device 8200, where it couples with the driver head 8916. The actuation element 8102 can be axially movable relative to the shaft of the device/implant catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft 1611, and through the device 8200), where it couples with the movable coupler 8972. The width adjustment element 8211 can be axially movable relative to the actuation element 8102, the outer shaft 1611, and the handle 1616. The clasp actuation lines 624 can extend through and be axially movable relative to the handle 1616 and the outer shaft 1611. The clasp actuation lines 624 can also be axially movable relative to the actuation element 8102.

Referring to FIGS. 70 and 71, the width adjustment element 8211 can be releasably coupled to the coupler 8972 of the device 8200. Advancing and retracting the width adjustment element 8211 with the control 1628 widens and narrows the paddles. Advancing and retracting the actuation element 8102 with the control 1626 opens and closes the paddles of the device.

In the examples of FIGS. 70 and 71, the catheter or shaft of the device/implant catheter assembly 1611 is an elongate shaft extending axially between the proximal end portion 1622a, which is coupled to the handle 1616, and the distal end portion 1622b, which is coupled to the device 8200. The outer shaft 1611 can also include an intermediate portion 1622c disposed between the proximal and distal end portions 1622a, 1622b.

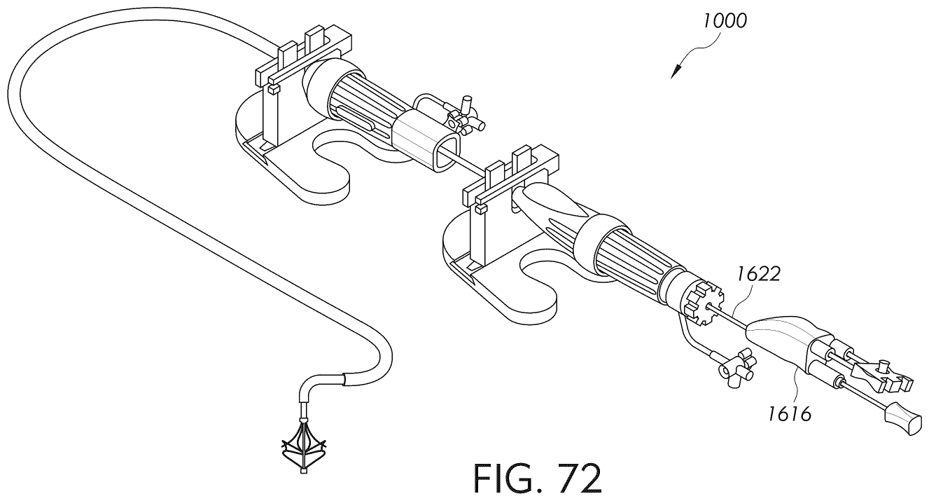

In various implementations, the handle 1616 including the controls for adjusting the width of the paddle frames can be incorporated into a control system having a variety of forms. For example, the handle 1616 can be incorporated into the control system 1000 shown in FIG. 72. Features of the control system illustrated in FIG. 72 can be found in U.S. Pat. No. 10,820,998, filed Apr. 8, 2020, and entitled “Valve Repair Device,” the entire contents of which is incorporated by reference herein.

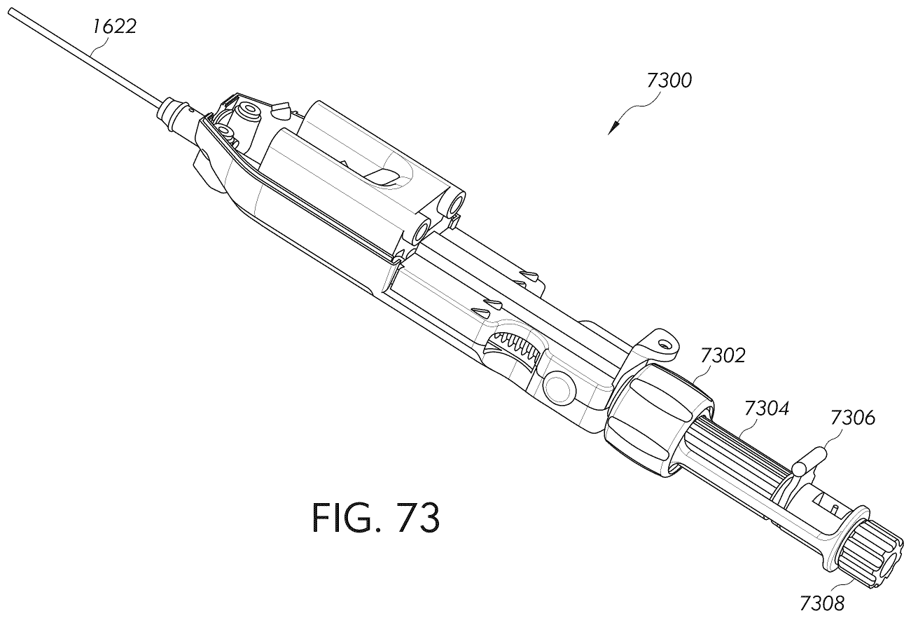

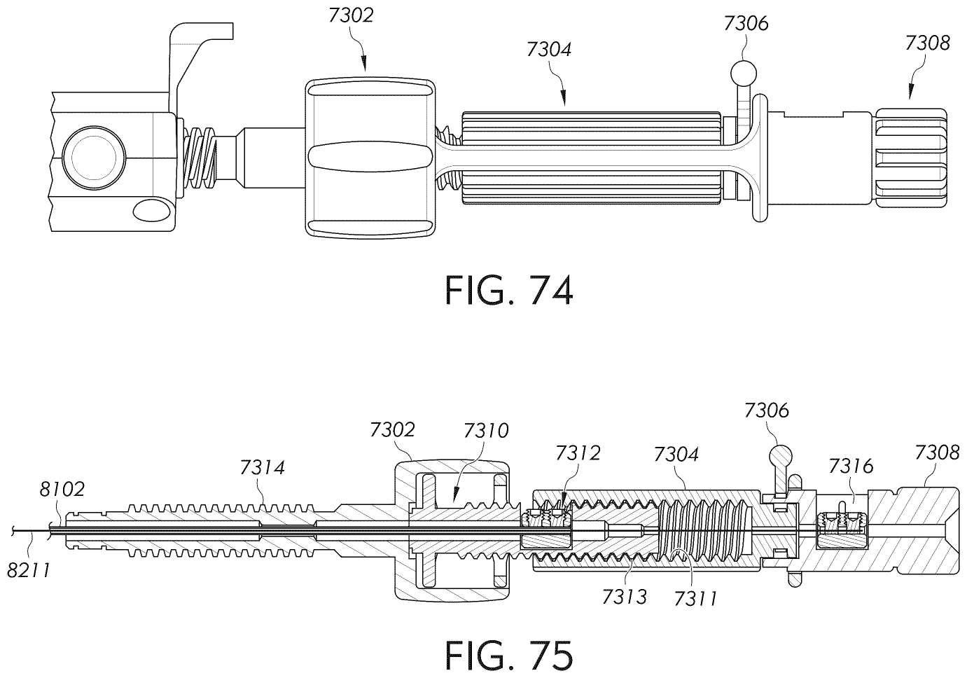

FIGS. 73-75 depict an example implementation of a handle 7300 incorporating paddle width adjustment controls. The handle 7300 can be used to position the device/implant catheter 1622 to position the device, advance and retract the actuation element 8102 to open and close the device, and advance and retract the width adjustment element 8211 to widen and narrow the paddle frames (see FIG. 75). As shown in FIG. 73, the handle 7300 generally includes a paddle actuation control 7302, a paddle width control 7304, a paddle release clip 7306, and a width control element connection control 7308. FIG. 74 shows the paddle actuation control 7302, paddle width control 7304, paddle release clip 7306, and the control element connection control 7308 in greater detail. FIG. 75 shows a cross-section of the paddle actuation control 7302, paddle width control 7304, paddle release clip 7306, and the control element connection control 7308.

It should be appreciated that handle 7300 can be an implementation of the handle 1616 in FIGS. 70 and 71. For example, the paddle actuation control or knob 7302 is an example of the paddle actuation control 1626 in FIGS. 70 and 71, and the paddle width control or knob 7304 is an example of the paddle width control 1628 in FIGS. 70 and 71. Different types of controls/controllers can be used for any of the controls or knobs described herein, e.g., knobs as depicted in many of the figures or other controls such as buttons, switches, gears, etc. can be used. Below the term “knob” can be used to match the variation depicted in the figures, but it should be understood that this is an example and other types of controls or controllers could be used instead of knobs.

The paddle actuation knob 7302 is rotatable about axis A-A extending longitudinally along the handle 7300. The actuation element 8102 extends through the paddle actuation knob 7302 and is fixed to a ferrule 7312. The ferrule 7312 is affixed to a frame 7310 that includes external threads for engaging with internal threads of the paddle width control knob 7304, as will be described in greater detail below. The distal end of the paddle actuation knob 7302 includes external threads 7314, which are configured to engage internal threads in a housing of the handle 7300. The paddle actuation knob 7302 is rotatable relative to both the housing of the handle 7300 and relative to the frame 7310 and attached ferrule 7312. Accordingly, as the paddle actuation knob 7302 is rotated about the axis A-A with respect to the handle 7300, the paddle actuation knob 7302 axially drives the frame 7310 and attached ferrule 7312 (and, therefore, the actuation element 8102) with respect to the handle 7300, which is effective to axially drive the actuation element 8102 with respect to the device 8200.

The paddle width control knob 7304 is also rotatable about axis A-A extending longitudinally along the handle 7300. The width adjustment element 8211 extends through the actuation element 8102, the paddle actuation knob 7302, the ferrule 7312, and the paddle width control knob 7304. The width adjustment element 8211 is fixedly attached to a ferrule 7316 that is connected inside the control element connection knob 7308. The ferrule 7316 fixes the width adjustment element 8211 rotationally and axial with respect to the control element connection knob 7308. The paddle width control knob 7304 is rotatably coupled to the control element connection knob 7308 and relative to the width adjustment element 8211.

In some implementations, the paddle width control knob 7304 is threadably connected to a threaded component that is fixed or part of the paddle actuation assembly. When the paddle width control knob 7304 is rotated, the threaded connection drives or forces the paddle width control knob 7304 and coupled with control element 7316, such as the illustrated ferrule, away from the paddle actuation knob 7302 and coupled actuation element 7312 or ferrule. For example, as shown in the FIG. 75 implementation, the width control knob 7304 (e.g., paddle width control knob, etc.) includes internal threads 7311 that engage with the external threads 7313 of the frame 7310. Accordingly, rotation of the paddle width control knob 7304 is effective to axially move the paddle width control knob 7304. Since the paddle width control knob 7304 is rotatably coupled to the control element connection knob 7308, the rotation of the paddle width control knob 7304 axially, but not rotatably, moves the width adjustment element 8211 with respect to the paddle actuation knob 7302. This is effective to axially drive the width adjustment element 8211 with respect to the attachment portion 8205 of the device 8200. Movement of the width adjustment element 8211 with respect to the attachment portion 8205 is effective to adjust the width of the paddles, as described hereinabove.

The control element connection knob 7308 is coupled to a proximal end of the paddle width control knob 7304 and includes the ferrule 7316 to which the width adjustment element 8211 is affixed. In implementations, the control element connection knob 7308 does not rotate with respect to the axis A-A during implantation of the device 8200. The paddle release clip 7306 locks the control element connection knob 7308 and the paddle width control knob 7304 axially together, while allowing rotation there between. Accordingly, to disengage the width adjustment element 8211 from the device 8200, the paddle release clip 7306 is removed and the control element connection knob 7308 is rotated with respect to the paddle width control knob 7304. This rotates the width adjustment element 8211 with respect to the device 8200, which is effective to uncouple the width adjustment element 8211 from the device 8200. The control element connection knob 7308 and the width adjustment element 8211 can then be pulled axially to remove the width adjustment element from the device 8200.

In some implementations, the device 8200 is released from the device/implant catheter 1622 by the following sequence of actions by the handle. The paddle release clip 7306 is removed and the control element connection knob 7308 is rotated to uncouple the width adjustment element 8211 from the device 8200. The control element connection knob 7308 and the width adjustment element 8211 can then be pulled proximally past a distal end of the actuation element 8102 to decouple the actuation element 8102 driver head 8916 and connected receiver, such as the illustrated tube. For example, in one implementation fingers at the distal end flex inward when the width adjustment element 8211 is pulled proximally past the distal end of the actuation element 8102 to decouple the actuation element 8102 from the driver head 8916. The paddle width control knob 7304 and the actuation element 8102 can then be pulled proximally past a distal end or coupler of the catheter 1622 to decouple the distal end or coupler of the catheter 1622 from the device 8200. For example, in one implementation fingers at the distal end or coupler flex outward when the actuation element 8102 is pulled proximally past the distal end or coupler of the catheter 1622 to decouple the catheter 1622 from the device 8200.

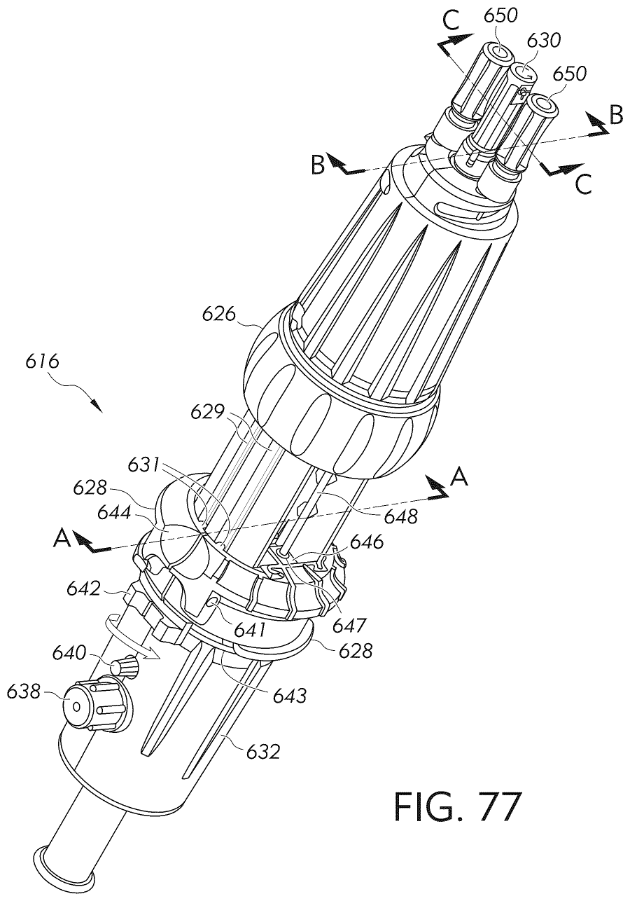

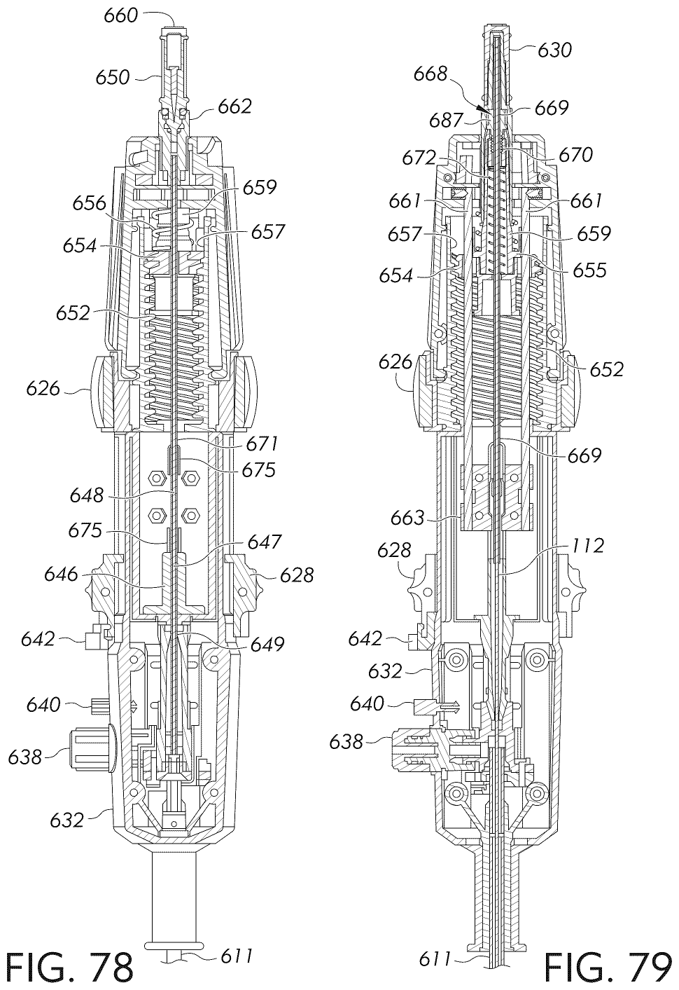

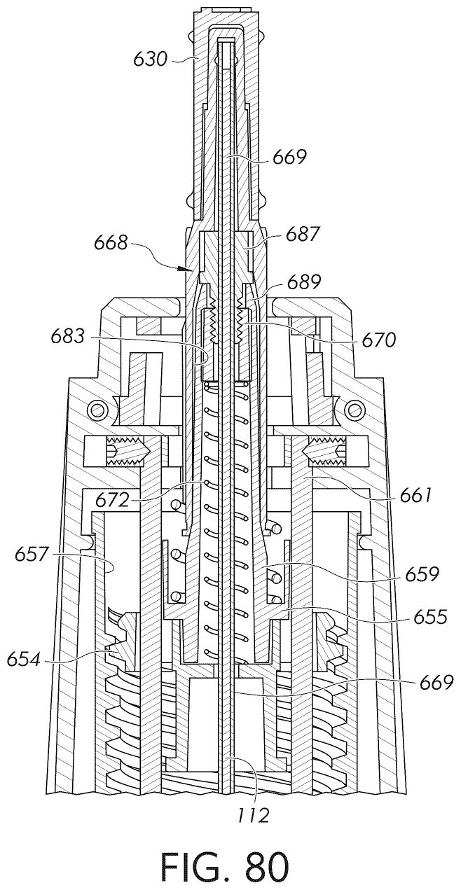

Turning now to FIG. 77, an implementation of a handle 616 of a device/implant catheter assembly 610 (e.g., a control catheter assembly, a device control catheter assembly, an implant control catheter assembly, an implant delivery catheter assembly, etc.) is shown. In FIG. 77, the handle 616 includes a housing 632 to which the various controls are coupled. The housing 632 can be formed from various materials, including polymers such as polycarbonate, and can be formed as a unitary body (e.g., through injection molding) or fastened together in any one of a variety of manners, including fasteners, pins, adhesives, or the like. As shown in FIGS. 78-81, the housing 632 comprises a plurality of lumens, through which the clasp actuation lines 624 and the actuation element (e.g., actuation rod, actuation tube, actuation wire, actuation shaft, etc.) 112 extend.

The handle 616 further comprises a flush port 638, as shown in FIGS. 77-79. The flush port 638 is configured for flushing (e.g., with a saline solution) the outer shaft 611 prior to inserting the outer shaft 611 into a patient's vasculature. Additionally, the flush port 638 enables air present in the outer shaft 611 to be removed as fluid is drawn into the proximal end of the outer shaft 611. Additional information regarding a flush port that can be used in the handle 616 can be found in, for example, International Publication No. WO 2020/112622, the entire contents of which is incorporated by reference herein.

As shown in FIGS. 77-79, the housing 632 comprises an aperture for receiving a marking pin 640. The marking pin 640 can be removably coupled with the aperture of the housing 632 for storage of the marking pin 640. During use, the marking pin 640 can be removed from the aperture of the housing and inserted into an aperture 641 (shown in FIG. 77) on one of the clasp control members 628. For example, the marking pin 640 can be inserted into an aperture 641 on the clasp control member 628 that controls the clasp to be coupled to a posterior leaflet of the mitral valve. Accordingly, in the event that the handle 616 is rotated during use, the user can readily identify the clasp control member 628 for control of the clasp to be coupled to the posterior leaflet (e.g., the marked clasp control member 628) and the clasp control member 628 for control of the clasp to be coupled to the anterior leaflet (e.g., the unmarked clasp control member 628). Although the marking pin 640 is described as being coupled to the clasp control member 628 that controls the clasp to be coupled to the posterior leaflet, it is contemplated that the marking pin 640 can be coupled to either of the clasp control members 628 to indicate any particular orientation of the device/implant catheter assembly.

Also included on the handle 616 is a slide lock 642 that is configured to slide between a first position, in which the slide lock 642 is coupled to one of the clasp control members 628, to a second position, in which the slide lock 642 is coupled to both clasp control members 628. For example, each of the clasp control members 628 can include a flange 643 to which the slide lock 642 is slidably coupled. Accordingly, the slide lock 642 can be moved to the first position (as indicated by the arrow shown in FIG. 77) in which the slide lock is engaged with the flange 643 of one of the clasp control members 628 (e.g., to enable independent movement of each clasp actuation line relative to the other) and the second position (shown in the FIG. 77) in which the slide lock 642 is engaged with the flange 643 of both of the clasp control members 628 (e.g., to enable simultaneous movement of the clasp actuation lines) in order to selectively control relative movement between the clasp control members 628. When the slide lock is in the first position, each of the clasp control members is axially movable independently of the other clasp control member, and when the slide lock is in the second position, the clasp control members are axially movable together. Although not shown in the figures, in implementations, each flange 643 can include a stop to limit the position of the slide lock 642 along the flange 643.

In implementations, each of the clasp control members 628 can include one or more tactile or visual indicators to enable the user to differentiate one clasp control member from the other with improved accuracy. For example, one clasp control member 628 can have a different color and/or texture (e.g., ribbed, smooth) than the other clasp control member 628. As shown in the figures, in implementations, each of the clasp control members 628 is wrapped approximately 180 degrees around the circumference of the housing 632 such that together the clasp control members 628 surround and encircle the housing 632. Such an arrangement can, for example, enable the clasp control members 628 to be accessible to the user from any angle with a single hand. Moreover, in the implementation depicted in FIG. 68, the clasp control members 628 include a depressed region 644 that is generally shaped to receive and cradle the thumb of the user. Although the inclusion of such a depressed region is optional in some implementations, it can provide comfort for the user and improve the ease of use of the clasp control members 628 when it is included.

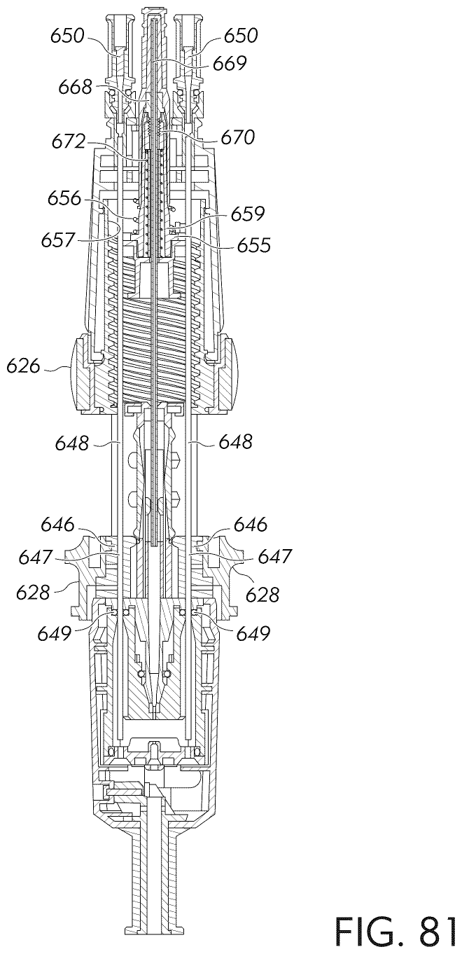

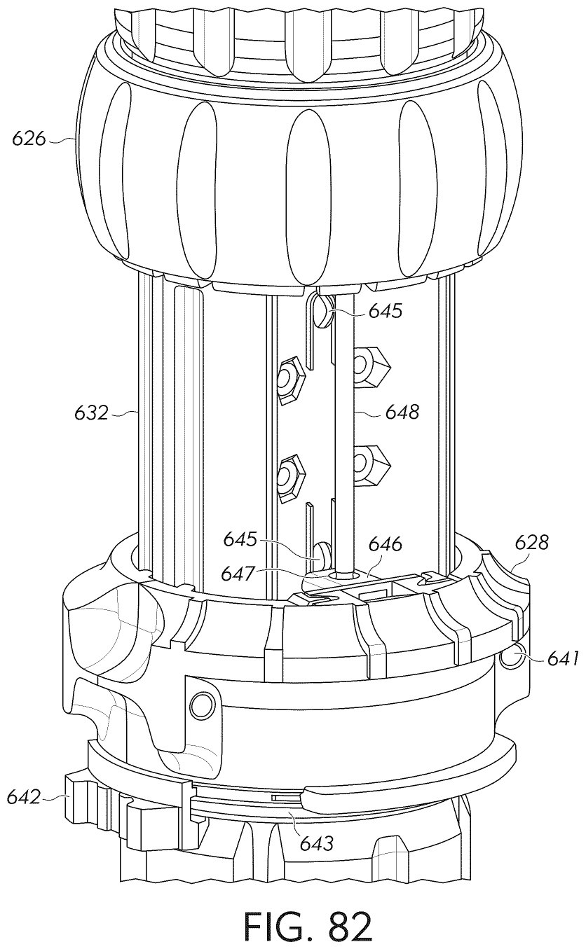

Each clasp control member 628 can further comprise one or more keyed projections or tongues that are configured to be received by and slide along a corresponding groove or slot in the housing 632. As best shown in FIG. 82, in implementations, the housing 632 further includes a pair of detents 645. A first detent 645 is located at a first axial position along a path of one of the clasp control members 628, and a second detent 645 is located at a second axial position along the path of the clasp control member 628. For example, in FIG. 82, one of the detents 645 projects from the housing 632 at a location that places the clasp control member 628 in a fully proximal position (e.g., a fully open position), thereby maintaining the clasp control member 628 in the fully proximal position (e.g., a fully open position) and preventing or inhibiting the clasp control member 628 from moving distally without intention of the user. A second one of the detents 645 projects from the housing 632 at a location that places the clasp control member 628 in a fully distal position (e.g., a fully closed position), thereby maintaining the clasp control member 628 in the fully distal position (e.g., a fully closed position) and preventing or inhibiting the clasp control member 628 from moving proximally without intention of the user. To release the clasp control member 628 for operation of the clasps, the user simply presses the clasp control member 628 with enough force to depresses the detent 645 and slide over the detent 645. When the clasp control member 628 moved to either the proximal position (open) or distal position (closed), the corresponding detent 645 provides tactile and audible feedback to the user indicative that the clasp control member 628 is in the open or closed position.

Referring to FIG. 82, the clasp control member 628 can comprise a coupler 646 configured to attach the clasp control member 628 to a clasp control tube 648. As described in more detail below, the clasp control tube 648 is releasably coupled to the clasp control lines 624. Sliding movement of the clasp control member 628 along the housing moves the clasp control tube 648 relative to the housing 632 to open and close the clasps.

In the example illustrated by FIGS. 77 and 78, the coupler 646 includes at least one aperture 647, in which the clasp control tube 648 is connected. The clasp control member 628 is slidably coupled to the housing 632, such as by the mating channels 629 of the housing 632 and the projections 631 of the clasp control member 628 (see FIG. 77) and/or by the clasp control tube 648 extending through a guide passage 649 formed in the housing (see FIG. 81). The clasp control member, the coupler 646, and the clasp control tube 648 can slide together axially (i.e., distally and proximally) with respect to the housing 632 to open and close the clasp. Only a single coupler 646 and clasp control tube 648 can be seen in FIGS. 77 and 78. FIG. 81 shows that each clasp control member 628 is fixedly coupled and slides in conjunction with a corresponding clasp control tube 648 in the same manner. The clasp control tube 648 can be fixed at a proximal end to a suture lock 650 that is used to secure the clasp control line 624 to the to the clasp control tube 648.

In some implementations, each clasp control tube 648 can comprise one or more optional keying features 671 at various positions or all along the axial length of the clasp control tube 648. In some implementations, no keying features are included. When included, the optional keying features 671 are complementary to corresponding keying features 675 formed in one or more components of the handle 616, such as in the housing 632 of the handle. These keying features 671, 675 can prevent or inhibit rotation of the clasp control tube 648 in the housing and thereby limit movement of the clasp control tube to linear movement along the length of the housing 632. For example, the optional keying feature 671 of the clasp control tube 648 can comprise a wire welded to the external surface of the clasp control tube 648 (see FIG. 78). In such implementations, one or more portions of the axial path defined by the handle for the clasp control tube 648, can include a corresponding groove configured to receive the wire and provide a path along which the wire can slide. The optional keying features 671, 675 can enhance stabilization of the clasp control member 628 by preventing or inhibiting rotation or torquing of the clasp control tube 648 within the handle 616. For example, the optional features 671, 675 can prevent or inhibit rotation or torquing of the clasp control tube 648 within the handle 616 when the control lines are pulled by the clasp control tube 648 to open the clasps and/or the control lines are pulled through the clasp control tube 648 to release the clasps.

The suture lock can take a wide variety of different forms. In the example illustrated by FIG. 88, the suture lock 650 comprises a post 658, a suture lock body 660, and a suture lock body receptacle 662. In the illustrated example, the suture lock body 660 has external threads 665 that mate with internal threads 667 of the suture lock body receptacle 662 to connect the suture lock body and the suture lock body receptacle together. The suture lock body 660 includes a central bore that extends from a first end to a second end of the suture lock body 660. In implementations, the central bore of the suture lock body 660 has a diameter that varies from the first end to the second end of the suture lock body 660. At least a portion of the central bore is sized to receive the post 658. Accordingly, the diameter of the central bore of the suture lock body 660 can vary to form a flange to limit the position of the post 658 within the bore of the suture lock body 660 while enabling the post 658 to be inserted into a first end of the suture lock body 660.

The suture lock body receptacle 662 includes a central bore that extends from a first end to a second end of the suture lock body receptacle 662. The central bore of the suture lock body receptacle 662 is sized to receive the suture lock body 660 at threaded end of the suture lock body receptacle 662 and is sized to receive and be attached to the clasp control tube 648 at the second end of the suture lock body receptacle 662. An optional O-ring 664 is positioned around the suture lock body 660 to form a fluid-tight seal between the suture lock body 660 and the suture lock body receptacle 662.

The clasp actuation line 624 is fixed at one end of the clasp actuation line 624 to the post 658, which is inserted into the suture lock body 660, thereby coupling the clasp actuation line 624 to the suture lock body 660. In implementations, the clasp actuation line 624 can be welded, adhered, or otherwise fixedly coupled to the post 658.

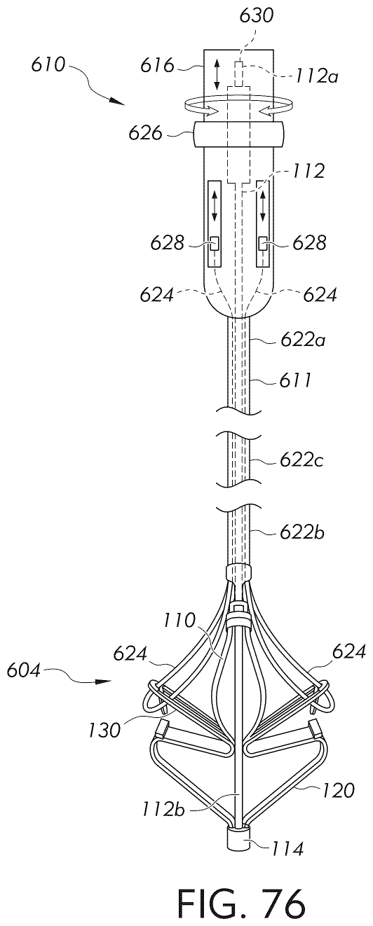

The clasp actuation line 624 is threaded through the central bore in the suture lock body 660, through the central bore in the suture lock body receptacle 662, and through the clasp control tube 648. The clasp control tube 648 guides and protects the clasp actuation line 624 through the interior of the handle 616. The clasp actuation line 624 exits the clasp control tube 648 near the distal end of the handle 616 and extends through the outer shaft 611 of the device/implant catheter assembly 610. As described herein, the clasp actuation line 624 exits the outer shaft 611 at the distal end of the outer shaft, and is coupled to the device, such as by passing through one or more holes 235 in the clasp 130 (see, e.g., FIGS. 66 and 76). The clasp actuation line 624 is then threaded back through the outer shaft 611 from the distal end to the proximal end and through the clasp control tube 648 to form a loop in the clasp actuation line 624 that extends from the distal end of the outer shaft 611. The clasp actuation line 624 then exits the clasp control tube 648 and passes between the suture lock body 660 and the suture lock body receptacle 662 to exit the central bore of the suture lock body receptacle 662. The threaded connection 665, 667 between the suture lock body 660 and the suture lock body receptacle 662 can be tightened to pinch the clasp actuation line 624 in position between a tapered nose 679 of the suture lock body 660 and a reduced diameter passage 681 of the suture lock body receptacle 662. Tightening the threaded connection 665, 667 also compresses the seal, such as an O-ring, between the suture lock body 660 and the suture lock body receptacle 662 to form a fluid-tight seal between the suture lock body 660 and the suture lock body receptacle 662.

To release the clasp from the clasp actuation line, the suture lock body 660 is removed (e.g., by unscrewing) from the suture lock body receptacle 662, freeing the end of the clasp actuation line 624 pinched between the suture lock body 660 and the suture lock body receptacle 662. Once the clasp actuation line 624 is released, the clasp actuation line can be pulled through the clasp control tube 648, the outer shaft 611, the hole 235 of the clasp and back through the outer shaft 611 and clasp control tube 648. As such, the clasp actuation line 624 is no longer connected to the clasp and is removed from the patient.

Returning to FIG. 78, as the clasp control member 628 is moved in a proximal direction, the clasp control tube 648 also moves in the proximal direction, pushing the suture lock 650 in the proximal direction with respect to the handle 616. As the suture lock 650 moves in the proximal direction, the loop of the clasp actuation line 624 extending from the distal end of the outer shaft 611 moves proximally, pulling the clasp 130. To release the clasp 130, such as to grasp the leaflet, the clasp control member 628 is moved in a distal direction, which in turn moves the clasp control tube 648 and suture lock 650 in the distal direction, thereby moving the loop of the clasp actuation line 624 in the distal direction, enabling the clasp 130 to move toward the inner paddles 122, grasping the leaflet between the clasp 130 and the inner paddle 122.

As previously mentioned, the handle 616 further comprises a knob 626 (or other control, e.g., button, switch, gears, etc.) that is configured to control the position of the actuation element 112 relative to the handle 616 and outer shaft 611. In some implementations, the knob 626 is configured to rotate about an axis of the handle 616. As can be seen in FIGS. 78-81, the knob 626 can be fixedly coupled to an internally threaded tube 652 positioned within the housing 632 of the handle 616.

In some implementations, when the knob 626 is rotated about the axis of the handle 616, the internally threaded tube 652 rotates with respect to the housing 632. An externally threaded nut or retractor 654 is positioned within and engaged with the internally threaded tube 652. The externally threaded nut or retractor 654 and is rotationally fixed with respect to the housing 632. The externally threaded nut or retractor 654 can be rotationally fixed in a wide variety of different ways. In the example illustrated by FIG. 79, a pair of guide rods 661 extend axially within the handle 616, and each of the pair of guide rods 661 is fixed to the housing 632 at both ends of the guide rod 661. A mounting bracket 663 couples the pair of guide rods 661 to the housing 632 at the distal end of each of the pair of guide rods 661. Accordingly, as the internally threaded tube 652 is rotated, the externally threaded retractor 654 is advanced linearly in an axial direction (i.e., distally and proximally) along the pair of guide rods 661.

The externally threaded retractor 654 is connected to the actuation element 112 such that linear movement of the externally threaded retractor 654 causes linear movement of the actuation element 112, thereby moving the device between a fully elongated configuration, an open configuration, and a closed configuration, as described herein. The translation of the rotational movement of the knob 626 into linear motion of the actuation element 112 can result in improved control and precision, thereby leading to improved precision during the opening and closing of the device.

The internally threaded tube 652 includes and unthreaded portion 657. A clutch spring 656 is positioned around an unthreaded proximal portion 659 of the retractor 654. The clutch spring presses against a proximal end surface 655 of the threaded portion of the retractor 654. Proximal movement of the actuation element 112 after closure of the device 604 can result in overclosure or compression of the valve repair device or treatment device. The position of the unthreaded portion 657 is selected to prevent or inhibit over-retraction of the actuation element 112 and thereby prevent or inhibit over-closing of the valve repair device. That is, the externally threaded retractor 654 is no longer driven proximally when the externally threaded nut or retractor 654 reaches the unthreaded portion 657. The clutch spring is configured to bias the externally threaded retractor 654 distally (e.g., toward the threads of the internally threaded tube 652) when the externally threaded retractor 654 has reached the end of the threads of the internally threaded tube 652. Continued rotation of the knob 626 following disengagement of the external threads of the externally threaded retractor 654 and the internal threads of the internally threaded tube 652 results in an audible indication that the device is in a closed position. The biasing of the externally threaded retractor 654 can also reduce slop in the threaded connection, thereby improving stability of the paddle angle. The biasing of the externally threaded retractor 654 towards the internal threads of the internally threaded tube 652 ensures that the externally threaded retractor 654 will be re-engaged by the internally threaded tube 652 when the knob 626 is rotated in an opposite direction that corresponds to advancement of the actuation element 112.

As shown in FIGS. 79, 80, and 81, the externally threaded retractor 654 includes a central passage 683. A crimp assembly 668 is coupled in the passage 683. The crimp assembly attaches the actuation element 112 (e.g., actuation shaft, actuation rod, actuation tube, actuation wire, etc.) that is in an actuation tube 669 to the paddle release knob 630. The crimp assembly 668 can take a variety of different forms. In the illustrated example, the crimp assembly 668 comprises a collet 687 and a nut 670. The collet 687 includes external threads and the nut includes internal threads. The collet and nut each have a central bore that is sized to receive the actuation tube 669 which surrounds the actuation element 112. The actuation element 112 can be fixed to the actuation tube 669 near the proximal end of the actuation tube 669. The actuation tube 669 is fixed to the crimp assembly 668, which is fixed to the externally threaded retractor 654. The external threads on the outer surface of the collet 687 engage with the internal threads of the nut 670. When tightened, the collet 687 and nut 670 fix the crimp assembly to both the actuation tube 669 and a shoulder 689 at the proximal end of the retractor 654. The nut is positioned within the central bore 683 of the externally threaded retractor 654. A spring 672 is also positioned within the central bore 683 of the externally threaded retractor 654 such that the nut 670 is biased against the shoulder 689. The positioning of the nut 670 against the shoulder 689 allows the collet 687 to be easily attached to the nut 670.

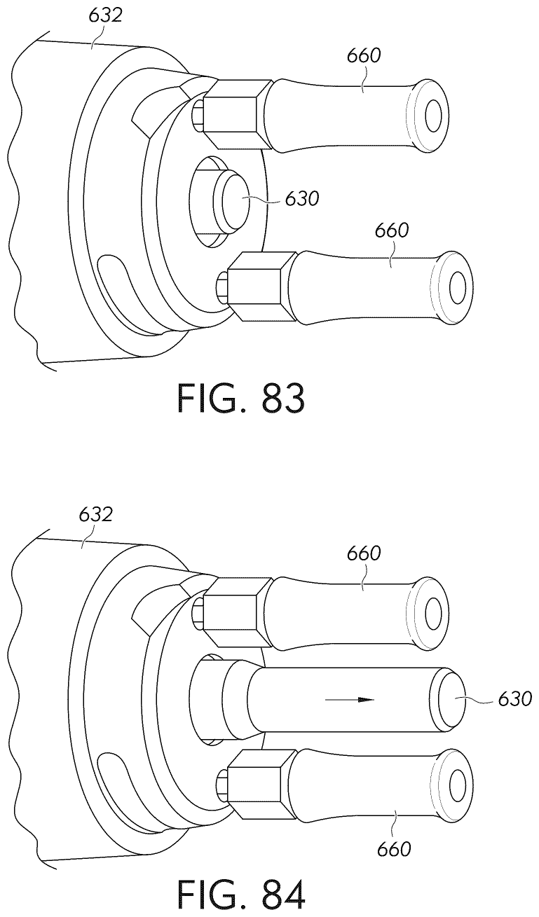

In some implementations, in use, the knob 626 is rotated, which rotates the internally threaded tube 652 to rotate with respect to the housing 632, thereby driving the externally threaded retractor 654 axially. The axial movement of the externally threaded retractor 654 causes axial movement of the actuation element 112, which moves the device between a fully elongated configuration, an open configuration, and a closed configuration. Axial movement of the externally threaded retractor 654 also causes axial movement of the release knob 630 between a proximal, or extended, position (shown in FIG. 84) and a distal, or retracted, position (shown in FIG. 83). Accordingly, in implementations, the axial position of the release knob 630 with respect to the housing 632 is a visual indicator of the configuration of the device 604.

When the device 604 is in a closed configuration (e.g., shown in FIG. 21), the release knob 630 is in a proximal position, extending from the housing 632, as shown in FIG. 84. To release the device from the device/implant catheter assembly 610, the release knob 630 is rotated in an unscrewing or loosening direction, which rotates the crimp assembly 668, the actuation tube 669, and the actuation element 112. Rotation of the actuation element 112 extends down the length of the actuation element to the distal end portion 112b of the actuation element, thereby unscrewing the actuation element 112 from the cap 114 of the device 604.

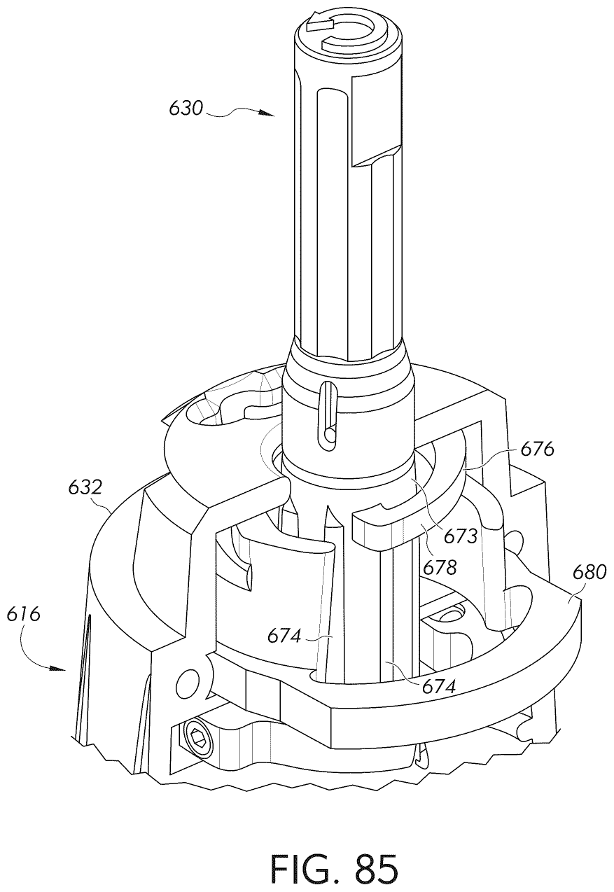

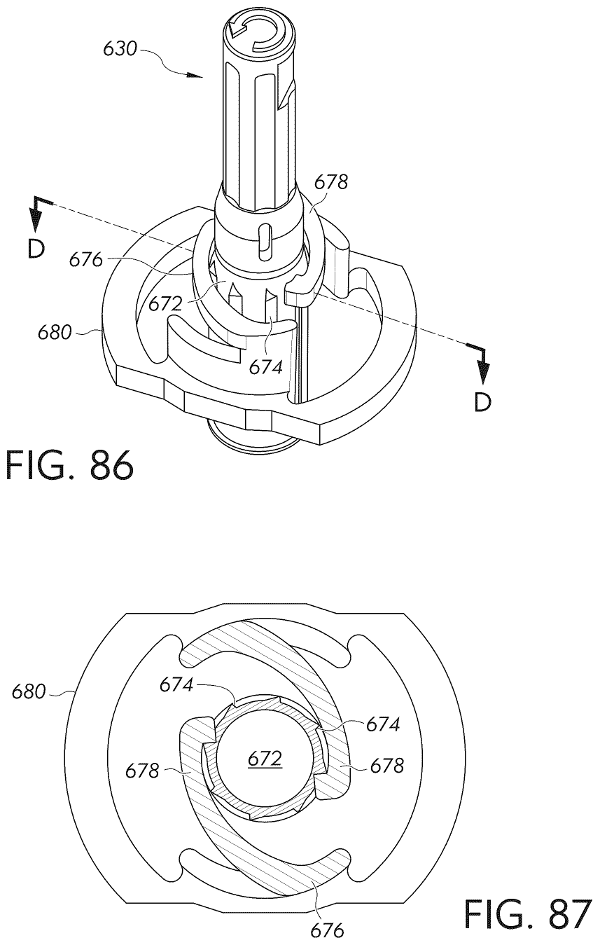

As shown in FIGS. 85-87, in implementations, the release knob 630 comprises an elongated shaft 673 and a plurality of teeth 674 extending from the outer surface of the elongated shaft 673. Each of the plurality of teeth 674 are uniform, but asymmetrical in shape, with each of the plurality of teeth 674 having a moderate slope on one edge and a much steeper slope on the other edge. A ratchet insert 676 is positioned within the housing 632 of the handle 616, between the elongated shaft 673 and the housing 632. The ratchet insert 676 includes one or more pawls 678 flexibly mounted to a ratchet frame 680. In implementations, the pawls 678 and the ratchet frame 680 are made from a unitary piece from a material that enables the pawls 678 to flex with respect to the ratchet frame 680.

Each of the pawls 678 is in contact with the outer surface of the elongated shaft 673. Accordingly, as the release knob 630 is rotated in the unscrewing or loosening direction, each pawl 678 slides up and over the edge of the tooth 674 with a moderate slope, and springs back into the area between the tooth and an adjacent tooth. In implementations, the springing back of the pawl into the area between teeth generates an audible indication that a tooth has been cleared. The edge of the tooth 674 with the steeper slope catches the pawl 678 prevents or inhibits the release knob 630 from being rotated in the opposite, tightening direction. Enabling a single direction of rotation of the release knob 630 prevents or inhibits torsion that can build up as a result of the torquing of the actuation element 112 during release of the device 604 from turning the release knob 630 back in the tightening direction.

Once the actuation element 112 is decoupled from the device 604, the actuation element 112 can be withdrawn into the device/implant catheter assembly 610, and the device/implant catheter assembly 610 can be withdrawn through the steerable catheter assembly 608 and the delivery catheter assembly 606.

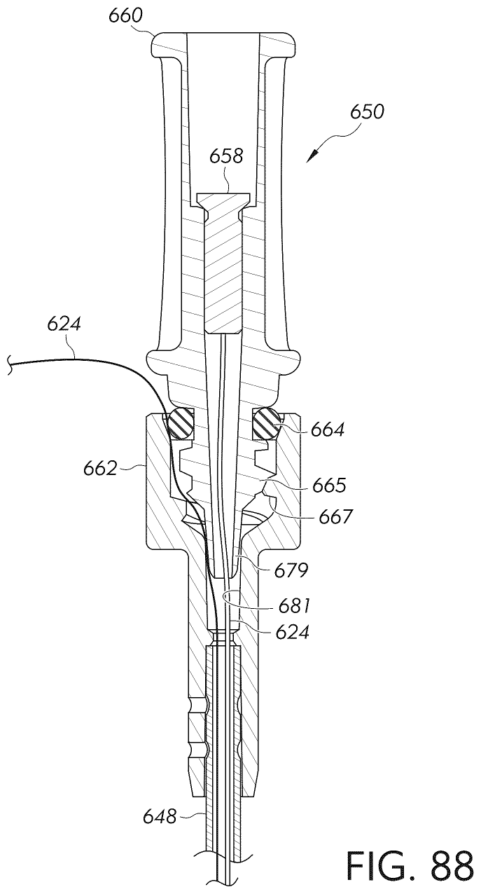

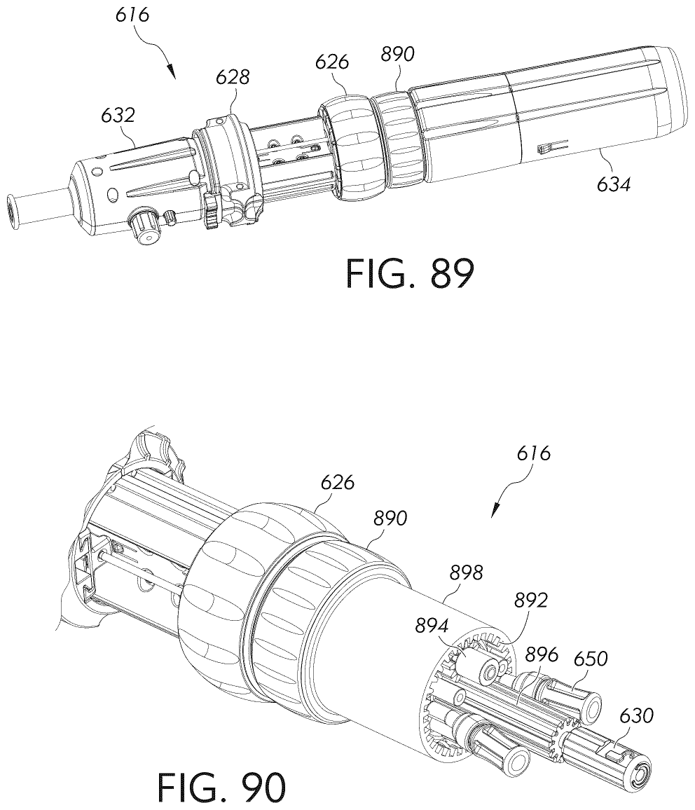

In implementations, a cap 634 (shown in FIG. 89) is removably couplable to the proximal end of the handle to cover the release knob 630 and the suture locks 650. Accordingly, the cap 634 can be positioned over the release knob 630 and suture locks 650 to prevent or inhibit the release knob 630 and suture locks 650 from being accidentally contacted or caught on something during manipulation of the device and removed to access the release knob 630 and suture locks 650.

In implementations, the handle 616 shown and described in FIGS. 77-88 can be modified to include the width control elements described hereinabove (e.g., paddle width control 1628 in FIGS. 70 and 71). As described above, the width control element or paddle width control element can take any one of a variety of forms and, in the implementation depicted in FIGS. 89-99, the paddle width control element is in the form of a paddle width control knob 890. In the implementation depicted in FIGS. 89-99, the paddle width control knob 890 is configured as a rotatable knob that rotates about a central axis extending through the handle 616 without interfering with the clasp control tubes and/or clasp control lines 624 (shown in FIGS. 77 and 78) that extend to the suture locks 650 positioned at the end of the handle 616.

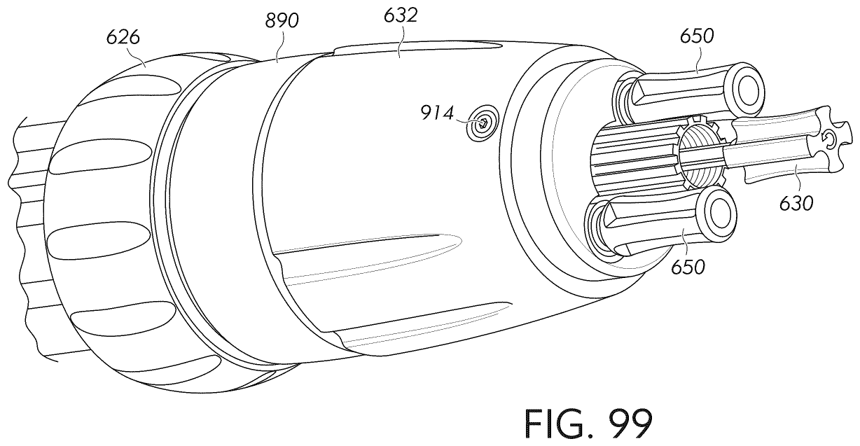

In implementations, such as the implementation depicted in FIG. 90, the suture locks 650 are angled relative to the central axis to accommodate the components associated with the paddle width controls, while in some implementations, such as the implementation depicted in FIG. 99, the suture locks 650 are parallel to the central axis. Accordingly, it should be appreciated that various modifications can be made to the handle 616 to incorporate the paddle width controls.

FIG. 90 illustrates various components of example anchor width controls or paddle width controls incorporated into the handle 616. As shown in FIG. 90, the width control or knob 890 is coupled to a planetary gearbox that includes a ring gear 892, a pair of planet gears 894 (one shown in FIG. 90), and an elongated central gear 896. Accordingly, rotation of the width control knob 890 with respect to the handle 616 is effective to cause rotation of the elongated central gear 896, which in turn is effective to move the width adjustment element (not shown in FIG. 90) in an axial direction, thereby adjusting the width of the anchors or paddles, as described hereinabove.

The ring gear 892 is housed within a barrel 898 that is fixed to the width control or knob 890 such that rotation of the width control or knob 890 turns the barrel 898 and the ring gear 892 fixed within the barrel 898. In the illustrated example, the axle of each planet gear 894 is fixed. Teeth of the ring gear 892 engage with teeth of the pair of planet gears 894 such that rotation of the ring gear 892 causes rotation of the planet gears 894 on their fixed axles. The teeth of the pair of planet gears 894 also engage with teeth of the elongated central gear 896 such that rotation of the pair of planet gears 894 causes rotation of the elongated central gear 896.

In the implementation shown in FIG. 90, the paddle release knob 630 extends from the end of the elongated central gear 896. In implementations in which the handle 616 includes the paddle width control knob 890, the paddle release knob 630 can function as the control element connection knob 7308 (shown and described in FIGS. 73-75) to enable the device to be disengaged from the implant delivery system that includes the handle 616.

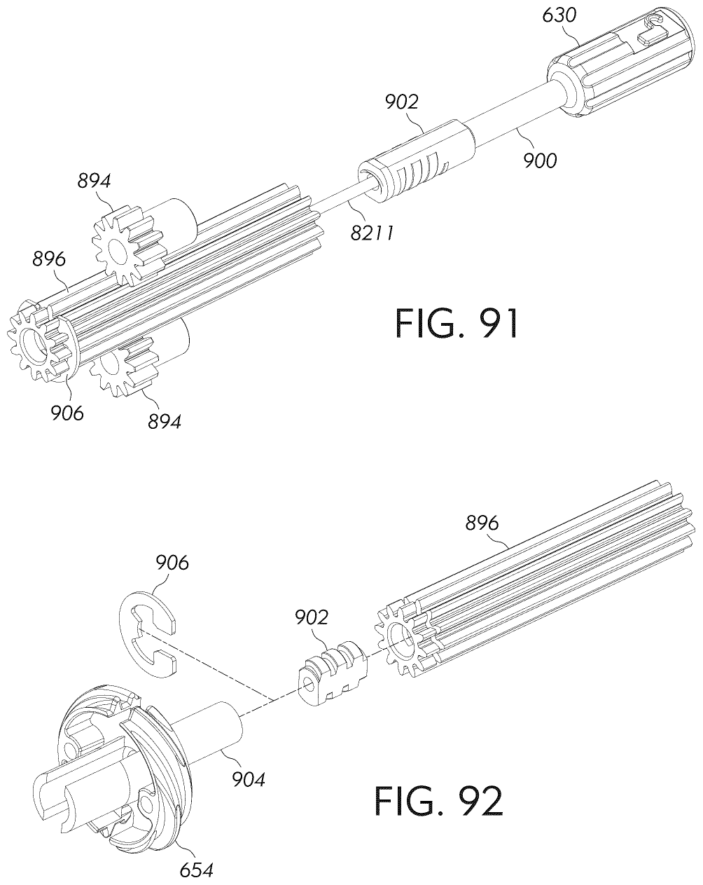

FIG. 91 shows the planet gears 894 and the elongated central gear 896 in greater detail. As shown in FIG. 91, the paddle release knob 630 is disposed at one end of a shaft 900. A second end of the shaft 900 is coupled to a follower 902. The follower 902 includes external threading that is configured to engage with internal threads of the elongated central gear 896. However, the follower 902 is rotationally fixed such that as the elongated central gear 896 is rotated, the follower 902 is axially driven with respect to the elongated central gear 896. The follower 902 is further fixedly coupled to the width adjustment element 8211. Accordingly, axial movement of the follower 902 results in axial movement of the width adjustment element 8211, which is effective to adjust the width of the paddles, as described above.

The elongated central gear 896 is positioned within the handle 616 proximate the externally threaded retractor 654 and receives a shaft 904 extending from one end of the externally threaded retractor 654. The shaft 904 is rotatably coupled to the elongated central gear 896 adjacent the follower 902. The width adjustment element 8211 extends through a central passage of the shaft 904 and the externally threaded retractor 654. A clip 906 axially fixes the shaft 904 with respect to the elongated central gear 896. As described above, the externally threaded retractor 654 is rotationally fixed relative to the housing of the handle 616 and is configured to open and close the device coupled to the handle 616 as it is driven axially. The elongated configuration allows the elongated central gear 896 to maintain meshing contact with the planet gears 894 as the elongated central gear moves axially with the externally threaded retractor 654. Rotation of the elongated central gear 896 is effective to drive the follower 902 axially with respect to the externally threaded retractor 654. The movement of the follower 902 relative to the externally threaded retractor 654 is effective to move the width adjustment element 8211 with respect to the actuation portion of the device, thereby adjusting the width of the paddles.

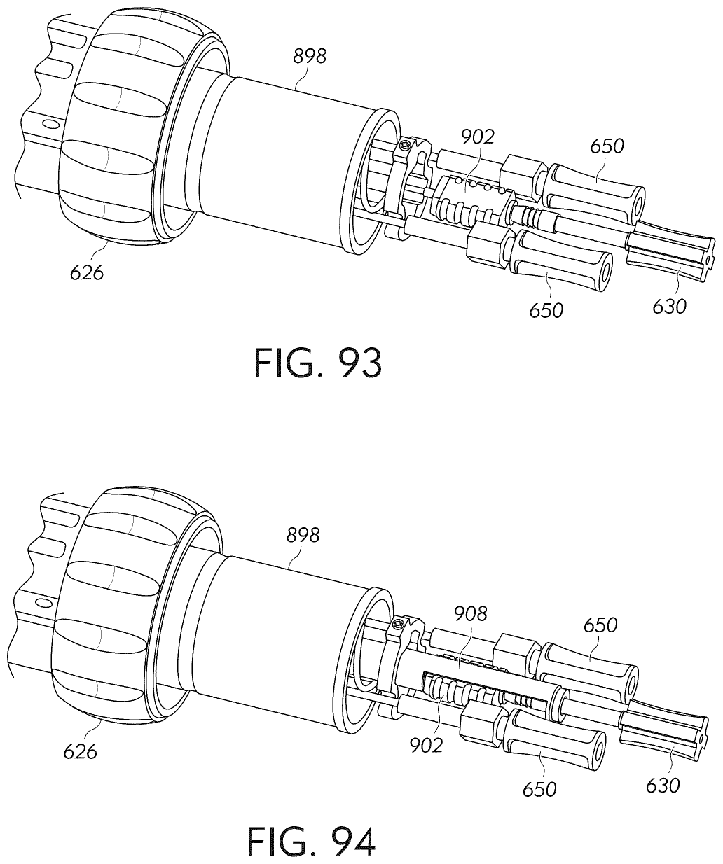

FIG. 93 shows the barrel 898 extending from the handle 616 and surrounding various other components housed within the handle 616. Extending from the end of the barrel 898, the follower 902 can be seen in position without being obstructed by the elongated central gear 896. As shown in FIG. 94, a guide 908 extends around the follower 902 and is configured to prevent or inhibit the follower 902 from rotating. The guide 908 is rotationally fixed with respect to the handle 616.

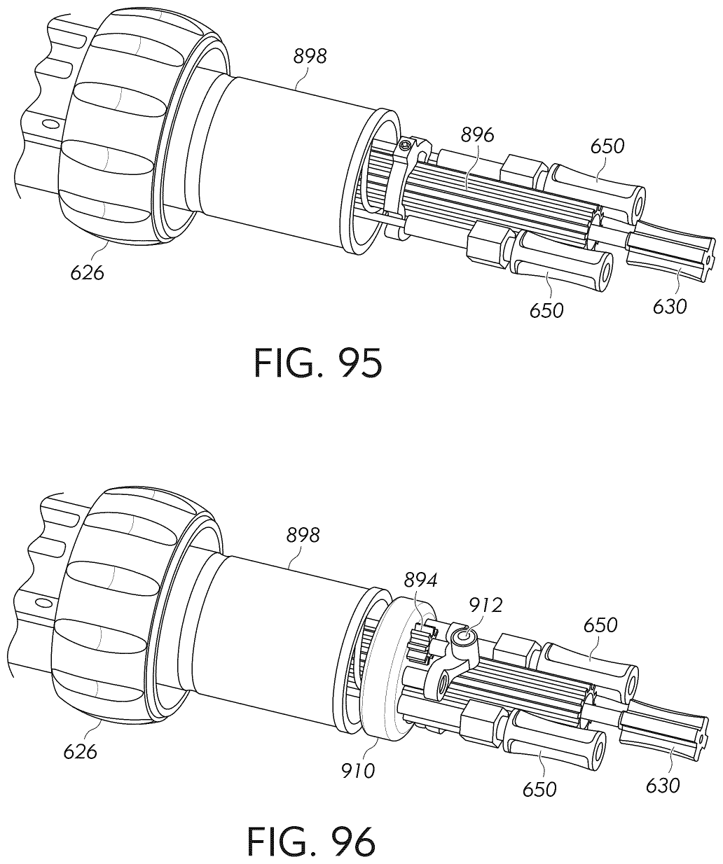

In FIG. 95, the elongated central gear 896 is shown extending from one end of the barrel 898. The guide 908 and the follower 902 extend through a central passage of the elongated central gear 896. As described above, rotation of the elongated central gear 896 is effective to drive the follower 902 axially, since rotation of the follower 902 is prevented or inhibited by the guide 908. The external teeth of the elongated central gear 896 engage with the teeth of the pair of planet gears 894, one of which is shown in FIG. 96.

FIG. 96 also shows a carrier 910, which is part of the planetary gearbox. The carrier 910 is fixedly coupled to a portion of the handle 616, such as the housing 632, and provides support for the axels and gears (e.g., the pair of planet gears 894). To facilitate coupling to the handle 616, the carrier 910 is attached to a coupler 912. In the implementation shown in the Figures, the coupler 912 is configured to receive a screw (e.g., screw 914 in FIG. 99). However, in some implementations, the coupler 912 can take other forms or can be omitted, provided that the carrier 910 is otherwise coupled to the handle 616.

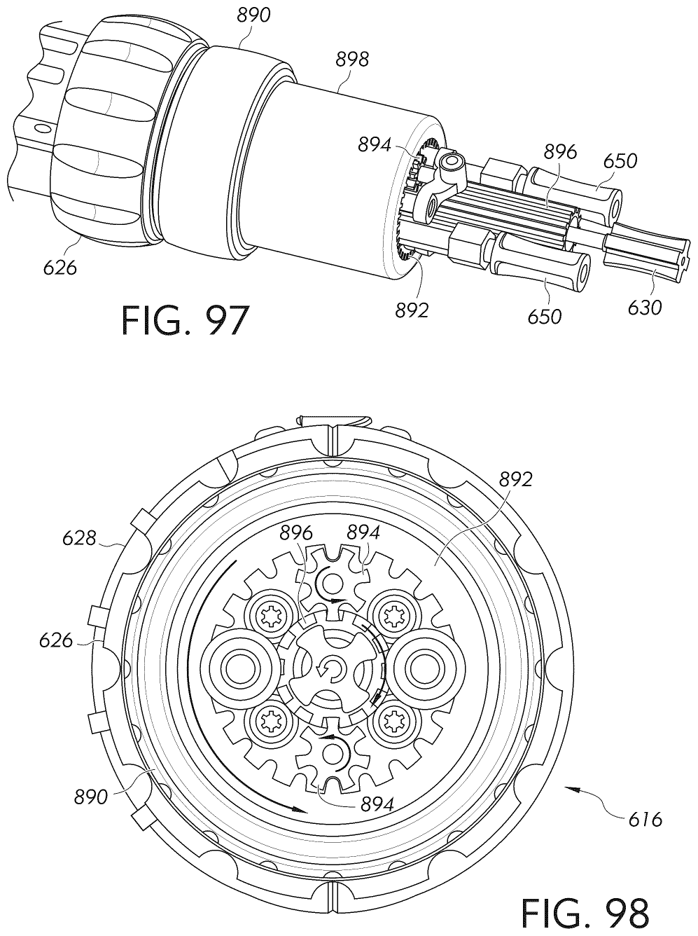

FIG. 97 shows the carrier and pair of planet gears in position within the barrel 898. The ring gear 892 is positioned within the barrel 898 to engage with the pair of planet gears 894. The elongated central gear 896 extends between and engages with the pair of planet gears 894.

FIG. 98 is an end view of the handle 616 showing the planetary gear box. More particularly, FIG. 98 shows the control or knob 626, clasp control member 628, and the paddle width control 890, in addition to the ring gear 892, the pair of planet gears 894 and the elongated central gear 896. As shown by the arrows, counter-clockwise rotation of the paddle width control 890 causes counter-clockwise rotation of the ring gear 892, which turns the pair of planet gears 894 counter-clockwise. The counterclockwise rotation of the pair of planet gears 894 rotates the elongated central gear 896 clockwise. As described above, the rotation of the elongated central gear 896 is effective to drive the width adjustment element 8211 axially, thereby adjusting the width of the paddles of the device.

FIG. 99 depicts the housing 632 positioned about the barrel 898. As described above, a screw 914 extends through the housing 632 and is received by the coupler 912 to fix the carrier 910 with respect to the handle 616. In implementations, the housing 632 includes features that enable the cap 634 (FIG. 89) to be coupled to the housing 632.

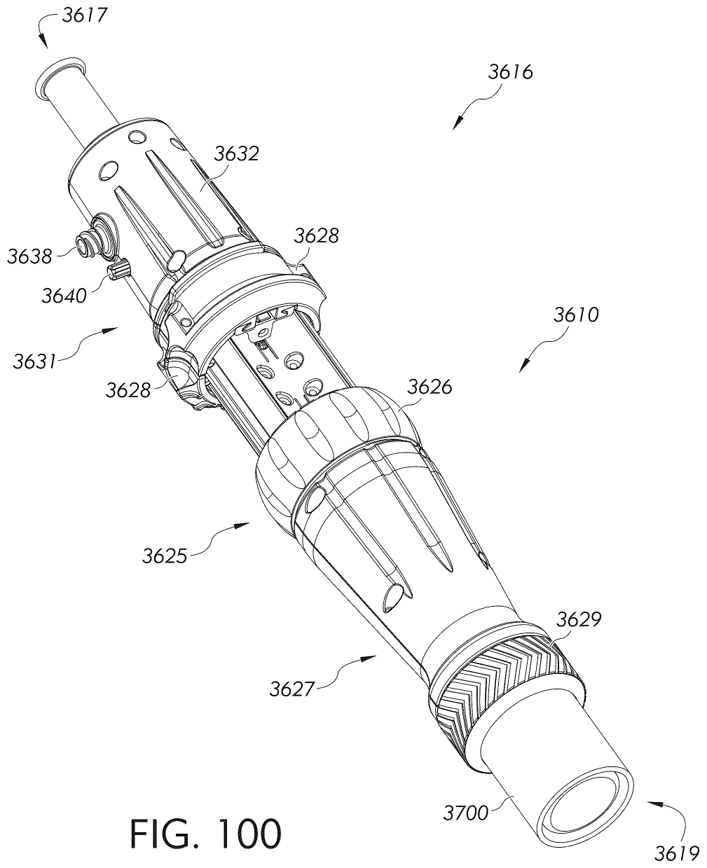

FIG. 100 illustrates an implementation of a handle 3616 of a device/implant catheter assembly 3610 (e.g., a control catheter assembly, a device control catheter assembly, an implant control catheter assembly, an implant delivery catheter assembly, etc.) and/or delivery system used to deliver a device or implant, such as the device 8200 (FIG. 67). While described regarding delivery of the device 8200, the handle 3616 can be used to deliver any suitable device or implant, such as any of the devices described herein. In the example of FIG. 100, the handle 3616 has a distal end 3617 and a proximal end 3619 opposite the distal end 3617. The handle 3616 can include some or all of a housing 3632, a paddle actuation portion 3625 having a paddle actuation control 3626, a clasp actuation portion 3631 having one or more clasp control members 3628, a paddle width control portion 3627 having a paddle width control 3629, and a release control 3630 (FIG. 101).

As shown in FIGS. 100-102 and discussed below, in some implementations of the handle 3616, the clasp actuation portion 3631, the paddle actuation portion 3625, the paddle width control portion 3627, and the release control 3630 are arranged is series in the handle 3616. For example, in some implementations, the clasp actuation portion 3631 is positioned distally from the paddle actuation portion 3625 which is positioned distally from the paddle width control portion 3627 which is positioned distally from the release control 3630 in series in the handle 3616.

The housing 3632 can be formed from various materials, including polymers such as polycarbonate, and can be formed as a unitary body (e.g., through injection molding) or fastened together in any one of a variety of manners, including fasteners, pins, adhesives, or the like. As shown in FIGS. 101-102, the housing 3632 comprises a plurality of lumens, through which the clasp actuation lines 3624, the actuation element (e.g., actuation rod, actuation tube, actuation wire, actuation shaft, etc.) 8102, and the width control element 8211 extend.

The handle 3616 can further comprise a flush port 3638 similar to the flush port 638 of the handle 616 of FIG. 77. Additional information regarding a flush port that can be used in the handle 3616 can be found in, for example, International Publication No. WO 2020/112622, the entire contents of which is incorporated by reference herein. The housing 3632 can additionally comprise an aperture for receiving a marking pin 3640 that can be removably coupled with the aperture of the housing 3632 for storage of the marking pin 3640. The aperture for the marking pin 3640 can be similar to the aperture for the marking pin 640 of the handle 616 of FIG. 77.

In some implementations, the paddle actuation control 3626 is configured to control the position of the actuation element 8102 relative to the handle 3616 and the catheters of delivery assembly. The paddle actuation control 3626 can be configured in a variety of ways, such as, for example, a knob, a button, a switch, one or more gears, etc. In some implementations, the paddle actuation control 3626 can be a lead screw mechanism that converts rotational motion to linear motion. For example, in some implementations, the paddle actuation control 3626 is configured to rotate about a longitudinal axis B of the handle 3616 to control movement of a mechanism (e.g., line, shaft, screw, internally threaded component, externally threaded component, lever, pulley, or any combination thereof) within the handle 3616 to control the position of the actuation element 8102. In some implementations, the paddle actuation control 3626 can include one or more linear actuators (e.g., electric actuator, manual actuator, pneumatic actuator, and/or hydraulic actuator) to control movement of a mechanism (e.g., line, shaft, screw, internally threaded component, externally threaded component, lever, pulley, or any combination thereof) within the handle 3616. A user interface, such as one or more actuation buttons, a touch screen, voice command interface or other suitable interface, can be provided (e.g., mounted on the handle housing and/or in the handle housing) and operatively connected to the linear actuators to allow a user to control the position of the actuation element 8102.

In the illustrated example of FIGS. 101-102, the paddle actuation control 3626 can be fixedly coupled to a first internally threaded tube 3652 positioned within the housing 3632 of the handle 3616. In some implementations, when the paddle actuation control 3626 is rotated about the longitudinal axis B of the handle 3616, the first internally threaded tube 3652 rotates with respect to the housing 3632. In some implementations, an externally threaded nut or retractor 3654 is positioned within, and engaged with, the first internally threaded tube 3652. The externally threaded nut or retractor 3654 can be rotationally fixed with respect to the housing 3632. The externally threaded nut or retractor 3654 can be rotationally fixed in a wide variety of different ways. In the example illustrated by FIG. 101, a pair of guide rods 3661 extend axially within the handle 3616, and each of the pair of guide rods 3661 is fixed to the housing 3632 at both ends of each of the guide rod 3661. In some implementations, a mounting bracket 3663 can optionally be included to couple the pair of guide rods 3661 to the housing 3632 at the distal end of each of the pair of guide rods 3661 and a collar 3666 couples the pair of guide rods 3661 to the housing 3632 at the proximal end of each of the pair of guide rods 3661. In some implementations, the system is configured such that as the first internally threaded tube 3652 is rotated, the externally threaded retractor 3654 is advanced linearly in an axial direction (i.e., distally and proximally) along the pair of guide rods 3661.

In some implementations, the externally threaded retractor 3654 is connected to a proximal end of the actuation element 8102 such that linear movement of the externally threaded retractor 3654 causes linear movement of the actuation element 8102, thereby moving the device 8200 between an open configuration and a closed configuration, as described herein. The proximal end of the actuation element 8102 can connect to the externally threaded retractor 3654 in any suitable manner. In the illustrated example, the proximal end of the actuation element 8102 is fixed to the externally threaded retractor 3654. The proximal end of the actuation element 8102 can be fixed to the externally threaded retractor 3654 in any suitable manner, such as by a ferrule 3612, or other crimped connection, positioned within a distal portion of a central longitudinal passage 3683 extending through the externally threaded retractor 3654. The translation of the rotational movement of the paddle actuation control 3626 into linear motion of the actuation element 8102 can result in improved control and precision, thereby leading to improved precision during the opening and closing of the device 8200.

The first internally threaded tube 3652 includes a proximal unthreaded portion 3657. Proximal movement of the actuation element 8102 after closure of the device 8200 can result in overclosure or compression of the device. The position of the unthreaded portion 3657 is selected to prevent or inhibit over-retraction of the actuation element 8102 and thereby prevent or inhibit over-closing of the device 8200. That is, the externally threaded retractor 3654 is no longer driven proximally when the externally threaded nut or retractor 3654 reaches the unthreaded portion 3657.

In some implementations, one or more optional springs 3656 or other suitable biasing elements, can be positioned to press against one or more proximally facing surfaces 3655 of the externally threaded retractor 3654. As shown in FIG. 101, the illustrated example includes a pair of springs 3656, with each spring 3656 received around a corresponding one of the pair of guide rods 3661. The springs 3656 are configured to bias the externally threaded retractor 3654 distally (e.g., toward the threads of the first internally threaded tube 3652) when the externally threaded retractor 654 has reached the end of the threads of the first internally threaded tube 3652. In some implementations, the system and handle are configured such that continued rotation of the paddle actuation control or knob 3626 following disengagement of the external threads of the externally threaded retractor 3654 and the internal threads of the first internally threaded tube 3652 results in an audible indication that the device is in a closed position. The biasing of the externally threaded retractor 3654 can also reduce slop in the threaded connection, thereby improving stability of the paddle angle. The biasing of the externally threaded retractor 3654 towards the internal threads of the first internally threaded tube 3652 ensures that the externally threaded retractor 3654 will be re-engaged by the first internally threaded tube 3652 when the paddle actuation knob 3626 is rotated in an opposite direction that corresponds to advancement of the actuation element 8102.

As shown in FIGS. 101-102, in some implementations, the externally threaded retractor 3654 includes a proximally extending stem 3682 having a radial shoulder 3684 in the central longitudinal passage 3683 that acts as a stop. In some implementations, the width adjustment element 8211 extends through the interior of the actuation element 8102, through the central longitudinal passage 3683, and out of a proximal end of the stem 3682. In some implementations, an annular stop 3686 is attached to the exterior of the width adjustment element 8211 within the central passage 3683, such as to the optional illustrated reinforcement tube 8212 that is fixed to the proximal portion of the width adjustment element. The annular stop 3686 is movable within the central longitudinal passage 3683 and configured to engage the radial shoulder 3684 to prevent or inhibit further proximal movement of the width adjustment element 8211 relative to the actuation element 8102. As such, when the annular stop 3686 reaches the radial shoulder 3684, the width adjustment element 8211, the externally threaded retractor 3654, and the actuation element 8102 all move together when the width adjustment element is moved further proximately.

In some implementations, the handle 3616 includes an extension or link 3688 configured to transfer proximal motion of the externally threaded retractor 3654 to the width control portion 3627. The link 3688 can be configured in a variety of ways. In the illustrated example of FIGS. 101-102, the link 3688 is formed as an elongated tube having a distal end 3690, a proximal end 3692 opposite the distal end 3690, and a central longitudinal passage 3694 extending through the link 3688. The width adjustment element 8211 extends through the central longitudinal passage 3694. The central longitudinal passage 3694 includes a counterbore 3696 at the distal end 3690 and a radial flange 3698 at the proximal end 3692. The counterbore 3696 is configured to receive the proximal end of the stem 3682 within the central longitudinal passage 3694. The radial flange 3698 defines a proximally facing engagement surface 3699.

The width control 3629 is configured to control the width of a portion of the anchors of the device, e.g., to control the width of the outer paddles 8120 of the device 8200. The width control 3629 can be configured in a variety of ways, such as, for example, a knob, a button, a switch, one or more gears, etc. In some implementations, the width control 3629 can be a lead screw mechanism that converts rotational motion to linear motion. For example, in some implementations, the width control 3629 is configured as a knob that rotates about the longitudinal axis B of the handle 3616 to control movement of a mechanism (e.g., line, shaft, screw, internally threaded component, externally threaded component, lever, pulley, or any combination thereof) within the handle 3616 to control the position of the width control element 8211. In some implementations, the width control 3629 can include one or more linear actuators (e.g., electric actuator, manual actuator, pneumatic actuator, and/or hydraulic actuator) to control movement of a mechanism (e.g., line, shaft, screw, internally threaded component, externally threaded component, lever, pulley, or any combination thereof) within the handle 3616. A user interface, such as one or more width control inputs, such as one or more actuation buttons, a touch screen, voice command interface or other suitable interface, can be provided (e.g., mounted on the handle housing and/or in the handle housing) and operatively connected to the linear actuators to allow a user to control the position of the width control element 8211.

As can be seen in the example in FIGS. 101-102, in some implementations, the width control 3629 is fixed to a rotatable barrel 3700 such that rotation of the width control 3629 rotates the barrel 3700 about the longitudinal axis B of the handle 3616. The barrel 3700 has a distal end 3702 rotatably coupled to the housing 3632 and a proximal end 3704 defining a cavity 3706 within which the release control 3630 is received. In some implementations, the barrel 3700 is operatively coupled to a second internally threaded tube 3708 positioned within the housing 3632 of the handle 3616 such that rotation of the barrel 3700 about the longitudinal axis B, via the width control 3629, rotates the second internally threaded tube 3708 relative to the housing 3632. In the illustrated example, the second internally threaded tube 3708 can also move axially relative to the housing 3632. The distal end of the second internally threaded tube 3708 abuts the proximally facing engagement surface 3699 of the radial flange 3698 of the link 3688 such that axial movement of the link 3688 moves the second internally threaded tube 3708 proximally. The proximal end of the second internally threaded tube 3708 abuts an end cap 3710.

The connection between the barrel 3700 and the second internally threaded tube 3708 can be any suitable connection that provides for rotational movement of the second internally threaded tube 3708 in response to rotation of the barrel 3700 while also allowing for axial movement of the second internally threaded tube 3708. In the illustrated example, the barrel 3700 connects to the second internally threaded tube 3708 via a splined connection.

The second internally threaded tube 3708 includes a central longitudinal passage 3712. Positioned within the central longitudinal passage 3712 is an externally threaded drive member 3714 and a cap 3715. The externally threaded drive member 3714 is configured to engage the internal threads of the second internally threaded tube 3708. The cap is positioned at a distal end of the externally threaded drive member 3714.

In some implementations, the externally threaded drive member 3714 is rotationally fixed with respect to the housing 3632. The externally threaded drive member 3714 can be rotationally fixed in a wide variety of different ways. In the example illustrated by FIGS. 101-102, a pair of guide rods 3716 extend axially through the central longitudinal passage 3712 of the second internally threaded tube 3708. Each of the pair of guide rods 3716 is fixed relative to the housing 3632 at both ends of the guide rod 3716. The pair of guide rods 3716 mount to the housing 3632 at the distal end of each of the pair of guide rods 3716 and the proximal end of each of the guide rods 3716 are fixed to the end cap 3710. Accordingly, as the barrel 3700 is rotated, the externally threaded drive member 3714 is advanced linearly in an axial direction (i.e., distally and proximally) along the pair of guide rods 3716. In some implementations, the externally threaded drive member 3714 can include longitudinal grooves (not shown) that receive the pair of guide rods 3716.

In the illustrated example, the externally threaded drive member 3714 is configured as a tube having a central longitudinal passage 3718 extending through the externally threaded drive member 3714. The central longitudinal passage 3718 includes a threaded nut 3720 adjacent a proximal end of the externally threaded drive member 3714. In some implementations, instead of a threaded nut, internal threads can be formed integrally within the passage 3718 of the externally threaded drive member 3714. A biasing member, such as a coil spring 3722 can be positioned within the central longitudinal passage 3718 between the threaded nut 3720 and the cap 3715 to bias the threaded nut 3720 toward the proximal end of the central longitudinal passage 3718.

The release control 3630 is configured to release the device 8200 from the device/implant catheter assembly 3610. The release control 3630 can be configured in a variety of ways, such as, for example, a knob, a button, a switch, one or more gears, etc. In some implementations, the release control 3630 can be a lead screw mechanism that converts rotational motion to linear motion. In some implementations, the release control 3630 can be a release button configured to release the device upon depression of the button. In some implementations, the release control 3630 can include one or more linear actuators (e.g., electric actuator, manual actuator, pneumatic actuator, and/or hydraulic actuator) to control movement of a mechanism (e.g., line, shaft, screw, internally threaded component, externally threaded component, lever, pulley, or any combination thereof within the handle 3616. A user interface, such as one or more buttons, a touch screen, voice command interface or other suitable interface, can be provided (e.g., mounted on the handle housing and/or in the handle housing) and operatively connected to the linear actuators to allow a user to release the device.

In the illustrated example, the release control 3630 is configured as a knob that rotates about the longitudinal axis B of the handle 3616. As can be seen in FIGS. 101-102, the release control 3630 is positioned at the proximal end 3619 of the handle 3616 and is received within the cavity 3706 of the barrel 3700. In some implementations, the handle 3616 includes a stem 3724 attached proximally to the release control 3630 and extending distally toward the externally threaded drive member 3714. A release screw 3726 is threadably received in the threaded nut 3720 in the externally threaded drive member 3714. The distal end of the stem 3724 is configured to engage a head of the release screw 3726 such that the release screw 3726 can be screwed into, or unscrewed from, the threaded nut 3720 by rotational movement of the release control 3630.

In some implementations, the width adjustment element 8211 extends through the length of the handle 3616 from the distal end 3617 to the release control 3630 where it is fixed such that movement of the release control 3630 results in corresponding movement of the width adjustment element 8211. In some implementations, at least a portion of the width adjustment element 8211 is received within a support tube 8212 (e.g., a hypotube) within the handle 3616. The support tube 8212 can be fixed at the release control 3630 along with the width adjustment element 8211. The support tube 8212 is configured to add strength to the portion of the width adjustment element 8211 within the handle 3616. The handle 3616 can also include a cover or cap 3728 over the release control 3630 to prevent or inhibit unintended release of the device 8200.

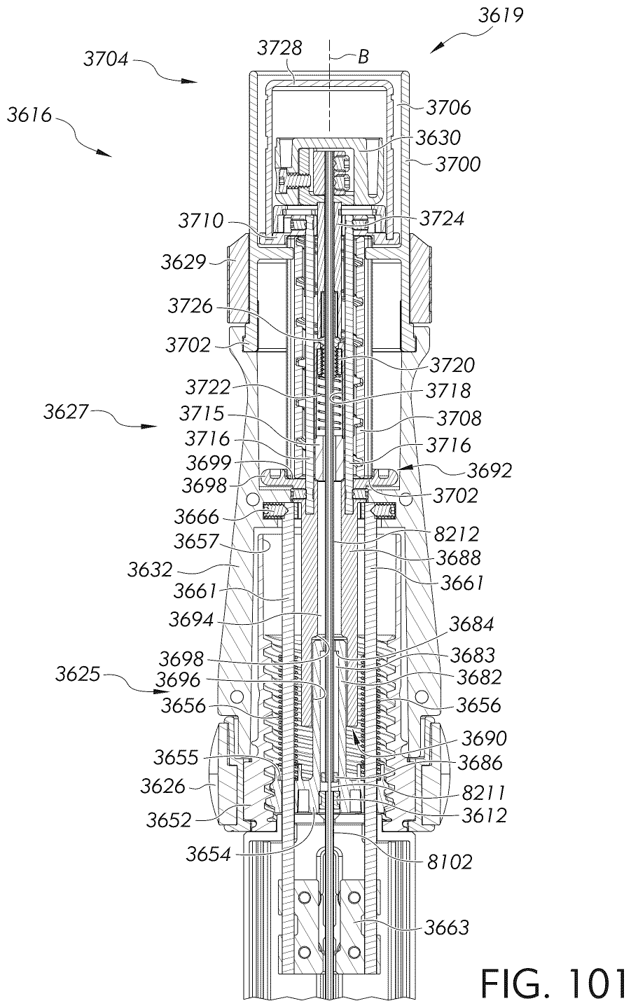

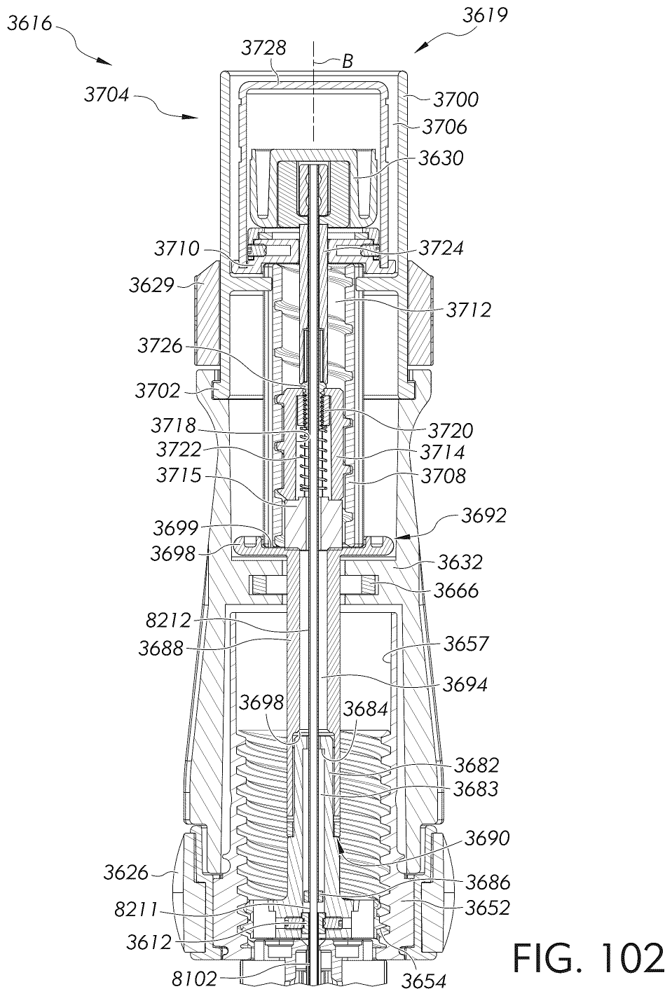

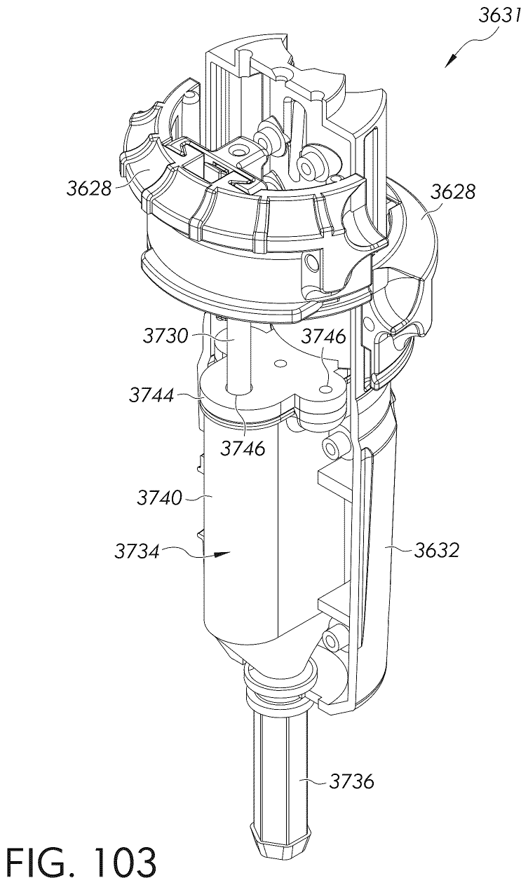

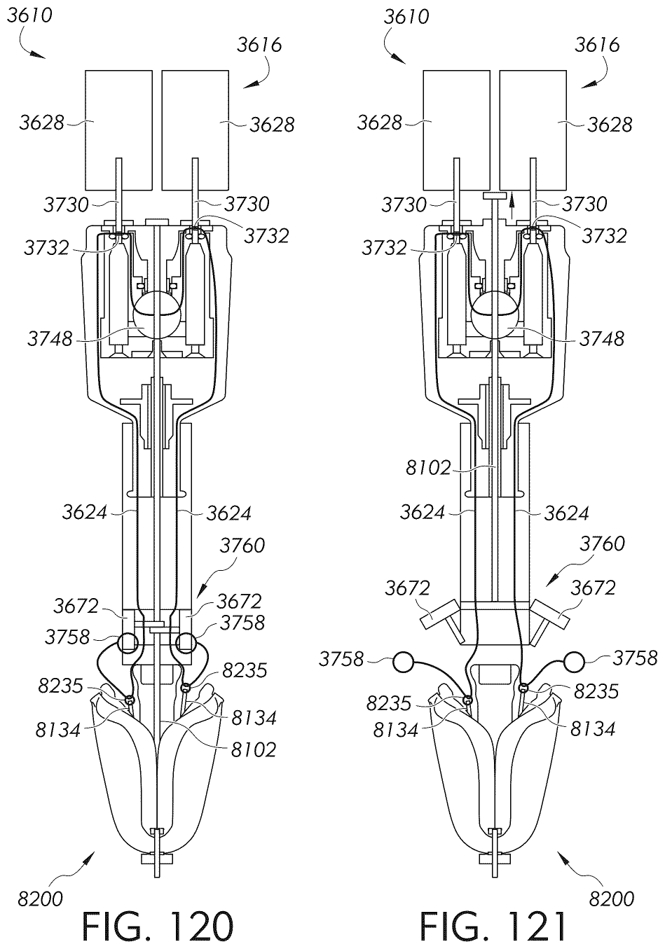

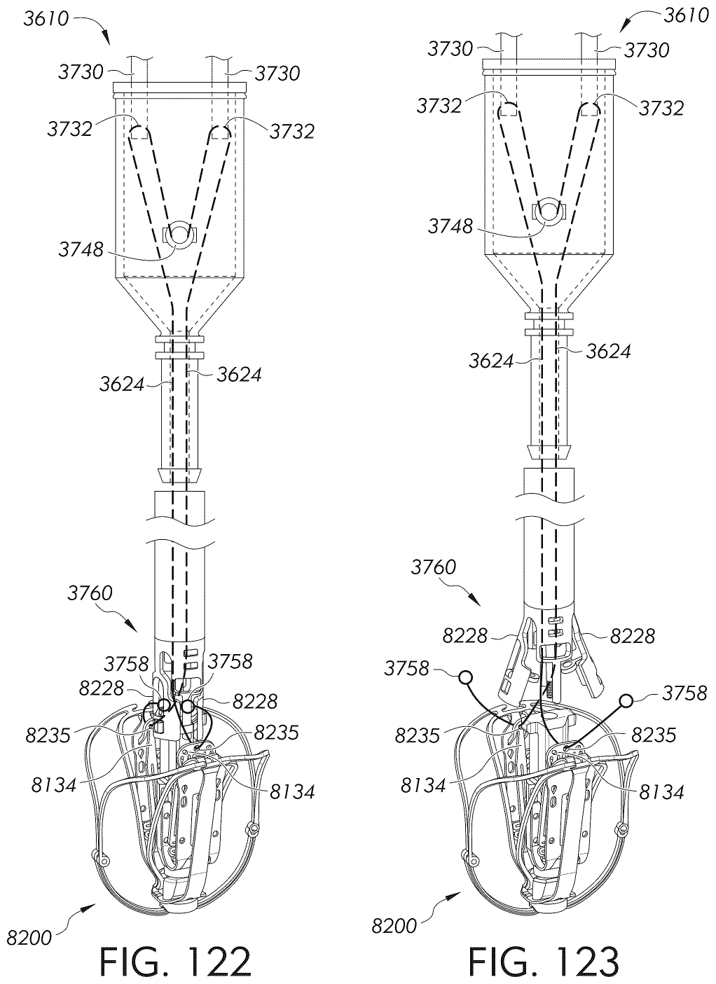

FIGS. 103-105 illustrate an example clasp actuation portion 3631 of the handle 3616. The illustrated clasp actuation portion 3631 includes the clasp control members 3628 which are configured to move the clasps of the device via clasp actuation lines (e.g., clasps 8134 of device 8200 via actuation lines 8102 as shown in FIGS. 122-124). The clasps 8134 can be configured in the same manner as any of the clasps described herein. The clasp actuation portion 3631 can be configured in a variety of ways. In some implementations, the device/implant catheter assembly 3610 includes two clasp actuation lines 3624 (FIGS. 120-121), each coupled to a corresponding clasp control member 3628 at the proximal end of the clasp actuation lines 3624. Each clasp control member 3628 can be, for example, an axially movable control or slider coupled to a corresponding clasp actuation line 3624 to axially move the clasp actuation line 3624 relative to the actuation element 8102. Each of the clasp control members 3628 can be operated independently of the other clasp control member such that each clasp actuation line 3624 is moved relative to the actuation element 8102 and the other clasp actuation line 3624. The clasp control members 3628 can also be fixed with respect to one another (e.g., locked) such that the clasp actuation lines 3624 are axially moved together relative to the actuation element 8102.

The clasp control members 3628 can be configured in a variety of ways, including similar to any clasp control member disclosed herein. In the implementation depicted in FIG. 103, the clasp control members 3628 are similar to the clasp control members 628 of FIG. 77, and thus the previous description of the clasp control members 3628 applies equally to the clasp control members 3628 shown in FIG. 103. The handle 3616 includes a pair of line-engaging members 3730 configured to engage the clasp actuation lines 3624 to move the lines in response to movement of the clasp control members 3628. The line-engaging members 3730 can be configured in a variety of ways. In the illustrated example, each line-engaging member 3730 is configured as an elongated rod having a proximal end fixed to a corresponding clasp control member 3628 and a distal end configured to engage a corresponding clasp actuation line 3624. In the illustrated example, the distal end of each line-engaging member 3730 includes a laterally extending passage 3732 through which a clasp actuation line 3624 can extend.

In some implementations, the handle 3616 can include a fluid body housing 3734 positioned within the housing 3632 of the handle 3616. The fluid body housing includes a distal stem 3736 defining a passage 3738, a main body portion 3740 defining an interior space 3742, and a proximal cap 3744. The proximal cap 3744 includes a plurality of orifices 3746 configured to allow the actuation element 8102 and the width adjustment element 8211 to extend through proximal cap 3744. In addition, each of the pair of line-engaging members 3730 extends distally from the clasp control members 3628 through a corresponding orifice 3746 in the proximal cap 3744 and into the interior space 3742. The main body portion 3740 further includes a line anchor 3748 to which the proximal ends of the clasp actuation lines 3624 attach. The line anchor 3748 can be configured in a variety of ways, including location, orientation, and anchoring means. In the illustrated example, the line anchor 3748 includes a passage 3750 from the interior space 3742 to the exterior of the main body portion 3740 and a boss 3752 surround the passage 3750 to which the clasp actuation lines 3624 attach.

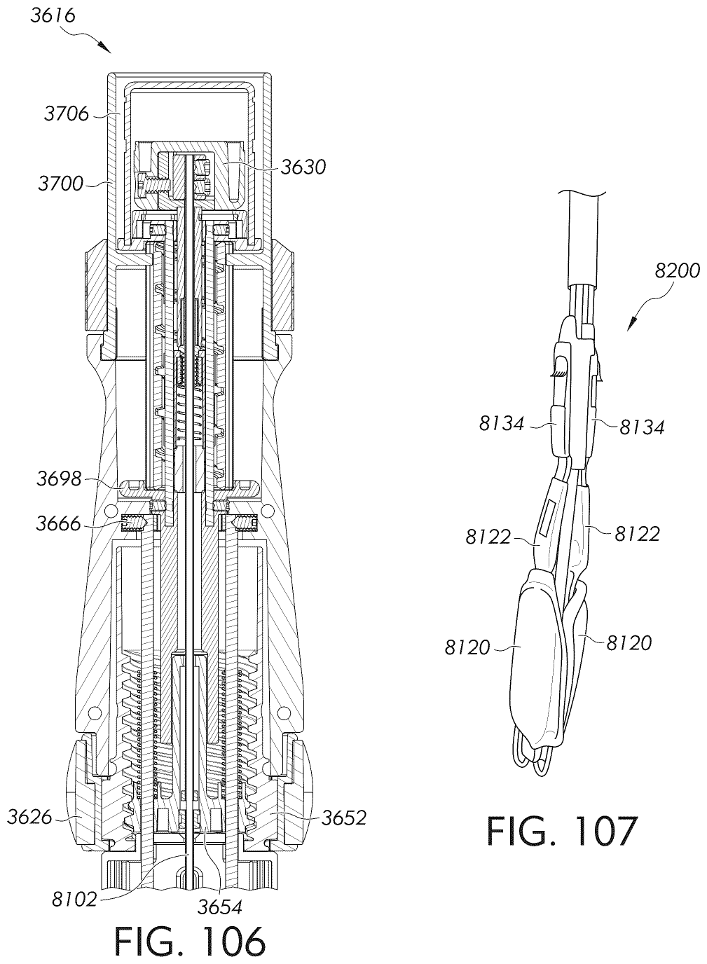

FIGS. 106-114 illustrate various stages of an example deployment of an example device 8200 by the handle 3616. The handle 3616 can be used to deploy any of the devices disclosed herein, such as the repair device or treatment device 8200, 100, 200, 300, 402, 9100, etc. As shown in FIG. 107, the device 8200 is in a fully elongated position where the outer paddles 8120 and the inner paddles 8122 are fully extended and clasps 8134 are in a fully open position. In addition, the outer paddles 8120 are in the expanded position.

As shown in FIG. 106, when the device 200 is in the fully open position, the externally threaded retractor 3654 is in a distal most position within the first internally threaded tube 3652, the radial flange 3698 is positioned near or adjacent the collar 3666, and the release control 3630 is positioned within the cavity 3706 of the barrel 3700.

To move the device 200 through the partially closed condition (FIG. 109) and to the fully closed condition (FIG. 110), the actuation element 8102 is retracted causing the paddles to move radially outward, as shown in FIG. 109 and, in the fully closed position, be positioned alongside the clasps 8134.

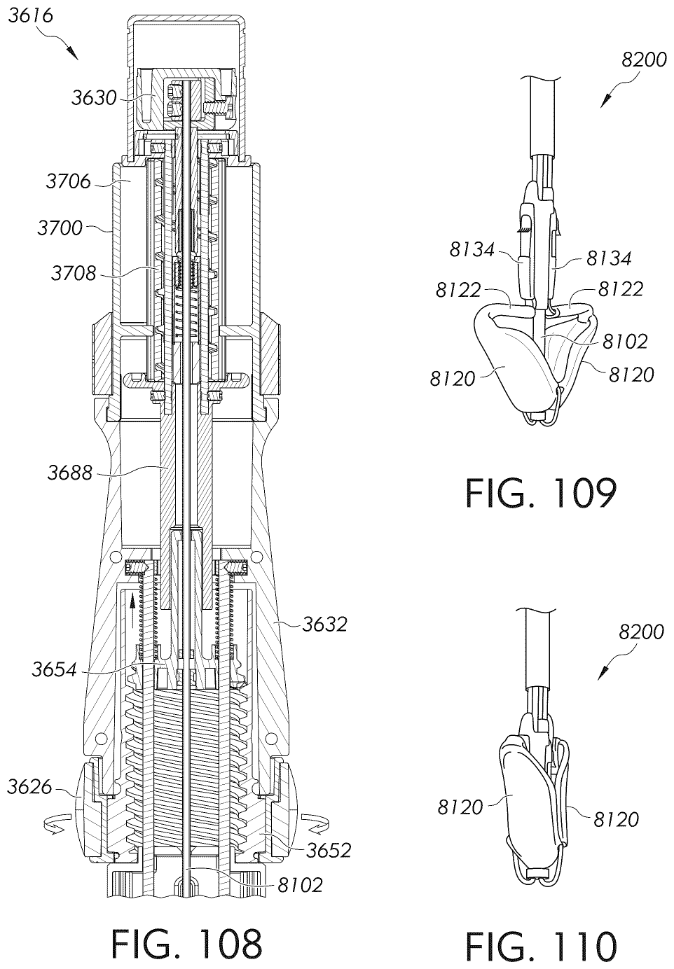

To retract the actuation element 8102, the paddle actuation control 3626 is rotated, which rotates the first internally threaded tube 3652 to rotate with respect to the housing 3632, thereby driving the externally threaded retractor 3654 axially in the proximal direction, as shown in FIG. 108. The axial movement of the externally threaded retractor 3654 causes axial movement of the actuation element 8102, which moves the device 8200 between the fully extended configuration (FIG. 107) and the closed configuration (FIG. 110).

Axial movement of the externally threaded retractor 3654 also causes axial movement of the second internally threaded tube 3708 via the link 3688. Axial movement of the second internally threaded tube 3708 causes axial movement of the release control 3630 between a position within the cavity 3706 of the barrel 3700 (shown in FIG. 106) to an extended position where the release control 3630 extends proximately from the cavity 3706 of the barrel 3700 (shown in FIG. 108). Accordingly, in implementations, the axial position of the release control 3630 with respect to the housing 3632 is a visual indicator of the configuration of the device 8200.

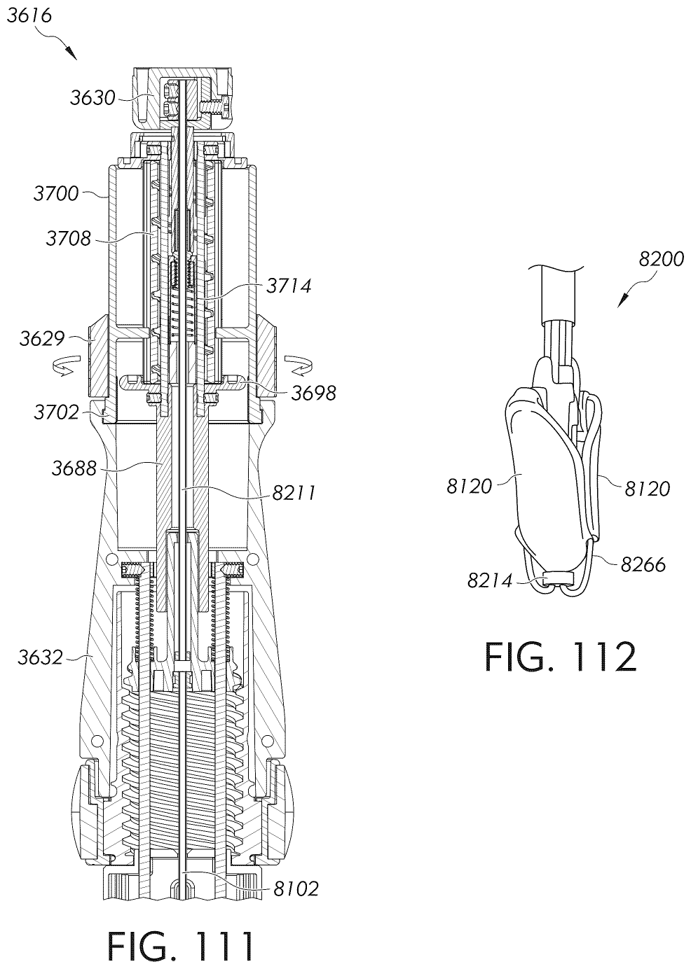

As shown in FIG. 112, the device 8200 is in the fully closed position and the outer paddles 8120 are in the expanded or widened position. In this position, as shown in FIG. 111, the link 3688, the release control 3630, the second internally threaded tube 3708, and the components within the second internally threaded tube 3708 (e.g., the externally threaded drive member 3714) have moved axially such that the radial flange 3698 of the link 3688 is position within the distal end 3702 of the barrel 3700.

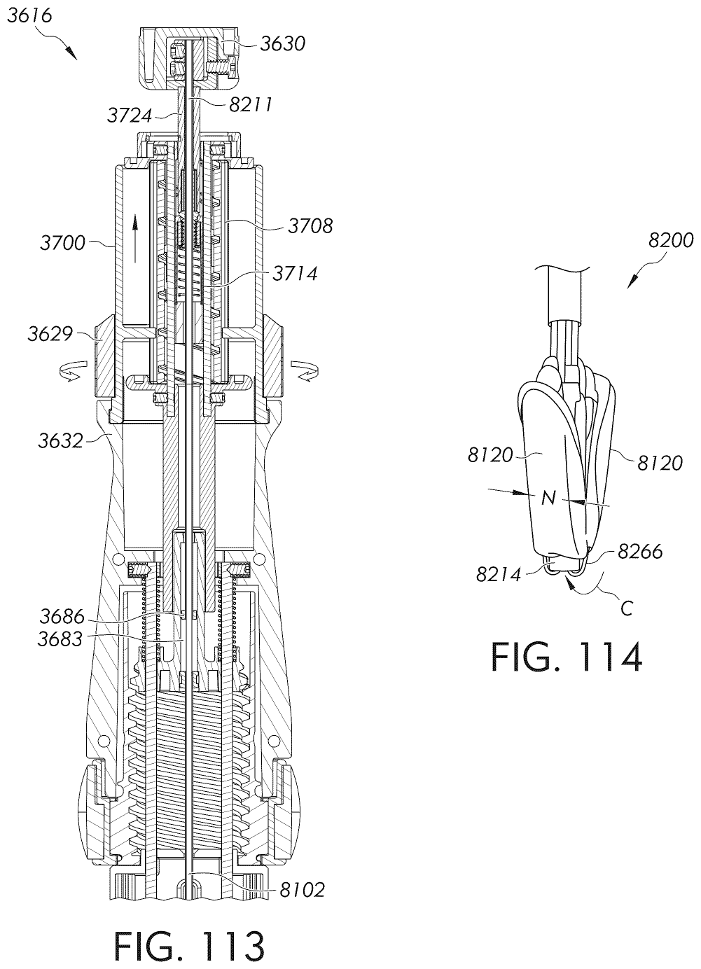

To move the outer paddles 8120 from the expanded position (FIG. 112) to the narrowed position (as shown by arrows N in FIG. 114), the width adjustment element 8211 is retracted. While FIGS. 111 and 113 illustrate the device 8200 in the fully closed position when the outer paddles 8120 are moved from the expanded position to the narrowed position, the outer paddles 8120 can also be moved between the expanded and narrowed positions while the device 8200 is in the fully extended position, the open position or any partially closed position. Retracting the width adjustment element 8211 causes the inner end 8968 of the paddle frames 8224 (FIG. 69) and portions of the connector 8266 to be pulled into the actuation cap 8214 (as shown by arrow C in FIG. 114).

To retract the width adjustment element 8211, the width control 3629 is rotated, which rotates the barrel 3700 and the second internally threaded tube 3708 relative to the housing 3632, thereby driving the externally threaded drive member 3714 axially in the proximal direction, as shown in FIG. 113. The axial movement of the externally threaded drive member 3714 causes axial movement of the release control 3630 via the stem 3724. Since the width adjustment element 8211 is attached to the release control 3630, the axial movement of the release control 3630 causes axial movement of the width adjustment element 8211 to move the outer paddles 8120 of the device 8200 to the narrowed position. With the axial movement of the width adjustment element 8211, the annular stop 3686 moves axially within the central longitudinal passage 3683 of the externally threaded retractor 3654 (e.g., axial movement relative to the externally threaded retractor 3654).

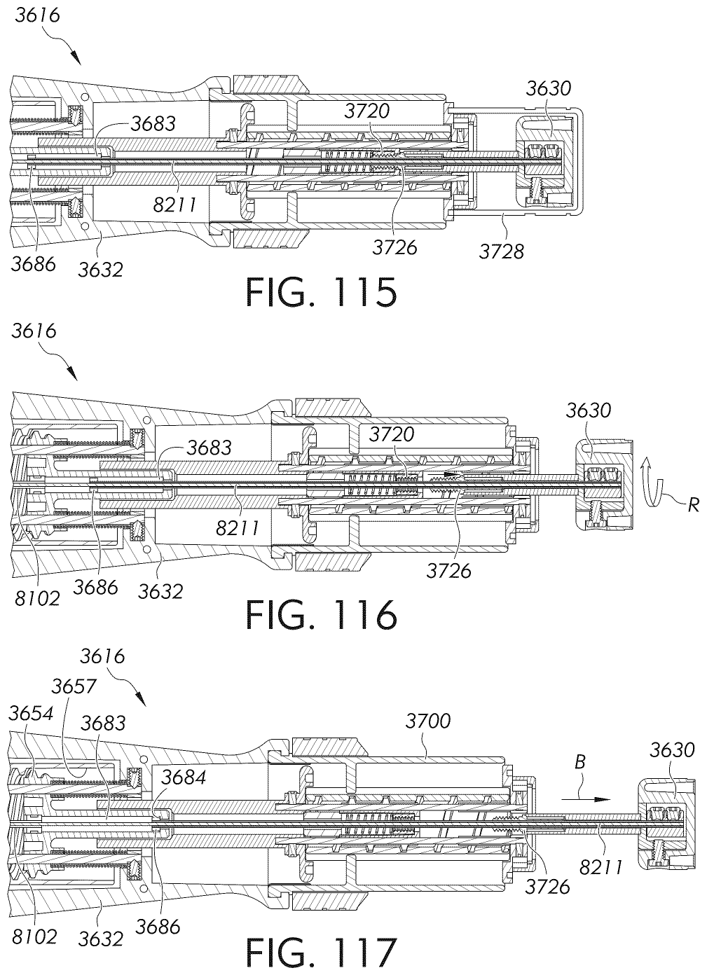

FIGS. 115-117 illustrate the handle 3616 at various stages of release of the device 8200. In FIG. 115, with the cover 3728 over the release control 3630, the handle 3616 is shown in the same position as in FIG. 113 (e.g., the device 8200 is in the fully closed position and the outer paddles 8120 are in the narrowed position). To release the device 8200, the width adjustment element 8211 is decoupled from the coupler 8972 of the device 8200 (FIG. 68-69). To decouple the width adjustment element 8211 from the coupler 8972, the cover 3728 is removed from over the release control 3630 and the release control 3630 is rotated, as shown by arrow R in FIG. 116. Rotating the release control 3630 rotates the release screw 3726 to unscrew the release screw 3726 from the threaded nut 3720 within the handle 3616 while also rotating the width adjustment element 8211 to unscrew the width adjustment element 8211 from the coupler 8972 within the device 8200. Unscrewing the release screw 3726 from the threaded nut 3720 also decouples the width adjustment element 8211 from the width control 3629.

Once the release screw 3726 and width adjustment element 8211 are unscrewed from the threaded nut 3720 and the coupler 8972, respectively, the release control 3630 can be pulled axially away from the barrel 3700, as shown by arrow B in FIG. 117. As the release control 3630 is pulled axially away, the width adjustment element 8211 is pulled with the release control 3630 resulting in the annular stop 3686 moving axially within the central longitudinal passage 3683.

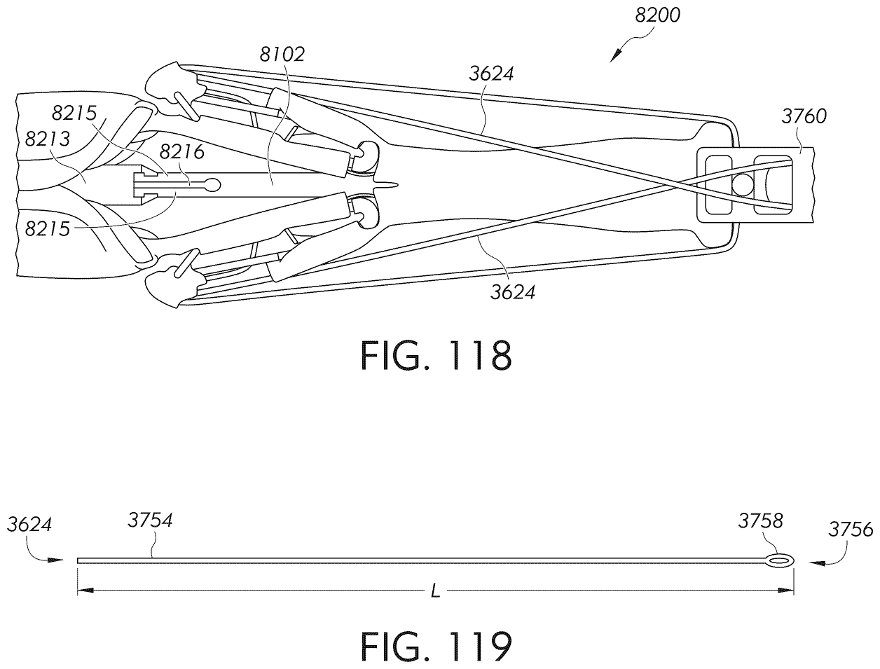

In the example illustrated in FIG. 118, the device 8200 is coupled to the device/implant catheter assembly by a capture mechanism 3760. The actuation element 8102 extends through the device/implant catheter assembly, through the capture mechanism 3760, and is attached to the device 8200 by a coupler 8213. In FIG. 118, the device 8200 is illustrated in the open condition to provide an unobstructed view of the coupler 8213. During release of the device 8200, however, the device 8200 will be in the fully closed position. In the illustrated example of FIG. 118, the distal end of the actuation element 8102 is received within the coupler 8213 and include two longitudinally extending legs 8215 separated by a slot 8216. The longitudinally extending legs 8215 can be configured (e.g., shape-set) to be biased inward toward each other. The width adjustment element 8211 (not shown in FIG. 118) extends coaxially within the actuation element 8102 and presses the longitudinally extending legs 8215 outward away from each other, which couples the actuation element 8102 to the coupler 8213. Various different capture mechanisms are possible that function in similar or different ways, e.g., threaded portions, snap-fit connections, locks, clasps, suture, loops, etc.

In some implementations, after the release screw 3726 and width adjustment element 8211 are unscrewed from the threaded nut 3720 and the coupler 8972, respectively, as the release control 3630 is pulled axially in the proximal direction, the width adjustment element 8211 is withdrawn relative to the actuation element 8102. Once the width adjustment element 8211 is withdrawn past the coupler 8213, the two longitudinally extending legs 8215 move inward to decouple the actuation element 8102 from the coupler 8213.

As shown in the example of FIG. 117, sufficient axial movement of the annular stop 3686 within the central longitudinal passage 3683 can result in the annular stop 3686 engaging the radial shoulder 3684 at the proximal end of the central longitudinal passage 3683. As a result, since the externally threaded retractor 3654 is in the proximal unthreaded portion 3657 of the first internally threaded tube 3652 (e.g., not engaged with the internal threads of the first internally threaded tube 3652), the externally threaded retractor 3654 is pulled with the release control 3630 and the width adjustment element 8211 with further axially movement of the release control 3630. Since the actuation element 8102 is attached to the externally threaded retractor 3654 and detached from the coupler 8213, the actuation element 8102 is pulled axially relative to the housing 3632 as well. The actuation element 8102 can be pulled until a proximal end of the actuation element clears the coupler 3760 to release the coupler from the device.

FIG. 119 illustrates an example clasp actuation line 3624. The clasp actuation line 3624 can be configured in a variety of ways, including, but not limited to, the materials used, the construction (e.g., braiding), the length, and the diameter. In the illustrated example of FIG. 119, the clasp actuation line 3624 includes a proximal or terminal end 3754, a distal end 3756 opposite the proximal terminal end 3754, and a closed opening 3758, such as a loop or eyelet, formed at the distal end 3756. The distance between the proximal terminal end 3754 and the distal end 3756 define a length L of the clasp actuation line 3624. In some implementations, the length Lis less than 100 inches or is less than 80 inches. In some implementations, the length is in the range of 65 inches to 75 inches, or 68 inches to 72 inches.

FIGS. 120-124 illustrate a simplified or schematic view (FIGS. 120-121) and a more detailed view (FIGS. 122-124) of the example device/implant catheter assembly 3610 coupled to the device 8200, in which each of the clasp actuation lines 3624 is coupled to a corresponding clasp control member 3628 positioned on the handle 3616. The device 8200 is coupled to the device/implant catheter assembly 3610 by the capture mechanism 3760 through which the actuation element 8102 (e.g., FIG. 120) extends.

In some implementations, the proximal end of each clasp actuation line 3624 is attached to the line anchor 3748. Each clasp actuation line 3624 extends from the line anchor 3748, passes through the passage 3732 at the distal end of each line-engaging member 3730, and extends distally through the device/implant catheter assembly 3610 (e.g., into a catheter/sheath) to the device 8200. At the device, each clasp actuation line 3624 extends through an opening 8235 in a corresponding one of the clasps 8134 of the device 8200 and the closed loop 3758 attaches to the capture mechanism 3760. The capture mechanism 3760 is configured to releasably attach the device 8200 to the device/implant catheter assembly 3610. The closed loops 3758 can attach to any suitable portion of the capture mechanism 3760 that allows the clasp actuation lines 3624 to be released when the capture mechanism 3760 releases the device 8200.

The capture mechanism 3760 can be configured in a variety of ways. In the example illustrated in FIGS. 120-124, the capture mechanism 3760 includes a plurality of flexible arms 8228 that are configured to pivot between a first or release configuration (FIGS. 121, 123-124) and a second or coupled configuration (FIGS. 120 and 122). The flexible arms 8228 are held in the coupled configuration by the actuation element 8102. When the actuation element 8102 is retracted from the capture mechanism 3760, the flexible arms 8228 pivot open to release the device 8200 from the device/implant catheter assembly 3610 (FIGS. 121 and 123-124). When the flexible arms 3672 pivot open, the closed loops 3758 at the distal ends 3756 of the clasp actuation lines 3624 are released from the capture mechanism 3760 allowing the clasp actuation lines 3624 to be withdrawn through the openings 8235 in the clasps 8134 and removed with the device/implant catheter assembly 3610.

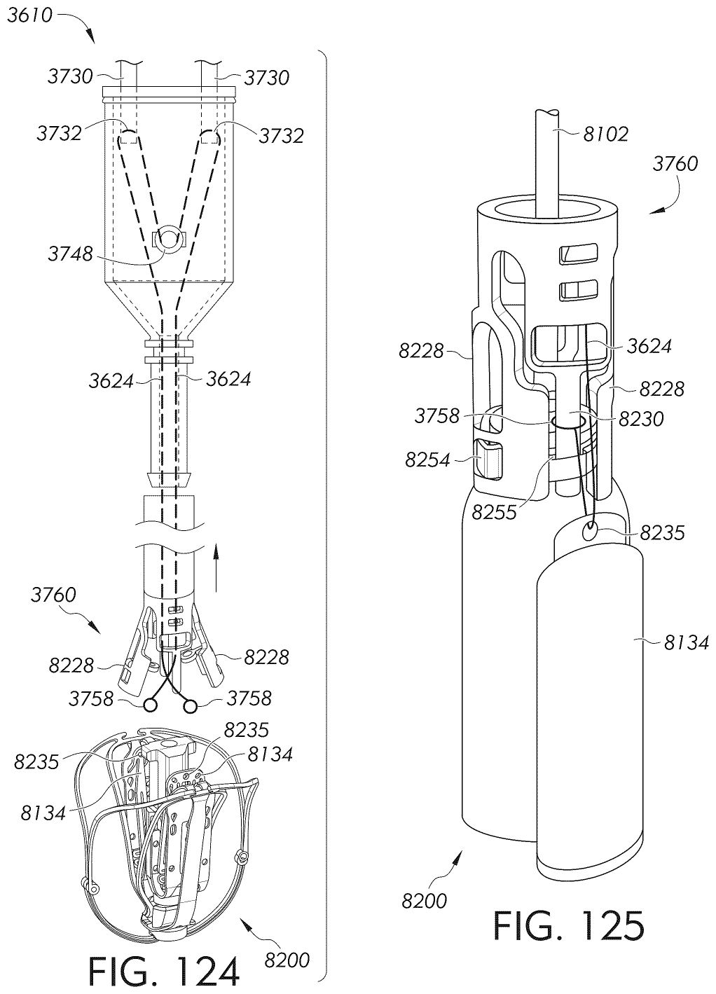

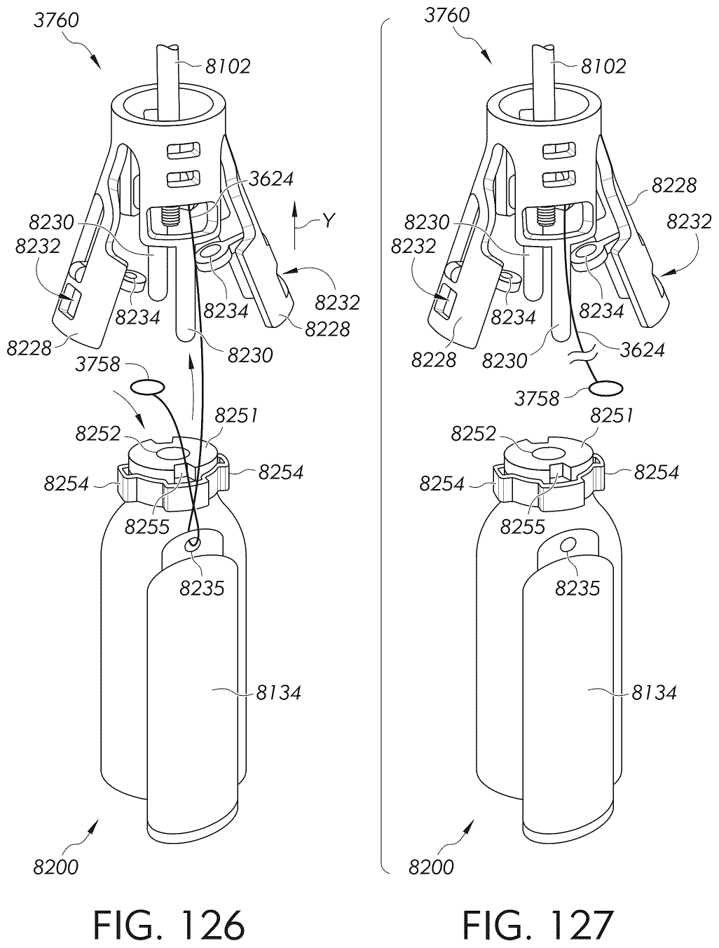

FIGS. 125-127 illustrate the example capture mechanism 3760 through various stages of releasing the device 8200. The capture mechanism 3760 can be releasably coupled to a proximal collar 8251 of the device 8200. The capture mechanism 3760 includes the plurality of flexible arms 8228 and a plurality of stabilizer members 8230. The flexible arms 8228 and the stabilizer members 8230 can be configured in a variety of ways. In the illustrated example, the stabilizer members 8230 are formed as longitudinally projecting fingers or prongs having a distal terminal end. The flexible arms 8228 can comprise apertures 8232 and eyelets 8234. The flexible arms 8228 can be configured to pivot between a first or release configuration (FIGS. 126-127) and a second or coupled configuration (FIG. 125). In the first configuration, the flexible arms 8228 extend radially outwardly relative to the stabilizer members 8230. In the second configuration, the flexible arms 8228 extend axially parallel to the stabilizer members 8230 and the eyelets 8234 radially overlap. The flexible arms 8228 can be configured (e.g., shape-set) to be biased to the first configuration.

The proximal collar 8251 of the device 8200 can include a central opening 8252 configured to slidably receive the actuation element 8102. The proximal collar 8251 can also include a plurality of bosses or projections 8254 and a plurality of guide openings 8255. The projections 8254 can extend radially outwardly and can be circumferentially offset (e.g., by about 90 degrees) relative to the guide openings 8255. The guide openings 8255 can be disposed radially outwardly from the central opening 8252. The projections 8254 and the guide openings 8255 of the proximal collar 8251 can be configured to releasably engage the capture mechanism 3760, as shown in FIG. 125, to attach the device 8200 to the device/implant catheter assembly. In particular, as shown in FIG. 125, when the capture mechanism 3760 is in the coupled configuration, the plurality of bosses or projections 8254 are received in the apertures 8232 of the flexible arms 8228, the plurality of stabilizer extensions or stabilizer members 8230 are received in the guide openings 8255, and the actuation element 8102 is received through the radially overlapping eyelets 8234. In addition, the clasp actuation lines 3624 are attached to the capture mechanism 3760 by receiving each of the stabilizer extensions/members 8230 through the closed loop 3758 of a corresponding clasp actuation line 3624 before the stabilizer extension/member 8230 is received in the guide openings 8255. Thus, the closed loop 3758 is captured between the proximal collar 8251 of the device 8200 and the capture mechanism 3760.

Referring to FIGS. 126-127, in some implementations, the device 8200 can be released from the device/implant catheter assembly by retracting the actuation shaft 8102 proximally relative to the capture mechanism 3760 such that the distal end portion of the actuation shaft 8102 withdraws from the eyelets 8234 of the capture mechanism 3760. This allows the flexible arms 8228 of the capture mechanism 3760 to move radially outwardly away from the projections 8254 of the proximal collar 8251. The stabilizer extensions or stabilizer members 8230 of the capture mechanism 3760 can then be withdrawn from the guide openings 8255 of the proximal collar 8251 by pulling the capture mechanism 3760 proximally, as shown by arrow Y in FIG. 126. With the stabilizer members 8230 withdrawn from the guide openings 8255, the closed loops 3758 are no longer trapped on the stabilizer extensions/members 8230 by the proximal collar 8251. As a result of pulling the capture mechanism 3760 proximally, the stabilizer members 8230 withdraw from the closed loops 3758 freeing the clasp actuation lines 3624. The clasp actuation lines 3624 can then be retracted through the opening 8235 in the clasps 8134 thereby releasing the device 8200 from the device/implant catheter assembly. as shown in FIG. 127.

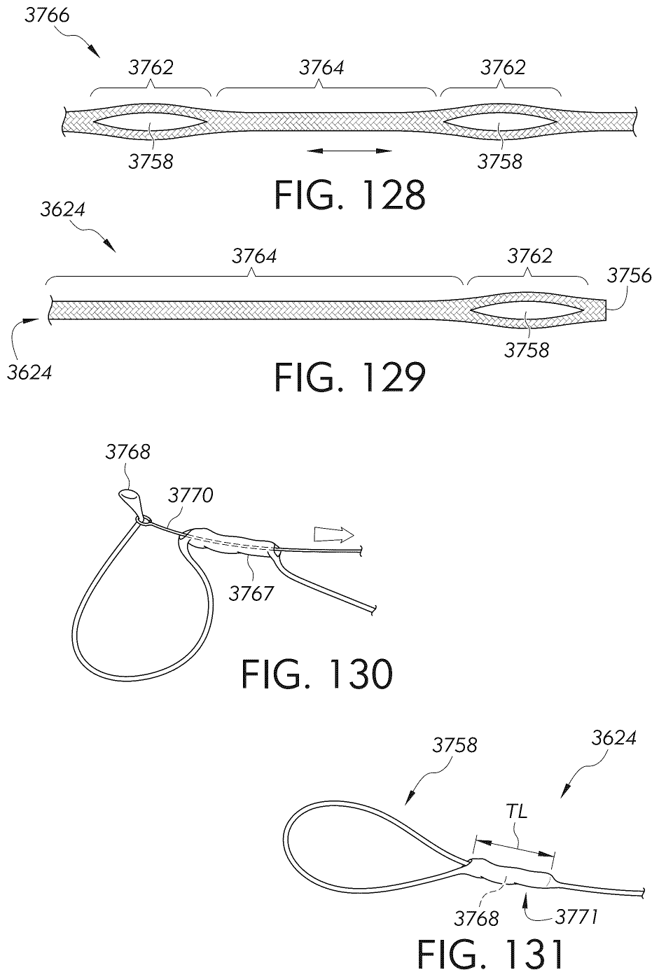

FIGS. 128-137 illustrate various implementations of the clasp actuation line 3624 of FIG. 119, including various configurations of the distal end 3756 of the clasp actuation line 3624. The clasp actuation line 3624 can be configured in a variety of ways, including, but not limited to, the materials used, the construction (e.g., braiding), the line length, the line diameter, shape, and the diameter of the closed loop 3758. In some implementations, the clasp actuation line 3624 is braided. The braided clasp actuation line 3624 can comprise any suitable number and size of yarns. For example, the clasp actuation line 3624 can be a braid having 4 to 100 ends using 10 to 400 dtex yarn. In some implementations, the clasp actuation line 3624 is a 16-end braid of 25 dtex yarn, is a 32-end braid of 10 dtex yarn, is an 8-end braid of 55 dtex yarn or is a 4-end braid of 110 dtex yarn.

In some implementations, the clasp actuation line 3624 has a tensile strength in the range of 20-100 N, or 30-70 N, or 30-50 N. In some implementations, the clasp actuation line 3624 is made from an ultra-high-molecular-weight polyethylene material, such as for example, Dyneema® fibers. In some implementations, the closed loop 3758 of the clasp actuation line 3624 has a nominal loop diameter in the range of 0.0275 inches to 0.0425 inches, or 0.03 inches to 0.04 inches, or 0.0325 inches to 0.0375 inches and a circumference in the range of 0.07 inches to 0.15 inches, or 0.09 inches to 0.13 inches. In some implementations, the clasp actuation line 3624 has a diameter in the range of 0.003 inches to 0.008 inches, or 0.004 inches to 0.006 inches. In some implementations, the clasp actuation line 3624 has a permanent deformation of less than 0.5% under a cyclic load of 0 to 10 N, or less than 0.3% under a cyclic load of 0 to 10 N.

Referring to FIGS. 128-129, the illustrated example of the clasp actuation line 3624 has an elongated braided body 3767 having a bifurcated section 3762 that forms the closed loop 3758 and a unitary section 3764 that forms the remainder of the clasp actuation line 3624. The bifurcated section 3762 can be formed as part of a braiding process, as opposed to forming a loop in a unitary section of a braided line after that line has been braided. In some implementations, the clasp actuation line 3624 can be made in a continuous process where multiple clasp actuation lines are formed in a single continuous braided line 3766 with alternating bifurcated and unitary sections and then cut into multiple clasp actuation lines. In some implementations, however, only a single clasp actuation line 3624 can be formed.

To form the clasp actuation line 3624 from a single continuous braided line 3766. A section of the single continuous braided line 3766 having the desired length and one bifurcated section 3762 is cut from the continuous braided line 3766 with the bifurcated section 3762 near an end (e.g., near where the continuous braided line 3766 is cut). The end near the bifurcated section 3762 forms the distal end 3756 of the clasp actuation line 3624. The distal end 3756 can be heat sealed or treated, or sealed or treated in another manner, to prevent or inhibit separation of the yarns at the distal end 3756.

Referring to FIGS. 130-131, the illustrated example of the clasp actuation line 3624 has an elongated braided body 3767 having a distal terminal end portion 3768 of the braided body 3767 tucked back into a more proximal portion of the braided body 3767 to form the closed loop 3758 and a tucked-in portion 3771. The terminal end portion 3768 can be tucked-in the braided body 3767 in a number of ways. In the illustrated example, a pull line or needle 3770 is tied to or connected to the terminal end portion 3768, as shown in FIG. 131. The pull line 3770 is inserted axially through a portion of the braided body 3767 proximal of the terminal end portion 3768. For example, a needle or other piercing device (not shown) can be attached to the pull line 3770 and used to thread the pull line 3770 axially along a portion of the braided body 3767 to form the closed loop 3758. In some implementations, the terminal end portion 3768 can attach directly to the needle or other piercing device. The tucked-in portion 3771 has a tucked-in length TL (FIG. 131) that can vary in different implementations. In some implementations, the tucked-in length TL is 0.25 inches or greater, is 0.5 inches or greater, is 1.0 inches or greater, or is 1.5 inches or greater. In some implementations, the tucked-in length is in the range of 0.25 inches to 1.5 inches.

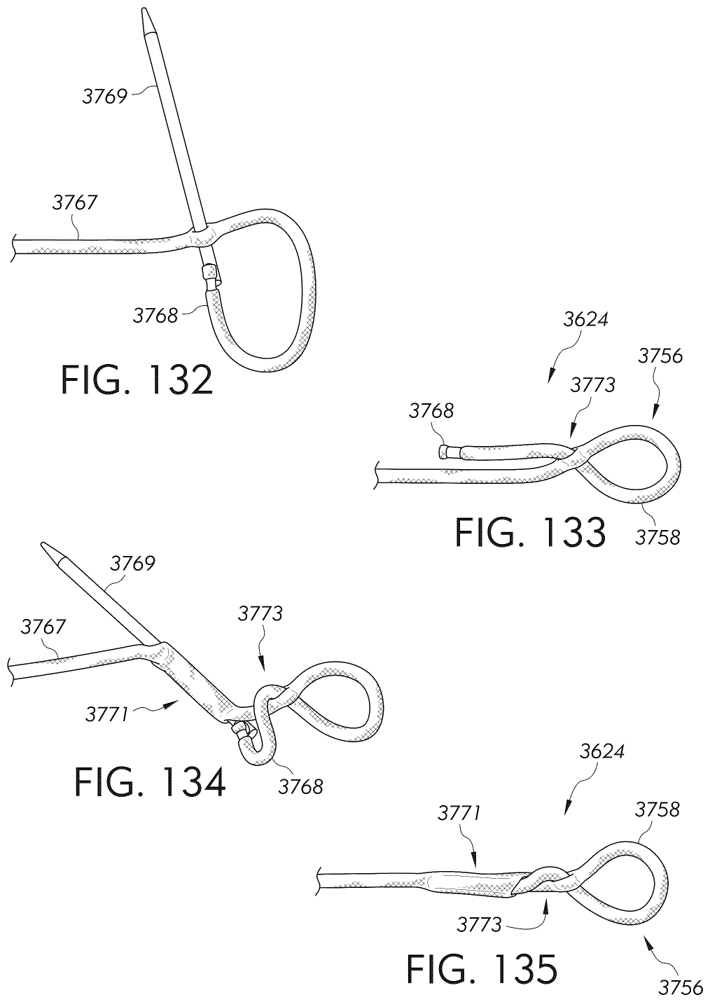

Referring to FIGS. 132-133, the illustrated example of the clasp actuation line 3624 has a braided body 3767 that uses a Brummel lock-splice, or other method of threading a distal terminal end portion 3768 of the braided body 3767 through a more proximal portion of the braided body 3767, to form a closed loop 3758 at a distal end 3756. The distal terminal end portion 3768 can be attached to a piercing device 3769, such as a splicing needle. As shown in FIG. 132, the piercing device 3769, with the distal terminal end portion 3768 attached, can be inserted laterally through a more proximal portion of the braided body 3767 to form a threaded portion 3773 proximal the closed loop 3758, as shown in FIG. 133. The distal terminal end portion 3768 can be threaded back and forth through the braided body 3767 at locations adjacent each other along the braided body 3767 any suitable number of times to lock the closed loop 3758 in place and secure the distal terminal end portion 3768. The exposed distal terminal end portion 3768 after being woven back and forth through the braided body 3767 can be cut-off, secured against the braided body 3767 in any suitable manner, or tucked-in a portion of the braided body 3767.

Referring to FIGS. 134-135, the illustrated example of the clasp actuation line 3624 has an elongated braided body 3767 and forms a closed loop 3758 at a distal end 3756 via a combination of weaving a distal terminal end portion 3768 of the braided body 3767 through a more proximal portion of the braided body 3767 to form a threaded portion 3773 and tucking the distal terminal end portion 3768 axially into the braided body 3767 to form a tucked-in portion 3771 (e.g., a combination of the methods described for the clasp actuation lines 3624 of FIGS. 130-133). In the illustrated example, the tucked-in portion 3771 is proximal the threaded portion 3773.

In particular, the distal terminal end portion 3768 can be attached to a piercing device 3769, such as a splicing needle. As shown in FIG. 134, the piercing device 3769, with the distal terminal end portion 3768 attached, can be inserted laterally through a more proximal portion of the braided body 3767 to form the closed loop 3758, as shown in FIG. 133. The distal terminal end portion 3768 can be threaded back and forth through the braided body 3767 at locations adjacent each other along the braided body 3767 any suitable number of times to lock the closed loop 3758 in place and secure the distal terminal end portion 3768. After weaving laterally through the braided body 3767 one or more times, the piercing device 3769 can be inserted axially through a portion of the braided body 3767 and pulled through to pull the distal terminal end portion 3768 into the braided body 3767 axially along a portion of the braided body 3767 to lock the closed loop 3758 in place and secure the distal terminal end portion 3768. The tucked-in portion 3771 has a tucked-in length TL that can vary in different implementations. In some implementations, the tucked-in length TL is 0.25 inches or greater, is 0.5 inches or greater, is 1.0 inches or greater, or is 1.5 inches or greater. In some implementations, the tucked-in length is in the range of 0.25 inches to 1.5 inches.

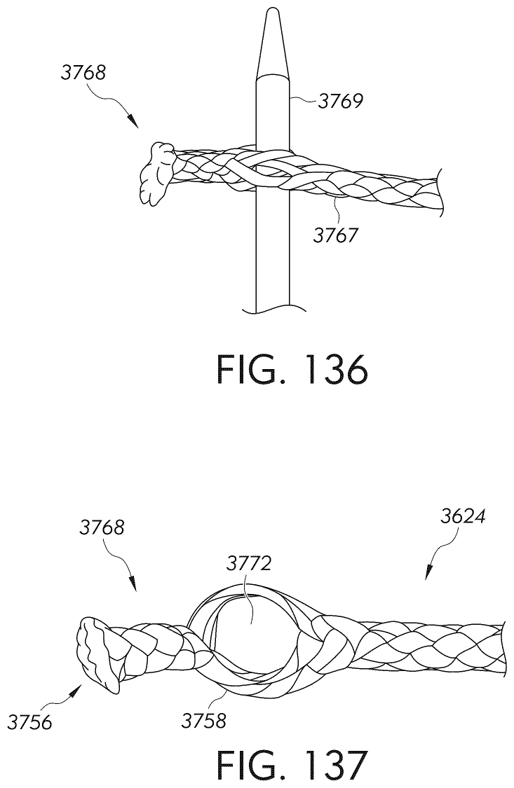

Referring to FIGS. 136-137, the illustrated example of the clasp actuation line 3624 has a braided body 3767 having a closed loop 3758 formed at a distal end portion 3768. In the illustrated example, the closed loop 3758 is formed by piercing a distal end portion 3768 laterally with any suitable piercing device 3769, such as a needle or pin, to create a lateral hole 3772 in the distal end portion 3768. Thus, the braided body 3767 is split by the piercing device 3769 to create the hole 3772 and form the closed loop 3758. The distal terminal end 3768 of the braided body can be heat sealed, or sealed in another manner, to prevent or inhibit separation of the yarns at the distal terminal end 3768.

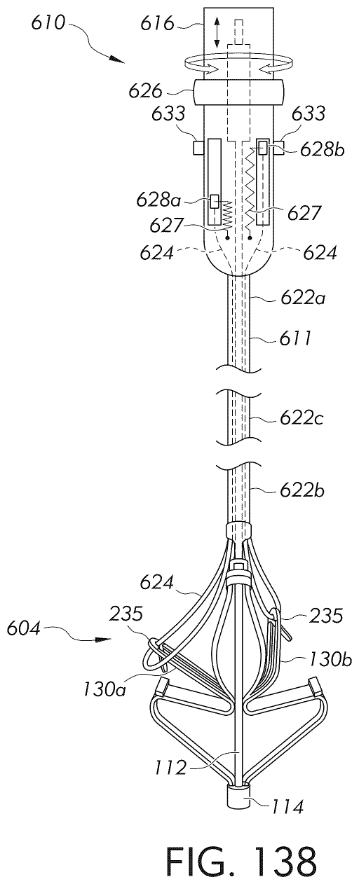

FIG. 138 illustrates an implementation of a device/implant catheter assembly 610 (e.g., a control catheter assembly, a device control catheter assembly, an implant control catheter assembly, an implant delivery catheter assembly, etc.). In the example illustrated by FIG. 138, the device/implant catheter assembly 610 can comprise an inner or actuation element 112, a coupler or capture mechanism 620, an outer shaft 611, a handle 616 (shown schematically), and clasp actuation lines 624. A proximal end portion 622a of the outer shaft 611 can be coupled to extend distally from the handle 616, and a distal end portion 622b of the outer shaft 611 can be coupled to the coupler or capture mechanism 620. The actuation element 112 can extend distally from the actuation control 626 (shown schematically in FIG. 138), through the handle 616, through the outer shaft 611, and through the coupler or capture mechanism 620. The actuation element 112 can be movable (e.g., axially and/or rotationally) relative to the outer shaft 611 and the handle 616. The clasp actuation lines 624 can extend through and be axially movable relative to the handle 616 and the outer shaft 611. The clasp actuation lines 624 can also be axially movable relative to the actuation element 112.

As shown in FIG. 138, the actuation element 112 (e.g., actuation rod, actuation tube, actuation shaft, actuation wire, etc.) of the device/implant catheter assembly 610 can be releasably coupled to the cap 114 of the device 604. The actuation element 112 extends from a proximal end portion 112a to a distal end portion 112b. In some implementations, the distal end portion 112b of the actuation element 112 can comprise external threads configured to releasably engage interior threads of the cap 114 of the device 604. As such, rotating the actuation element 112 in a first direction (e.g., clockwise) relative to the cap 114 of the device 604 releasably secures the actuation element 112 to the cap 114, while rotating the actuation element 112 in a second direction (e.g., counterclockwise) relative to the cap 114 of the device 604 releases the actuation element 112 from the cap 114.

In the example of FIG. 138, the outer shaft 611 of the device/implant catheter assembly 610 is an elongate shaft extending axially between the proximal end portion 622a, which is coupled to the handle 616, and the distal end portion 622b, which is coupled to the coupler 620. The outer shaft 611 can also include an intermediate portion 622c disposed between the proximal and distal end portions 622a, 622b. The outer shaft 611 can be formed from various materials, including metals and polymers. For example, in one particular implementation, the outer shaft can comprise stainless steel, polyether block amide (PEBA) and/or an outer covering or coating, such as a polymer that is reflowed over outer portions.

As shown in FIG. 138, the clasp actuation lines 624 are coupled to the clasps 130a, 130b on the device 604 through holes 235 in the clasps 130a, 130b and extend axially through the outer shaft 611 between the clasps 130a, 130b and the handle 616. In some implementations, the clasp actuation lines 624 are each coupled to a clasp control member 628a, 628b at the proximal end of the clasp actuation lines 624. Each clasp control member 628a, 628b can be, for example, an axially moving control or slider coupled to a corresponding clasp actuation line 624 to axially move the clasp actuation line 624 relative to the outer shaft 611 and the actuation element 112. Each of the clasp control members 628a, 628b can be operated independently of the other clasp control member such that each clasp actuation line 624 is moved relative to the outer shaft 611, the actuation element 112, and the other clasp actuation line 624, or the clasp control members 628a, 628b can be fixed with respect to one another (e.g., locked) such that the clasp actuation lines 624 are axially moved together relative to the outer shaft 611 and the actuation element 112.

In the illustrated example of FIG. 138, each clasp control member 628a, 628b is movable between a first position, as shown by clasp control member 628b in FIG. 138, and a second position, as shown by clasp control member 628a in FIG. 138. The first position of each clasp control member 628a, 628b is associated with an open position of the corresponding clasp 130 and the second position is associate with a closed position of the corresponding clasp 130. For example, in the illustrated example, moving one of the clasp control members 628a, 628b to the first position pulls the corresponding clasp 130 to the open position and moving one of the clasp control members 628a, 628b to the second position releases the corresponding clasp 130 to allow the clasp to move to a closed position.

In the illustrated example of FIG. 138, each clasp control member 628a, 628b is biased to the second position. Each clasp control member 628a, 628b can be biased to the second position in a variety of ways, such as springs, magnets, and elastic materials. In the illustrated example of FIG. 138, each clasp control member 628a, 628b is spring-loaded. In particular, the handle 616 includes a pair of springs 627. Each spring 627 is coupled to a corresponding one of the clasp control member 628a, 628b in such a way to bias the clasp control members 628a, 628b to the second position. Each of the clasp control members 628a, 628b can further have a releasable retaining device 633, such as for example, a locking mechanism, associated with each clasp control members 628a, 628b. The releasable retaining devices 633 can be configured in any suitable manner capable of holding the corresponding clasp control member 628a, 628b in the first position against the bias of the spring 627 until being released. Each of the retaining device 633 can include a manual interface for a user to actuate in order to release the clasp control member 628a, 628b and allow the spring 627 to move the clasp control member 628a, 628b to the second position.

The clasp control member 628a, 628b can be biased to the second position with sufficient force to rapidly move the clasp control member 628a, 628b from the first position to the second position to rapidly close the clasps. In some implementations, the clasp control member 628a, 628b moves from the first position to the second position in less than 500 milliseconds (ms), less than 100 ms, less than 75 ms, or less than 50 ms.

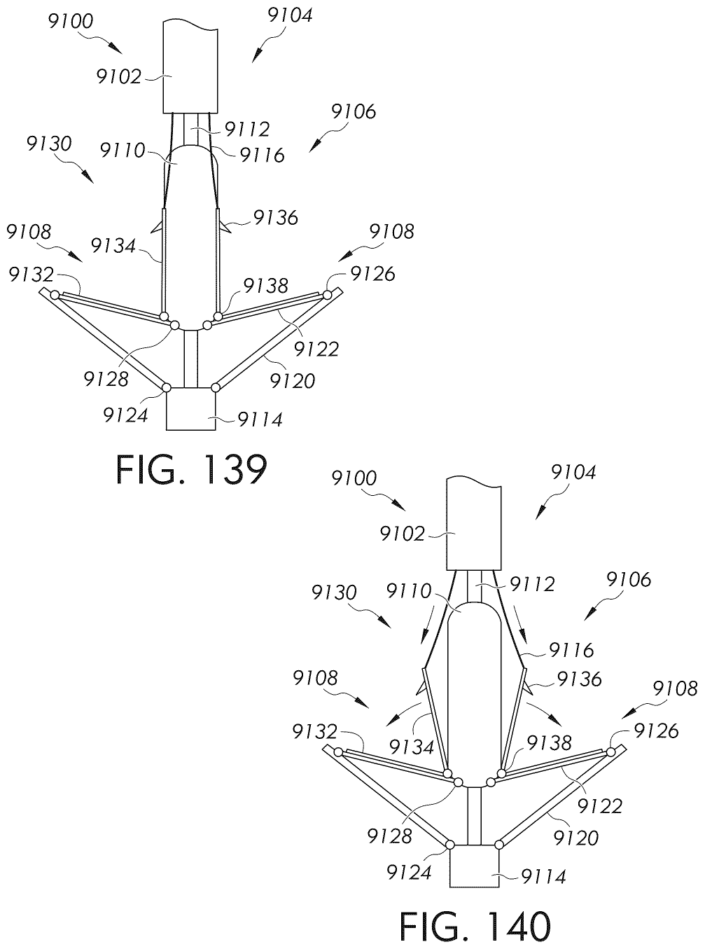

Referring now to FIG. 139, a schematically illustrated device or implant 9100 (e.g., an implantable prosthetic device, a prosthetic spacer device, a valve repair device, a treatment device, etc.) is shown in a partially open, grasp-ready condition. The device or implant 9100 corresponds to the previously described device 100 of FIGS. 8-14 and the description of the device 100 applies equally to the device 9100. However, the device 9100 can be any of the valve repair devices or treatment devices disclosed herein. The device or implant 9100 is deployed from a delivery system 9102. The delivery system 9102 can comprise one or more of a catheter, a sheath, a guide catheter/sheath, a delivery catheter/sheath, a steerable catheter, a device/implant catheter, a tube, a channel, a pathway, combinations of these, etc. The device or implant 9100 includes an optional coaptation portion/coaptation region 9104 and an anchor portion/anchor region 9106.

In some implementations, the coaptation portion 9104 of the device or implant 9100 includes a coaptation element 9110 (e.g., a spacer, coaption element, plug, membrane, sheet, etc.) that is adapted to be implanted between leaflets of a native valve (e.g., a native mitral valve, native tricuspid valve, etc.) and is slidable relative to an actuation element 9112 (e.g., actuation wire, actuation shaft, actuation tube, etc.).

The anchor portion 9106 includes one or more anchors 9108 that are actuatable between open and closed conditions and can take a wide variety of forms, such as, for example, paddles, gripping elements, or the like. Actuation of the actuation element 9112 opens and closes the anchor portion 9106 of the device 9100. The actuation element 9112 (as well as other means for actuating and actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 9106 relative to the coaptation portion 9104. Or, the actuation element can be unthreaded, such that pushing or pulling the actuation element 9112 moves the anchor portion 9106 relative to the coaptation portion 9104.

The anchor portion 9106 and/or anchors of the device can take a variety of different forms. For example, the anchor portion 9106 can have any of the features of any of the anchor portions disclosed herein. In the illustrated example, the anchor portion 9106 and/or anchors of the device 9100 include outer paddles 9120 and inner paddles 9122 that are, in some implementations, connected between a cap 9114 and coaptation element 9110 by portions 9124, 9126, 9128. The portions 9124, 9126, 9128 can be jointed and/or flexible to move between all of the positions described below. The interconnection of the outer paddles 9120, the inner paddles 9122, the coaptation element 9110, and the cap 9114 by the portions 9124, 9126, and 9128 can constrain the device to the positions and movements illustrated herein.

In some implementations, the delivery system 9102 includes a steerable catheter, device/implant catheter, and the actuation element 9112 (e.g., actuation wire, actuation shaft, etc.). These can be configured to extend through a guide catheter/sheath (e.g., a transseptal sheath, etc.). In some implementations, the actuation element 9112 extends through a delivery catheter and to or through the coaptation element 9110. Extending and retracting the actuation element 9112 increases and decreases the spacing between the coaptation element 9110 and the distal end of the device (e.g., the cap 9114 or other attachment portion), respectively. In some implementations, a collar or other attachment element removably attaches the coaptation element 9110 to the delivery system 9102, either directly or indirectly, so that the actuation element 9112 slides through the collar or other attachment element and, in some implementations, through a coaptation element 9110 during actuation to open and close the paddles 9120, 9122 of the anchor portion 9106 and/or anchors 9108.

In some implementations, the anchor portion 9106 and/or anchors 9108 can include attachment portions or gripping members. The gripping members can take a variety of different forms. For example, the gripping members can have any of the features of any of the gripping members disclosed herein. The illustrated gripping members can comprise clasps 9130 that include a base or fixed arm 9132, a moveable arm 9134, optional friction-enhancing elements or other securing structures 9136 (e.g., barbs, protrusions, ridges, grooves, textured surfaces, adhesive, etc.), and a joint portion 9138. The fixed arms 9132 are attached to the inner paddles 9122. In some implementations, the fixed arms 9132 are attached to the inner paddles 9122 with the joint portion 9138 disposed proximate the coaptation element 9110. The joint portion 9138 provides a spring force between the fixed and moveable arms 9132, 9134 of the clasp 9130. The joint portion 9138 can be any suitable joint, such as a flexible joint, a spring joint, a pivot joint, or the like. In some implementations, the joint portion 9138 is a flexible piece of material integrally formed with the fixed and moveable arms 9132, 9134. The fixed arms 9132 are attached to the inner paddles 9122 and remain stationary or substantially stationary relative to the inner paddles 9122 when the moveable arms 9134 are opened to open the clasps 9130 and expose the optional barbs, friction-enhancing elements, or securing structures 9136.

In some implementations, the clasps 9130 are opened by applying tension to actuation lines 9116 attached to the moveable arms 9134, thereby causing the moveable arms 9134 to articulate, flex, or pivot on the joint portions 9138. The actuation lines 9116 extend through the delivery system 9102 (e.g., through a steerable catheter and/or a device/implant catheter). Other actuation mechanisms are also possible.

The actuation lines 9116 can take a wide variety of forms, such as, for example, a line, a suture, a wire, a rod, a catheter, or the like. The clasps 9130 can be spring loaded so that in the closed position the clasps 9130 continue to provide a pinching force on the grasped native leaflet. Optional barbs, friction-enhancing elements, or securing structures 9136 of the clasps 9130 can grab, pinch, and/or pierce the native leaflets to further secure the native leaflets.

During implantation, the paddles 9120, 9122 can be opened and closed, for example, to press the native leaflets (e.g., native mitral valve leaflets, etc.) between the paddles 9120, 9122 and/or between the paddles 9120, 9122 and the coaptation element 9110 (e.g., a spacer, plug, membrane, gap filler, etc.). The clasps 9130 can be used to grasp and/or further secure the native leaflets by engaging the leaflets with optional barbs, friction-enhancing elements, or securing structures 9136 and pinching the leaflets between the fixed and moveable arms 9132, 9134. The optional barbs or other friction-enhancing elements 9136 (e.g., protrusions, ridges, grooves, textured surfaces, adhesive, etc.) of the clasps 9130 increase friction with the leaflets or can partially or completely puncture the leaflets. The actuation lines 9116 can be actuated separately so that each clasp 9130 can be opened and closed separately. Separate operation allows one leaflet to be grasped at a time, or for the repositioning of a clasp 9130 on a leaflet that was insufficiently grasped, without altering a successful grasp on the other leaflet. The clasps 9130 can be opened and closed relative to the position of the inner paddle 9122 (if the inner paddle is in an open or at least partially open position), thereby allowing leaflets to be grasped in a variety of positions as the particular situation requires.

In FIG. 139, the device 9100 is shown in a partially open, grasp-ready condition with the clasps 9130 in a fully open position such that the moveable arms 9134 of the clasps 9130 are in contact with an outer surface 9135 of the coaptation element 9110. Referring to FIG. 140, the device 9100 is shown in the partially open, grasp-ready condition but the clasps 9130 are in a drooped position. The drooped position is an undesired clasp position that can occur in some devices. In the drooped position, the clasps 9130 are actuated to be in the fully open position shown in FIG. 139, but instead of being in, or remaining in, the fully open position, the moveable arms 9134 are positioned, or prematurely move, away from the outer surface of the coaptation element 9110 (e.g., the angle between the fixed arm 9132 and the movable arm 9134 is less in the drooped position than in the fully open position). In the drooped position, there can be slack in the actuation lines 9116 such that the actuation lines 9116 do not hold the clasps 9130 in the fully open position. The clasps 9130 can be positioned at, or prematurely lower to, the drooped position if, for example, the actuation element 9112 stretches during implant elongation, the implant elongation length setting procedure is incorrect, or the length of the actuation lines 9116 is incorrect.

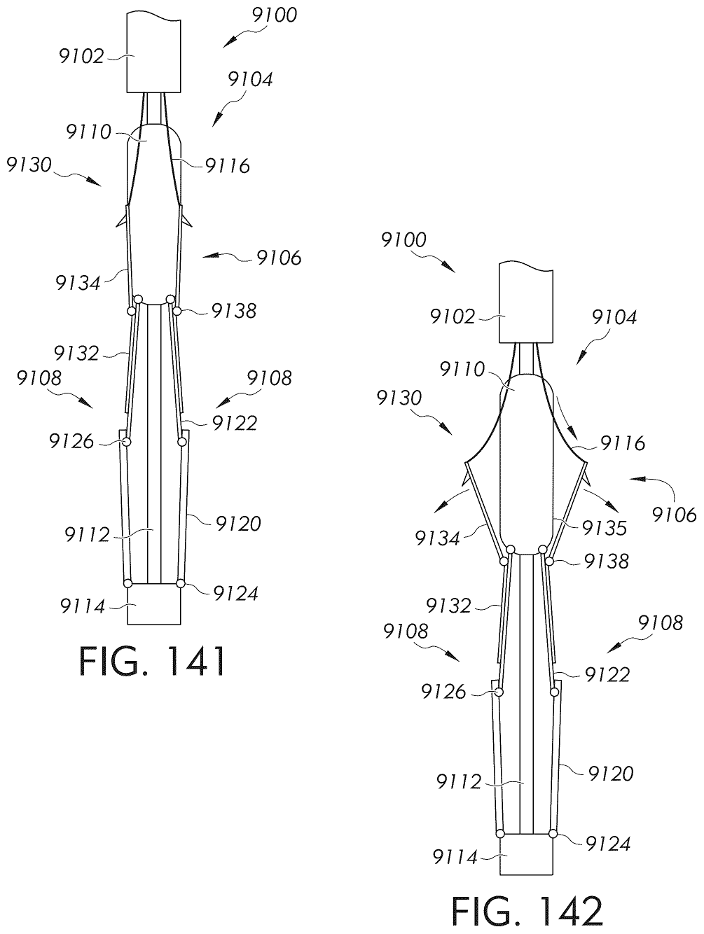

Referring to FIG. 141, the device 9100 is shown in an elongated condition with the clasps 9130 in a fully open position (e.g., approximately 180 degrees between fixed and moveable arms 9132, 9134 of the clasps 9130). FIG. 142 illustrates that the clasps 9130 can be in a drooped position when the device 9100 is also in the elongated condition. As shown in FIG. 142, instead of being in the fully open position, the moveable arms 9134 are positioned or move away from the coaptation element 9110 (e.g., the angle between the fixed arm 9132 and the movable arm 9134 is less in the drooped position than in the fully open position).

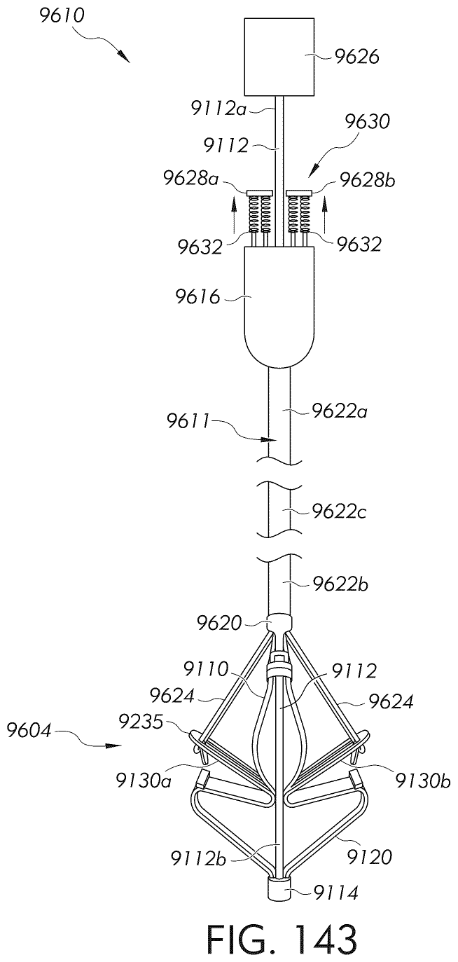

FIG. 143 is a schematic illustration of an example device/implant catheter assembly 9610 (e.g., a control catheter assembly, a device control catheter assembly, an implant control catheter assembly, an implant delivery catheter assembly, etc.) configured to compensate for clasp droop. The device/implant catheter assembly can take a variety of different forms. For example, the device/implant catheter assembly can have any of the features of any of the device/implant catheter assemblies disclosed herein. In the illustrated example, the device/implant catheter assembly 9610 corresponds to the previously disclosed device/implant catheter assembly 610 and the description of the device/implant catheter assembly 9610 applies equally to the device/implant catheter assembly 9610. The device/implant catheter assembly 9610 can include any feature for a device/implant catheter assembly 9610 discussed in the present application or the applications cited above.

In the example illustrated by FIG. 142, the device/implant catheter assembly 9610 can comprise an inner or actuation element 9112, a coupler or capture mechanism 9620, an outer shaft 9611, a handle 9616 (shown schematically), and clasp actuation elements 9624 (e.g., clasp actuation line, clasp actuation wire, clasp actuation shaft, clasp actuation rod, clasp actuation tube, etc.). A proximal end portion 9622a of the outer shaft 9611 can be coupled to extend distally from the handle 9616, and a distal end portion 9622b of the outer shaft 9611 can be coupled to the coupler or capture mechanism 9620. The actuation element 9112 can extend distally from an actuation control 9626 (shown schematically in FIG. 143), through the handle 9616, through the outer shaft 9611, and through the coupler or capture mechanism 9620. The actuation element 9112 can be movable (e.g., axially and/or rotationally) relative to the outer shaft 9611 and the handle 9616. The clasp actuation elements 9624 can extend through and be axially movable relative to the handle 9616 and the outer shaft 9611. The clasp actuation elements 9624 can also be axially movable relative to the actuation element 9112.

As shown in FIG. 143, the actuation element 9112 (e.g., actuation rod, actuation tube, actuation shaft, actuation wire, etc.) of the device/implant catheter assembly 9610 can be releasably coupled to the cap 9114 of the device 9604, either directly or through one or more intermediate components. The actuation element 9112 extends from a proximal end portion 9112a to a distal end portion 9112b. In some implementations, the distal end portion 9112b of the actuation element 9112 can comprise external threads configured to releasably engage interior threads of the cap 9114 or an intermediate component of the device 9604. As such, rotating the actuation element 9112 in a first direction (e.g., clockwise) relative to the cap 9114 of the device 9604 releasably secures the actuation element 9112 to the cap 9114 or intermediate component, while rotating the actuation element 9112 in a second direction (e.g., counterclockwise) relative to the cap 9114 of the device 9604 releases the actuation element 9112 from the cap 9114 or intermediate component. However, the actuation element 9112 can be coupled to the cap or intermediate component in a wide variety of different ways. For example, the actuation element can be coupled to the cap or intermediate component in any manner that any of the actuation elements disclosed in this application are coupled (see, for example, FIGS. 67-71).

In the example of FIG. 143, the outer shaft 9611 of the device/implant catheter assembly 9610 is an elongate shaft extending axially between the proximal end portion 9622a, which is coupled to the handle 9616, and the distal end portion 9622b, which is coupled to the coupler 9620. The outer shaft 9611 can also include an intermediate portion 9622c disposed between the proximal and distal end portions 9622a, 9622b. The outer shaft 9611 can be formed from various materials, including metals and polymers. For example, in one implementation, the outer shaft can comprise stainless steel, polyether block amide (PEBA) and/or an outer covering or coating, such as a polymer that is reflowed over outer portions.

As shown in FIG. 143, the clasp actuation elements 9624 are coupled to the clasps 9130a, 9130b on the device 9604 through holes 9235 in the clasps 9130a, 9130b and extend axially through the outer shaft 9611 between the clasps 9130a, 9130b and the handle 9616. In some implementations, the clasp actuation elements 9624 are each coupled to a clasp control member 9628a, 9628b at the proximal end of the clasp actuation elements 9624. Each clasp control member 9628a, 9628b can be, for example, an axially moving control or slider coupled to a corresponding clasp actuation element 9624 to axially move the clasp actuation element 9624 relative to the outer shaft 9611 and the actuation element 9112. Each of the clasp control members 9628a, 9628b can be operated independently of the other clasp control member such that each clasp actuation element 9624 is moved relative to the outer shaft 9611, the actuation element 9112, and the other clasp actuation element 9624, or the clasp control members 9628a, 9628b can be fixed with respect to one another (e.g., locked) such that the clasp actuation elements 9624 are axially moved together relative to the outer shaft 9611 and the actuation element 9112.

In the illustrated example of FIG. 143, each clasp control member 9628a, 9628b is moveable proximally to a first position to pull the corresponding clasp 9130a, 9130b to the open position and distally to a second position to release the corresponding clasp 9130a, 9130b to allow the clasp to move to a closed position. In the illustrated example of FIG. 143, the handle 9616 includes a droop compensation mechanism 9630 configured to prevent or reduce drooping of the clasps 9130a, 9130b. The droop compensation mechanism 9630 can prevent or reduce clasp droop in a variety of ways.

In the illustrated example of FIG. 143, the droop compensation mechanism 9630 includes one or more biasing elements 9632 configured to apply a force to each of the clasp actuation elements 9624, to each of the clasp control members 9628a, 9628b, or to both, such that the clasp actuation elements 9624 move the clasps 9130a, 9130b to the fully open position. For example, in some implementations, the one or more biasing elements 9632 can engage each of the clasp actuation elements 9624 and/or clasp control member 9628a, 9628b to cause each of the clasp actuation elements 9624 to pull the clasps 9130a, 9130b from the drooped position to the fully open position.

In some implementations, the one or more biasing elements 9632 are configured to remove slack from the clasp actuation elements 9624 (e.g., when the clasp actuation elements 9624 are configured as suture lines). The one or more biasing elements 9632 can be configured to apply the force during movement of the clasp control members 9628a, 9628b from the second position to the first position and after the clasp control members 9628a, 9628b are in the fully proximal first position. Thus, even when the clasp control members 9628a, 9628b have reached the fully proximal first position (e.g., the clasp control members cannot further pull on the clasp actuation elements 9624 to move the clasps 9630), the one or more biasing elements 9632 continue to apply a force to pull the first clasp toward the open position. For example, the one or more biasing elements 9632 can be configured to take-up (e.g., elastically stretch and return to its original length) a distance that is greater than or equal to an anticipated maximum clasp droop. For example, the one or more biasing elements 9632 can be configured to take-up 0.1 mm to 10 mm of droop, such as 0.2-5 mm of droop, such as 0.3-2 mm of droop, or any subrange of these ranges.

The one or more biasing elements 9632 can be configured in a variety of ways. In some examples, the one or more biasing elements 9632 can be springs, elastic elements capable of applying a suitable force to the clasp actuation elements 9624 and/or clasp control member 9628a, 9628b, elastic portion(s) of the clasp actuation elements 9624, clasp actuation elements 9624 made entirely of an elastic material, a clasp control member 9628a and/or 9628b having a spring or elastic element, etc. In some examples, the droop compensation mechanism 9630 can correct droop in one clasp 9130a independent of the other clasp 9130b (e.g., can move the clasp actuation element and/or clasp control member associated with one clasp independently from the clasp actuation control and/or clasp actuation element associated with the other clasp).

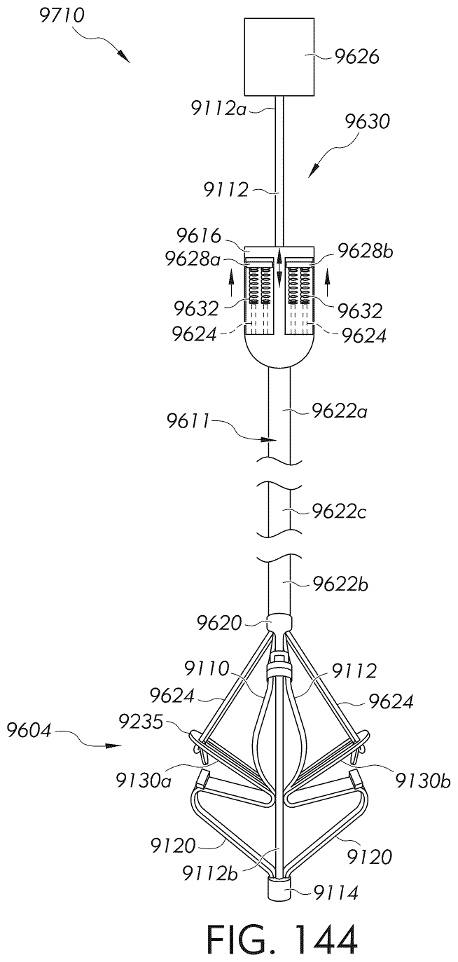

FIG. 144 is a schematic illustration of an example device/implant catheter assembly 9710 (e.g., a control catheter assembly, a device control catheter assembly, an implant control catheter assembly, an implant delivery catheter assembly, etc.) configured to compensate for clasp droop. The device/implant catheter assembly 9710 corresponds to the previously disclosed device/implant catheter assembly 9610 and the description of the device/implant catheter assembly 9610 applies equally to the device/implant catheter assembly 9710 with reference numbers of like elements kept the same. The device/implant catheter assembly 9710 can include any feature of any device/implant catheter assembly discussed in the present application or the applications cited above. In the example illustrated by FIG. 144, the droop compensation mechanism 9630 incorporates the one or more biasing elements 9632 as part of each of the clasp actuation elements 9624.

In some examples, each clasp actuation element 9624 can be made to be elastic, along the entire clasp actuation element 9624 or along one or more sections of the clasp actuation element 9624. For example, an entire elastic clasp actuation line or an elastic portion of a clasp actuation line can be configured to take-up (e.g., elastically stretch and return to its original length) a distance that is greater than or equal to an anticipated maximum clasp droop. For example, an entire elastic clasp actuation line or an elastic portion of a clasp actuation line can be configured to take-up 0.1 mm to 10 mm of droop, such as 0.2-5 mm of droop, such as 0.3 mm-2 mm of droop, or any subrange of these ranges. The elastic clasp actuation element 9624, or portions thereof, can function as the one or more biasing elements 9632. For example, in the example illustrated by FIG. 144, each clasp actuation element 9624 can include a portion near or adjacent each corresponding clasp control member 9628a, 9628b within the handle 9616 that functions as a biasing element 9632. The elastic portion or portions of the clasp actuation element 9624 can, however, be located along the clasp actuation element 9624 at any suitable location.

The clasp actuation element 9624, when configured as a suture line in some examples, can be made as a braided suture line. To increase the elasticity of the braided suture line or a portion of the suture line, the suture line or elastic portion of the suture line can be made with higher picks per inch of current suture material or from elastic materials. In some examples, one or more biasing elements 9632 can be separate elements from the clasp actuation element 9624 but attached inline. For example, the biasing elements 9632 can be springs or other suitable elastic elements that are separate from, but attached in-line with, the clasp actuation element 9624. The biasing elements 9632 can attach in-line with the clasp actuation element 9624 in any suitable manner. For example, a spring or elastic element can have suture lines attached to either end of the spring or elastic element with the combined spring/elastic element and suture lines functioning as the clasp actuation element 9624.

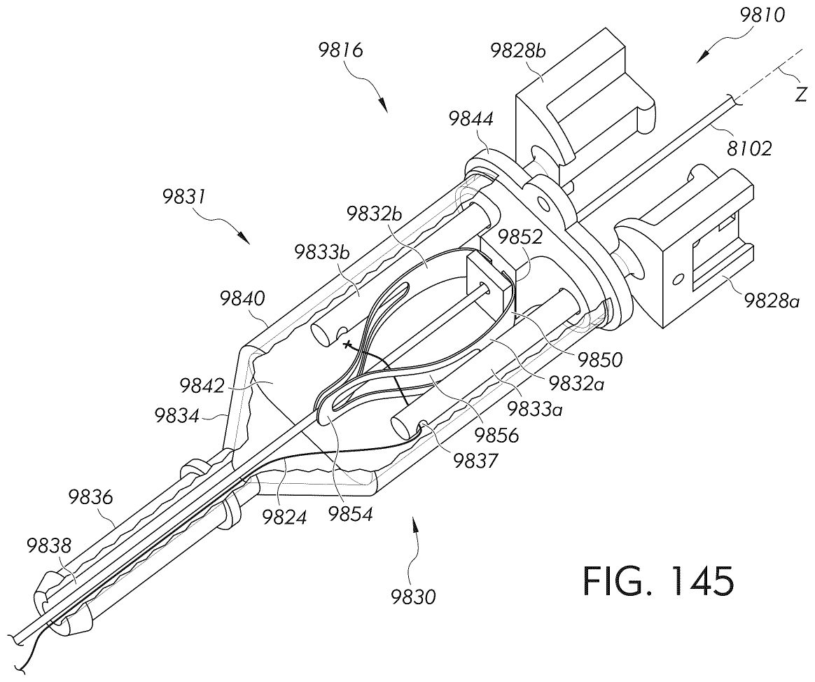

An example method of utilizing the elastic clasp actuation elements 9624 in the device/implant catheter assembly 9710 can include setting or selecting a length of the elastic clasp actuation elements 9624 when the device 9604 is in the fully elongated position, as shown in FIG. 141 and the clasp control members 9628a, 9628b are in a fully forward position, as shown in FIG. 145 (e.g., the clasps are in the first closed or fully forward position). This provides the minimum length of the clasp actuation element 9624 required to allow the clasps to move to the fully forward position at the fully elongated configuration. Thus, the biasing elements 9632 are in a non-stretched or natural condition so that little or no tension is applied to the clasps so that the clasps can move to the closed position without interference. When the clasp control members 9628a, 9628b are retracted to move the clasps up against the coaptation element 9110, the travel of the clasp control members can be configured to be greater than the travel when not using the biasing elements 9632. In this way, the biasing elements 9632 are stretched, which provides a pulling force on the clasps. As a result, if conditions that can result in clasp droop are present, the pulling force of the stretched biasing elements 9632 pulls the clasps to the fully open position.

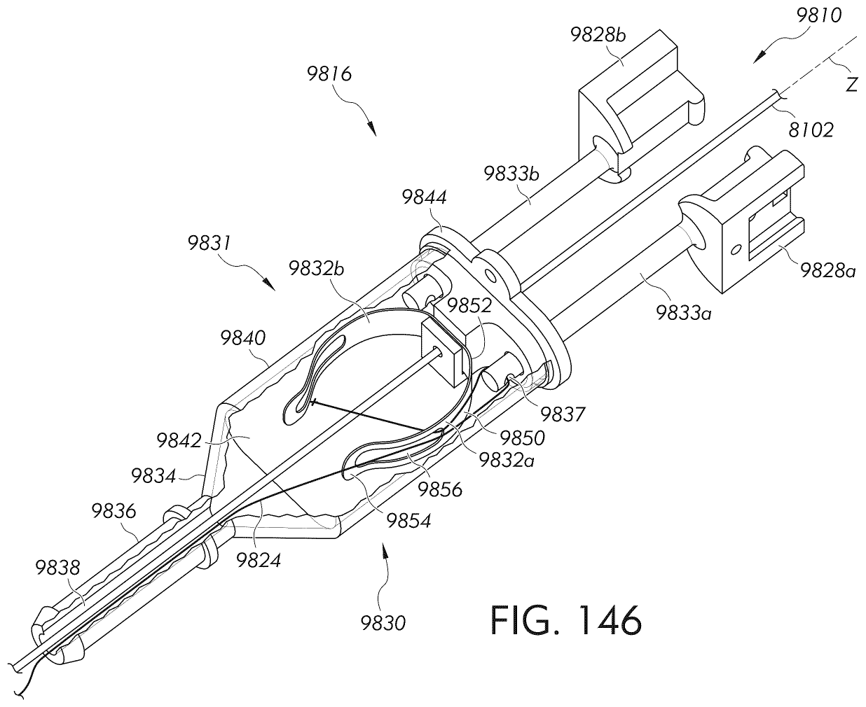

FIGS. 145-146 illustrate an example clasp actuation portion 9831 of a handle 9816 of a device/implant catheter assembly 9810 (e.g., a control catheter assembly, a device control catheter assembly, an implant control catheter assembly, an implant delivery catheter assembly, etc.). The clasp actuation portion 9831 corresponds to the previously disclosed clasp actuation portion 3631 of the handle 3616 and the description of the clasp actuation portion 3631 applies equally to the clasp actuation portion 9831. The clasp actuation portion 9831 can include any feature of any clasp actuation portion of any handle discussed in the present application or the applications cited above.

The clasp actuation portion 9831 can include first and second clasp control members 9828a, 9828b which are configured to move the clasps (e.g., clasps 9130 of device 9100) of the device via clasp actuation elements 9824. The clasps can be configured in the same manner as any of the clasps described herein. The clasp actuation portion 9831 can be configured in a variety of ways. In some implementations, the device/implant catheter assembly 9810 includes two clasp actuation elements 9824 each coupled to a corresponding clasp control member 9828a, 9828b at the proximal end of the clasp actuation elements 9824. For ease of illustration, only one clasp actuation element 9824 is shown in FIG. 145. In the illustrated example, the clasp actuation element 9824 is configured as a clasp actuation line. In some implementations, the clasp actuation element 9824 can be configured as a clasp actuation wire, a clasp actuation shaft, a clasp actuation rod, a clasp actuation tube, etc.

Each clasp control member 9828a, 9828b can be, for example, an axially-moving control or slider coupled to a corresponding clasp actuation element 9824 to axially move the clasp actuation element 9824 relative to the actuation element 8102. Each of the clasp control members 9828a, 9828b can be operated independently of the other clasp control member such that each clasp actuation element 9824 is moved relative to the actuation element 8102 and the other clasp actuation element 9824. The clasp control members 9828a, 9828b can also be fixed with respect to one another (e.g., locked) such that the clasp actuation elements 9824 are axially moved together relative to the actuation element 8102.

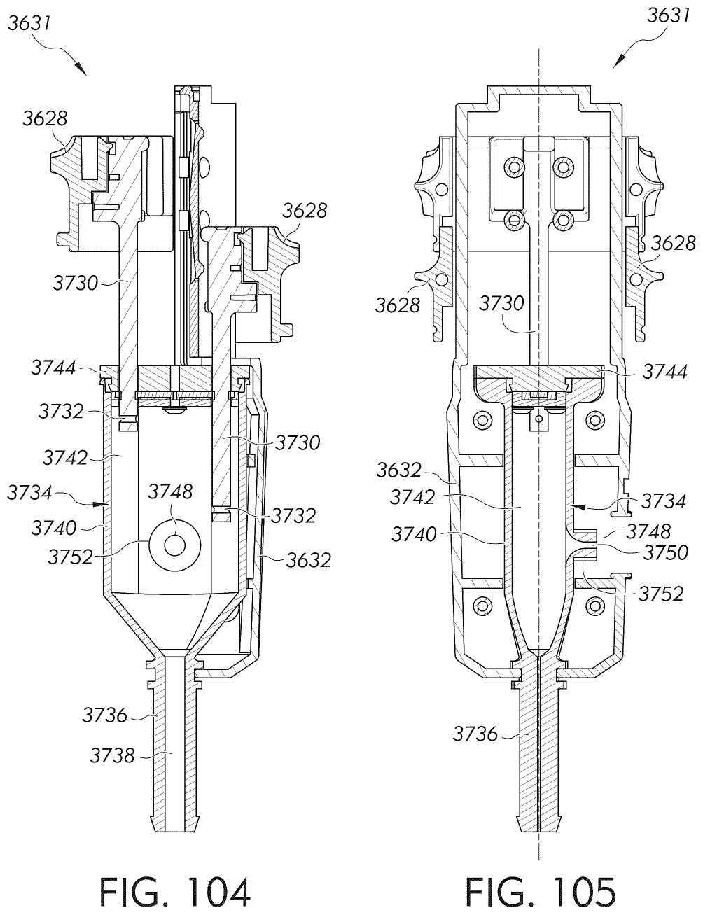

The clasp control members 9828a, 9828b can be configured in a variety of ways, including similar to any clasp control member disclosed herein. In the implementation depicted in FIGS. 145-146, the clasp control members 9828a, 9828b are similar to the clasp control members 3628 of FIGS. 104-105, and thus the previous description of the clasp control members 628 applies equally to the clasp control members 9828a, 9828b shown in FIGS. 145-146. The handle 9816 includes a first and second line-engaging member 9833a, 9833b configured to engage the clasp actuation elements 9824 to move the elements in response to movement of the clasp control members 9828a, 9828b. The line-engaging members 9833a, 9833b can be configured in a variety of ways. In the illustrated example, each line-engaging member 9833a, 9833b is configured as an elongated projection having a proximal end fixed to a corresponding clasp control member 9828a, 9828b and a distal end configured to engage a corresponding clasp actuation element 9824. In the illustrated example, the distal end of each line-engaging member 9833a, 9833b includes a passage 9837 through which a clasp actuation element 9824 can extend.

The handle 9816 includes a housing 9834 having a distal stem 9836 defining a passage 9838, a main body portion 9840 defining an interior space 9842, and a proximal cap 9844 through which the actuation element 8102 extends. In addition, each of the line-engaging members 9833a, 9833b extend distally from the respective clasp control members 9828a, 9828b through the proximal cap 9844 and into the interior space 9842. The main body portion 9840 further includes a line anchor (illustrated by an x in FIG. 145), similar to line anchor 3748 of FIGS. 104-105, to which the proximal ends of the clasp actuation elements 3624 attach.

In the example illustrated by FIG. 145-146, the clasp actuation portion 9831 includes a droop compensation mechanism 9830 configured to prevent or reduce drooping of the clasps of a device or implant. The droop compensation mechanism 9830 has features of the previously disclosed droop compensation mechanism 3630 of FIG. 144 and the description of the compensation mechanism 3630 applies to the droop compensation mechanism 9830. The droop compensation mechanism 9830 can include any feature of any droop compensation mechanism discussed in the present application.

In the illustrated example of FIGS. 145-146, the droop compensation mechanism 9830 includes a first biasing element 9832a and a second biasing element 9832b configured to apply a force to a corresponding clasp actuation element 9824 such that the clasp actuation elements 9624 move the clasps to the fully open position if conditions that can result in clasp droop are present. The biasing elements 9832a, 9832b can be configured in a variety of ways. In the illustrated example, the first biasing element 9832a is associated with the first clasp control member 9828a and the second biasing element 9832b is associated with the second clasp control member 9828b.

In the example illustrated by FIGS. 145 and 146, the first biasing element 9832a has an elongated body 9850 having a proximal end 9852 and a free distal end 9854. The proximal end 9852 is fixed relative to the housing 9834. For example, the proximal end 9852 can be fixedly attached to the proximal cap 9844 or another suitable portion of the handle 9816. The elongated body 9850 has an outward bowed or semi-elliptical shape resembling a band or leaf (e.g., a thin body with generally parallel inner and outer faces). A slot or opening 9856 can extend along a portion of the elongated body 9850 through which the corresponding clasp actuation element 9824 extends. In some examples, the slot or opening 9856 is positioned in a distal portion, such as a distal half, of the elongated body 9850. In some examples, the slot or opening 9856 extends along a majority of the distal half of the elongated body 9850.

The second biasing element 9832b can be identical to the first biasing element 9832a and arranged in mirror image to the first biasing element 9832a about a central longitudinal axis Z. Thus, the description of the first biasing element 9832a applies equally to the second biasing element 9832b. The biasing elements 9832a, 9832b are configured to be biased outward away from each other to a wide first position, as shown in FIG. 146.

Each clasp control member 9828a, 9828b is moveable between a first position (e.g., fully proximal), as shown in FIG. 146, and a second position (e.g., fully distal), as shown in FIG. 145. The first position of each clasp control member 9828a, 9828b is associated with an open position of the corresponding clasp (e.g., the clasp position shown in FIGS. 139 and 141) and the second position is associated with a closed position of the corresponding clasp (e.g., the clasp position shown in FIG. 8). For example, in the illustrated example, moving one of the clasp control members 9828a, 9828b to the first position pulls the corresponding clasp to the open position and moving one of the clasp control members 9828a, 9828b to the second position releases the corresponding clasp to allow the clasp to move to a closed position.

As shown in FIG. 145, when the clasp control members 9828a, 9828b are in the second position, each of the first and second line-engaging members 9833a, 9833b extends outward of and along a length of the respective first and second biasing elements 9832a, 9832b to engage and move the distal ends 9854 of the first and second biasing elements 9832a, 9832b toward each other such that the first and second biasing elements 9832a, 9832b are in the narrow second position, as shown in FIG. 145. In the narrow second position, the clasp actuation elements 9824 extend through the openings 9856 without the first and second biasing elements 9832a, 9832b applying any force onto the clasp actuation elements 9824. Thus, the clasps can close without disruption from any force from the first and second biasing elements 9832a, 9832b being applied to the clasp actuation elements 9824.

As shown in FIG. 146, when the clasp control members 9828a, 9828b are in the first position, the first and second line-engaging members 9833a, 9833b are retracted and do not engage the first and second biasing elements 9832a, 9832b. In addition, a portion of the clasp actuation elements 9824 are pulled by the first and second line-engaging members 9833a, 9833b to be along and outward of the first and second biasing elements 9832a, 9832b. With the clasp control members 9828a, 9828b retracted, the first and second biasing elements 9832a, 9832b are free to expand outward to the wide first position. When expanding outward toward the wide position, the first and second biasing elements 9832a, 9832b engage (e.g., an outer face of each biasing members can contact a corresponding clasp actuation elements) and apply a force to the clasp actuation elements 9824. The outward force applied to the clasp actuation elements 9824 by the first and second biasing elements 9832a, 9832b can take up any slack in the clasp actuation elements 9824 and cause the clasp actuation elements 9824 to pull the clasps to the fully open position, thus eliminating or reducing any droop in the clasp.

Depending on the amount of slack in the clasp actuation elements 9824, the first and second biasing elements 9832a, 9832b can expand to a position less than the wide first position (e.g., once all slack has been removed, the clasp actuation elements 9824 can prevent or inhibit the first and second biasing elements 9832a, 9832b from fully expanding). The first clasp control member 9828a and associated first biasing element 9832a can work independent of the second clasp control member 9828b and associated second biasing element 9832b. Thus, droop in one clasp can be addressed independent of droop in the other clasp. Further, if clasp droop is asymmetric (e.g., one clasp droops more than the other clasp), since each biasing element 9832a, 9832b is independent of the other, each biasing member can correct and compensate for the amount of droop present in the clasp associated with the biasing member.

### Examples

Example 1. A handle assembly for controlling a transvascular device, the handle assembly comprising: (i) a handle housing; (ii) a sheath extending distally from the handle housing; (iii) an actuation element extending through the sheath, the actuation element configured to be coupled to the device; and (iv) a paddle actuation control operatively coupled to the actuation element, wherein actuation of the paddle actuation control causes movement of the actuation element with respect to the handle housing and the sheath, wherein the movement of the actuation element causes the device to move between open and closed positions.

Example 2. The handle assembly according to example 1, further comprising a paddle width adjustment element extending through the actuation element, the paddle width adjustment element configured to be coupled to at least one of a pair of paddles of the device.

Example 3. The handle assembly according to example 2, further comprising a paddle width control operatively coupled to the paddle width adjustment element, wherein actuation of the paddle width control causes movement of the paddle width adjustment element with respect to the handle housing and the sheath, wherein the movement of the paddle width adjustment element causes a width of the at least one of the pair of paddles of the device to be moved from a first width to a second width.

Example 4. The handle assembly according to example 3, wherein the paddle actuation control is operatively coupled to a retractor to cause the movement of the actuation element and the paddle width control is operatively coupled to a drive member to cause the movement of the paddle width adjustment element, wherein actuation of the paddle actuation control causes movement of both the retractor and the drive member.

Example 5. The handle assembly according to example 4, further comprising a release control operatively coupled to the paddle width adjustment element, wherein actuation of the release control causes movement of the paddle width adjustment element and actuation element to decouple the device from the sheath.

Example 6. The handle assembly according to any one of examples 4-5, wherein the paddle actuation control is configured as a paddle actuation control knob, and wherein rotation of the paddle actuation control knob axially drives the retractor, and/or the paddle width control is configured as a paddle width control knob, and wherein rotation of the paddle width control knob axially drives the drive member.

Example 7. The handle assembly according to any one of examples 4-6, wherein the retractor is positioned within the handle distally from the drive member.

Example 8. The handle assembly according to any of claims 4-7, wherein the paddle actuation control is operatively coupled to a first internally threaded tube and actuation of the paddle actuation control rotates the first internally threaded tube to cause the movement of the retractor.

Example 9. The handle assembly according to any of examples 4-8, wherein the paddle width control is operatively coupled to a second internally threaded tube and actuation of the paddle width control rotates the second internally threaded tube to cause the movement of the drive member.

Example 10. The handle assembly according to example 9, wherein the handle assembly is configured such that movement of the retractor causes axial movement of the second internally threaded tube.

Example 11. The handle assembly according to any of examples 3-10, further comprising a release control operatively coupled to the paddle width adjustment element, wherein the release control is configured as a release control knob, and wherein rotation of the release control knob decouples the paddle width adjustment element from the paddle width control.

Example 12. The handle assembly according to example 11, further comprising a release nut that is operatively coupled with the paddle width control such that actuation of the paddle width control moves the release nut axially, and wherein rotation of the release control unscrews a release screw from the release nut to decouple the paddle width adjustment element from the paddle width control.

Example 13. The handle assembly according to example 11 or 12, wherein after the release control decouples the paddle width adjustment element from the paddle width control, the movement of the release control causes the movement of both the paddle actuation element and the paddle width adjustment element.

Example 14. A delivery system, comprising: (i) a steerable catheter assembly having a handle and a sheath extending from the handle in an axial direction, the sheath of the steerable catheter assembly having a distal end portion comprising a steerable section; (ii) a device/implant catheter assembly having a handle with a handle housing and a sheath that extends distally from the handle housing, wherein the sheath of the device/implant catheter assembly is extendable coaxially through the sheath of the steerable catheter assembly, wherein the handle of the device/implant catheter assembly comprises: (a) an actuation element extending through the sheath, the actuation element configured to be coupled to the device; and (b) a paddle actuation control operatively coupled to the actuation element, wherein actuation of the paddle actuation control causes movement of the actuation element with respect to the handle housing and the sheath, wherein the movement of the actuation element causes the device to move between open and closed positions.

Example 15. The delivery system according to example 14 further comprising a paddle width adjustment element extending through the actuation element, the paddle width adjustment element configured to be coupled to at least one of a pair of paddles of the device; and/or a paddle width control operatively coupled to the paddle width adjustment element, wherein actuation of the paddle width control causes movement of the paddle width adjustment element with respect to the handle housing and the sheath, wherein the movement of the paddle width adjustment element causes a width of the at least one of the pair of paddles of the device to be moved from a first width to a second width.

Example 16. The delivery system according to any one of examples 14-15 wherein the device/implant catheter assembly further comprises a release control operatively coupled to the paddle width adjustment element, wherein actuation of the release control causes movement of the paddle width adjustment element and actuation element to decouple the device from the sheath.

Example 17. The delivery system according to any one of examples 15-16 wherein the device/implant catheter assembly is configured such that actuation of the paddle width control and/or actuation of the paddle actuation control can cause axial and/or rotational movement of the release control.

Example 18. The delivery system according to any of examples 15-17, wherein the paddle actuation control is operatively coupled to a retractor to cause the movement of the actuation element and the paddle width control is operatively coupled to a drive member to cause the movement of the paddle width adjustment element, wherein actuation of the paddle actuation control can cause the movement of both the retractor and the drive member.

Example 19. The delivery system according to example 18, wherein the paddle actuation control is configured as a paddle actuation control knob, and wherein rotation of the paddle actuation control knob axially drives the retractor, and/or wherein the paddle width control is configured as a paddle width control knob, and wherein rotation of the paddle width control knob axially drives the drive member.

Example 20. The delivery system according to any of examples 18-19, wherein the retractor is positioned within the handle distally from the drive member.

Example 21. The delivery system according to any of examples 18-20, wherein the paddle actuation control is operatively coupled to a first internally threaded tube and actuation of the paddle actuation control rotates the first internally threaded tube to cause the axial and/or rotational movement of the retractor.

Example 22. The delivery system according to any of examples 18-21, wherein the paddle width control is operatively coupled to a second internally threaded tube and actuation of the paddle width control rotates the second internally threaded tube to cause the movement of the drive member.

Example 23. The delivery system according to example 22, wherein the device/implant catheter assembly is configured such that movement of the retractor causes axial movement of the second internally threaded tube.

Example 24. The delivery system according to any of examples 14-23, wherein the device/implant catheter assembly further comprises a release control operatively coupled to the paddle width adjustment element, wherein the release control is configured as a release control knob, and wherein rotation of the release control knob decouples the paddle width adjustment element from the paddle width control.

Example 25. The delivery system according to example 24, further comprising a release nut that is operatively coupled with the paddle width control such that actuation of the paddle width control moves the release nut axially, and wherein rotation of the release control unscrews a release screw from the release nut to decouple the paddle width adjustment element from the paddle width control.

Example 26. The delivery system according to example 24 or 25, wherein after the release control decouples the paddle width adjustment element from the paddle width control, axial movement of the release control moves both the paddle actuation element and the paddle width adjustment element.

Example 27. A method of delivering a device comprising: (i) obtaining the device coupled to an actuation element and a paddle width adjustment element extending from a distal end of a sheath of a device/implant catheter assembly, wherein the sheath is coupled at a proximal end of the sheath to a handle of the device/implant catheter assembly; (ii) advancing the sheath of the device/implant catheter assembly to position the device at a delivery site in an open configuration; (iii) actuating the paddle actuation control on the handle, thereby causing movement of the actuation element to move the device from the open configuration to a closed configuration; and (iv) actuating a paddle width control on the handle, thereby causing movement of the paddle width adjustment element to move a width of at least one of a pair of paddles from a first width to a second width.

Example 28. The method according to example 27, further comprising actuating a release control on the handle, thereby causing movement of the paddle width adjustment element and the actuation element to decouple the device from the sheath.

Example 29. The method according to example 28, wherein actuating of the paddle actuation control can cause axial movement of the release control and the paddle width adjustment element.

Example 30. The method according to any one of examples 27-29, wherein the paddle actuation control is a paddle actuation knob, and wherein actuating the paddle actuation control comprises rotating the paddle actuation knob such that one or more of the actuation element, the paddle width adjustment element, and the release control move axially relative to the housing of the handle.

Example 31. The method according to example 30, wherein the paddle width adjustment element is rotationally fixed during rotation of the paddle width control knob, and wherein the actuation element is rotationally fixed during rotation of the paddle actuation knob.

Example 32. The method according to any one of examples 28-30, wherein the release control is a release control knob, and wherein actuating the release control on the handle to decouple the device from the sheath further comprises rotating the release control knob to decouple the paddle width adjustment element from the paddle width control and then axially moving the release knob to axially move the actuation element and the paddle width adjustment element and the actuation element.

Example 33. A delivery system for a device having a plurality of clasps for securing native leaflets of a heart valve, the delivery system comprising: (i) a device/implant catheter assembly having a handle and a sheath extending from the handle in an axial direction, the sheath having a distal end portion comprising a capture mechanism for attaching the sheath to the device, the capture mechanism configured to move between a coupled position in which the capture mechanism is secured to the device and a release position in which the capture mechanism is decoupled from the device; and (ii) a first clasp actuation line configured to move a first clasp of the plurality of clasps between a closed position and an open position, the first clasp actuation line extending from the handle, through the sheath, and through a first aperture in the first clasp; and wherein a first distal end of the first clasp actuation line is attached to the capture mechanism when the capture mechanism is in the coupled position.

Example 34. The delivery system according to example 33, wherein the first distal end of the first clasp actuation line is captured between the capture mechanism and the device when the capture mechanism is in the coupled position.

Example 35. The delivery system according to example 33 or 34, wherein the capture mechanism includes a first longitudinally projecting finger and wherein the first distal end of the first clasp actuation line is attached to the first longitudinally projecting finger.

Example 36. The delivery system according to example 35, wherein the first distal end of the first clasp actuation line is formed as a first closed loop and the first longitudinally projecting finger extends through the first closed loop.

Example 37. The delivery system according to any of examples 33-36 wherein movement of the capture mechanism from the closed position to the open position and axial movement of the capture mechanism away from the device releases the first distal end of the first clasp actuation line from the capture mechanism.

Example 38. The delivery system according to any of examples 33-37, wherein the handle includes a first clasp control member that engages the first clasp actuation line and is movable relative to the housing.

Example 39. The delivery system according to example 37, wherein the first clasp control member includes a first passage through which the first clasp actuation line extends, wherein the first clasp actuation line slides through the first passage when the first clasp control member moves relative to the housing.

Example 40. The delivery system according to example 39, wherein the first clasp actuation line includes a first proximal end fixed relative to the to the handle.

Example 41. The delivery system according to any of examples 33-40, further comprising a second clasp actuation line configured to move a second clasp of the plurality of clasps between a closed position and an open position, the second clasp actuation line extending from the handle, through the sheath, and through a second aperture in the second clasp; and wherein a second distal end of the second clasp actuation line is attached to the capture mechanism when the capture mechanism is in the coupled position.

Example 42. The delivery system according to example 41, wherein the second distal end of the second clasp actuation line is captured between the capture mechanism and the device when the capture mechanism is in the coupled position.

Example 43. The delivery system according to example 41 or 42, wherein the capture mechanism includes a second longitudinally projecting finger and wherein the second distal end of the second clasp actuation line is attached to the second longitudinally projecting finger.

Example 44. The delivery system according to example 43, wherein the second distal end of the second clasp actuation line is formed as a second closed loop and the second longitudinally projecting finger extends through the second closed loop.

Example 45. The delivery system according to any of examples 41-44 wherein movement of the capture mechanism from the closed position to the open position and axial movement of the capture mechanism away from the device releases the second distal end of the second clasp actuation line from the capture mechanism.

Example 46. The delivery system according to any of examples 41-45, wherein the handle includes a second clasp control member that engages the second clasp actuation line and is movable relative to the housing.

Example 47. The delivery system according to example 46, wherein the second clasp control member slides axially relative to the handle.

Example 48. The delivery system according to example 46 or 47, wherein the second clasp control member includes a second passage through which the second clasp actuation line extends, wherein the second clasp actuation line slides through the second passage when the second clasp control member moves relative to the housing.

Example 49. The delivery system according to example 48, wherein the second clasp actuation line includes a second proximal end fixed relative to the to the handle.

Example 50. The delivery system according to any of examples 41-49, wherein the second clasp control member and the first clasp control member are movable independently of each other.

Example 51. A method of treating a heart valve with a treatment device or valve repair device, comprising: (i) closing a first clasp of the treatment device or valve repair device to grasp a first leaflet of the heart valve by releasing tension in a first clasp actuation line coupled to the first clasp; (ii) closing a second clasp of the valve repair device to grasp a second leaflet of the heart valve by releasing tension in a second clasp actuation line coupled to the second clasp; and (iii) releasing the treatment device or valve repair device from a capture mechanism and withdrawing the capture mechanism from the treatment device or valve repair device, wherein releasing the treatment device or valve repair device from the capture mechanism and withdrawing the capture mechanism from the valve repair device comprises uncoupling the first clasp actuation line from the first clasp and uncoupling the second clasp actuation line from both the second clasp.

Example 52. The method according to example 51, wherein the first clasp and the second clasp are simultaneously closed.

Example 53. The method according to example 51, wherein the first clasp and the second clasp are sequentially closed.

Example 54. The method according to any of examples 51-53, wherein connecting the first clasp actuation line to the capture mechanism comprises capturing a first distal end of the first clasp actuation line between the capture mechanism and the treatment device or valve repair device.

Example 55. The method according to example 54, wherein the first distal end includes a first closed loop and connecting the first clasp actuation line to the capture mechanism further comprises receiving a first portion of the capture mechanism through the first closed loop.

Example 56. The method according to example 55, wherein releasing the first clasp actuation line from the capture mechanism further comprises withdrawing the first portion from the first closed loop.

Example 57. The method according to example 55 or 56, wherein releasing the first clasp actuation line from the first clasp further comprises pulling the first closed loop through a first aperture in the first clasp.

Example 58. The method according to any of examples 51-57, wherein connecting the second clasp actuation line to the capture mechanism includes capturing a second distal end of the second clasp actuation line between the capture mechanism and the treatment device or valve repair device.

Example 59. The method according to example 58, wherein the second distal end includes a second closed loop and connecting the second clasp actuation line to the capture mechanism further comprises receiving a second portion of the capture mechanism through the second closed loop.

Example 60. The method according to example 59, wherein releasing the second clasp actuation line from the capture mechanism further comprises withdrawing the second portion from the second closed loop.

Example 61. The method according to example 59 or 60, wherein releasing the second clasp actuation line from the second clasp further comprises pulling the second closed loop through a second aperture in the second clasp.

Example 62. A handle assembly for controlling a transvascular device having one or more clasps for securing one or more portions of tissue, the handle assembly comprising: (i) a handle housing; (ii) a sheath extending distally from the handle housing; (iii) a first clasp actuation line extending through the sheath, the first clasp actuation line operatively coupled to a first clasp of the plurality of clasps on the device; and (iv) a first clasp control member operatively connected to the first clasp actuation line, the first clasp control member movable relative to the housing between a first position associated with the first clasp being in an open position and a second position associated with the clasp being in a closed position, and wherein the first clasp control member is biased to the second position.

Example 63. The handle assembly according to example 62, further comprising a first spring arranged to bias the first clasp control member to the second position.

Example 64. The handle assembly according to example 62 or 63, further comprising a first releasable retaining device configured to hold the first clasp control member in the first position.

Example 65. The handle assembly according to any of examples 62-64, wherein the first clasp control member is movable axially relative to the housing between the first position and the second position.

Example 66. The handle assembly according to any of example 63-65, wherein first clasp control member is biased to move from the first position to the second position in less than 500 milliseconds.

Example 67. The handle assembly according to any of example 63-65, wherein first clasp control member is biased to move from the first position to the second position in less than 75 milliseconds.

Example 68. The handle assembly according to any of examples 63-67, further comprising: (i) a second clasp actuation line extending through the sheath, the second clasp actuation line operatively coupled to a second clasp of the plurality of clasps on the device; and (ii) a second clasp control member operatively connected to the second clasp actuation line, the second clasp control member is movable relative to the housing between a third position associated with the second clasp being in an open position and a fourth position associated with the second clasp being in a closed position, and wherein the second clasp control member is biased to the fourth position.

Example 69. The handle assembly according to example 68, further comprising a second spring arranged to bias the second clasp control member to the fourth position.

Example 70. The handle assembly according to example 68 or 69, further comprising a second releasable retaining device configured to hold the second clasp control member in the third position.

Example 71. The handle assembly according to any of examples 68-70, wherein the second clasp control member is movable axially relative to the housing between the third position and the fourth position.

Example 72. The handle assembly according to any of examples 68-71, wherein second clasp control member is biased to move from the third position to the fourth position in less than 500 milliseconds.

Example 73. The handle assembly according to any of examples 68-71, wherein second clasp control member is biased to move from the third position to the fourth position in less than 75 milliseconds.

Example 74. The handle assembly according to any of examples 68-73, wherein the first clasp control member is movable from the first position to the second position independent of movement of the second clasp control member.

Example 75. A method of using a treatment device or valve repair device having a plurality of clasps for securing native leaflets of a heart valve, the method comprising: (i) delivering the treatment device or valve repair device to the heart valve via a device/implant catheter assembly having a first clasp actuation line that holds a first clasp of the treatment device or valve repair device in an open position and a second clasp actuation line that holds a second clasp of the treatment device or valve repair device in an open position; (ii) closing the first clasp of the treatment device or valve repair device to grasp a first leaflet of the heart valve by releasing tension in the first clasp actuation line; (iii) closing the second clasp of the treatment device or valve repair device to grasp a second leaflet of the heart valve by releasing tension in the second clasp actuation line; wherein releasing tension in the first clasp actuation line comprising biasing a first clasp control member from a first position to a second position; and wherein releasing tension in the second clasp actuation line comprising biasing a second clasp control member from a third position to a fourth position.

Example 76. The method according to example 75, wherein the first clasp control member is mounted on a handle of the device/implant catheter assembly and the first clasp control member moves axially relative to the handle when biased from the first position to a second position.

Example 77. The method according to example 75 or 76, wherein the first clasp control member is biased from the first position to the second position by a first spring.

Example 78. The method according to example 76 or 77, wherein the second clasp control member is mounted on the handle of the device/implant catheter assembly and the second clasp control member moves axially relative to the handle when biased from the third position to a fourth position.

Example 79. The method according to any of examples 75-78, wherein the second clasp control member is biased from the third position to the fourth position by a second spring.

Example 80. The method according to any of example 75-79, wherein the first clasp and the second clasp are simultaneously closed.

Example 81. The method according to any of examples 75-79, wherein the first clasp and the second clasp are sequentially closed.

Example 82. The method according to any of examples 75-81, wherein first clasp control member is biased to move from the first position to the second position in less than 500 milliseconds.

Example 83. The method according to any of examples 75-81, wherein first clasp control member is biased to move from the first position to the second position in less than 75 milliseconds.

Example 84. The method according to any of examples 75-83, wherein second clasp control member is biased to move from the third position to the fourth position in less than 500 milliseconds.

Example 85. The method according to any of examples 75-83, wherein second clasp control member is biased to move from the third position to the fourth position in less than 75 milliseconds.

Example 86. A clasp actuation line for actuating a clasp of a treatment device or repair device, comprising: a braided body having a proximal end, a distal end opposite the proximal end, and a closed loop formed at the distal end.

Example 87. The clasp actuation line according to example 86, wherein the braided body is formed from ultra-high-molecular-weight polyethylene material.

Example 88. The clasp actuation line according to example 86 or 87, wherein the braided body has 4 to 100 ends using 10 to 400 dtex yarn.

Example 89. The clasp actuation line according to any of examples 86-88, wherein the clasp actuation line has a tensile strength in the range of 20-100 N and a diameter in the range of 0.003 inches to 0.008 inches.

Example 90. The clasp actuation line according to any of examples 86-89, wherein the closed loop has a nominal loop diameter in the range or 0.0275 inches to 0.0425 inches and a circumference in the range of 0.07 inches to 0.15 inches.

Example 91. The clasp actuation line according to any of examples 86-90, wherein the closed loop is formed by a bifurcated braided portion of the braided body.

Example 92. The clasp actuation line according to any of example 86-91, wherein a distal terminal end of the braided body extends axially through a portion of the braided body at a location proximal the closed loop to form a tucked-in portion.

Example 93. The clasp actuation line according to example 92, wherein the tucked-in portion has a length of 0.25 inches or greater.

Example 94. The clasp actuation line according to example 92, wherein the tucked-in portion has a length of 1 inch or greater.

Example 95. The clasp actuation line according to any of example 86-91, wherein a distal terminal end portion of the braided body extends laterally through a portion of the braided body at a location proximal the closed loop to form a threaded portion.

Example 96. The clasp actuation line according to any of example 86-91, wherein a distal terminal end portion of the braided body threads laterally back and forth, at least twice, through a portion of the braided body at a location proximal the closed loop to form a threaded portion.

Example 97. The clasp actuation line according to example 95 or 96, wherein the distal terminal end portion of the braided body extends axially through a portion of the braided body at a location proximal the closed loop to form a tucked-in portion proximal the threaded portion.

Example 98. The clasp actuation line according to example 97, wherein the tucked-in portion has a length of 0.25 inches or greater.

Example 99. The clasp actuation line according to example 97, wherein the tucked-in portion has a length of 1 inch or greater.

Example 100. A method of forming a clasp actuation line for actuating a clasp of a treatment device or valve repair device, the method comprising: (i) braiding an elongated body; and (ii) forming a closed loop at a distal end of the elongated body.

Example 101. The method according to example 100, wherein braiding the elongated body includes braiding a bifurcated portion to form the closed loop.

Example 102. The method according to example 101, further comprising heat sealing a distal terminal end of the braided body.

Example 103. The method according to example 100, wherein braiding an elongated body further comprising braiding a plurality of a bifurcated portions separated by unitary portion, and wherein the method further comprises cutting the elongated body into sections comprising a single bifurcated portion adjacent a distal end of the section.

Example 104. The method according to example 100, wherein forming the closed loop comprises extending a distal terminal end of the braided body axially through a portion of the braided body to form a tucked-in portion proximal the closed loop.

Example 105. The method according to example 100, wherein forming the closed loop comprises extending a distal terminal end portion of the braided body laterally through a portion of the braided body to form a threaded portion at a location proximal the closed loop.

Example 106. The method according to example 105 further comprising extending the distal terminal end portion of the braided body clasp actuation line laterally through the portion of the braided body at least twice.

Example 107. The method according to example 105 or 106, further comprising extending a distal terminal end of the braided body axially through a portion of the braided body to form a tucked-in portion proximal the threaded portion.

Example 108. The method according to example 100, wherein forming the closed loop comprises extending a piercing device laterally through a portion adjacent a distal end of the braided body to form a lateral passage through the braided body.

Example 109. A handle assembly for controlling a transvascular device, the handle assembly comprising: (i) a handle housing; (ii) a sheath extending distally from the handle housing; (iii) an actuation element extending through the sheath, the actuation element configured to be coupled to the device; and (iv) a paddle actuation control operatively coupled to the actuation element, wherein actuation of the paddle actuation control causes movement of the actuation element with respect to the handle housing and the sheath, wherein movement of the actuation element causes the device to move between open and closed positions.

Example 110. The handle assembly according to example 109, further comprising a paddle width adjustment element extending through the actuation element, the paddle width adjustment element configured to be coupled to at least one of a pair of paddles of the device; and a paddle width control operatively coupled to the paddle width adjustment element, wherein actuation of the paddle width control causes movement of the paddle width adjustment element with respect to the handle housing and the sheath, wherein movement of the paddle width adjustment element causes a width of the at least one of the pair of paddles of the device to be moved from a first width to a second width.

Example 111. The handle assembly according to example 109 or example 110, wherein a distal end of the paddle actuation control comprises external threads configured to engage with internal threads of the handle housing.

Example 112. The handle assembly according to any one of examples 110-111, wherein the paddle width control is configured as a paddle width control knob, and wherein rotation of the paddle width control knob axially drives a frame that is attached to the actuation element.

Example 113. The handle assembly according to example 112, wherein the paddle width control knob is rotatable relative to the frame.

Example 114. The handle assembly according to example 112 or example 113, wherein the paddle actuation control is configured as a paddle actuation knob that is rotatable relative to the frame.

Example 115. The handle assembly according to any one of examples 112-114, wherein rotation of the paddle width control knob axially drives the paddle width control knob and the paddle width adjustment element with respect to the paddle actuation knob and the actuation element.

Example 116. A delivery system comprising: (i) a steerable catheter assembly having a handle and a sheath extending from the handle in an axial direction, the sheath of the steerable catheter assembly having a distal end portion comprising a steerable section; (ii) a device/implant catheter assembly having a handle and a sheath extendable coaxially through the sheath of the steerable catheter assembly, wherein the handle of the device/implant catheter assembly comprises: (a) a handle housing, wherein the sheath of the device/implant catheter assembly extends distally from the handle housing; (b) an actuation element extending through the sheath, the actuation element configured to be coupled to the device; and (c) a paddle actuation control operatively coupled to the actuation element, wherein actuation of the paddle actuation control causes movement of the actuation element with respect to the handle housing and the sheath, wherein the movement of the actuation element causes the device to be moved between an open configuration and a closed configuration.

Example 117. The device delivery system according to example 116, further comprising a paddle width adjustment element extending through the actuation element, the paddle width adjustment element configured to be coupled to at least one of a pair of paddles of the device; and a paddle width control coupled to the paddle width adjustment element, wherein actuation of the paddle width control causes movement of the paddle width adjustment element with respect to the handle housing and the sheath, wherein the movement of the paddle width adjustment element causes a width of the at least one of the pair of paddles of the device to be moved from a first width to a second width.

Example 118. The device delivery system according to example 116 or example 117, wherein a distal end of the paddle actuation control comprises external threads configured to engage with internal threads of the handle housing.

Example 119. The device delivery system according to any one of examples 117-118, wherein the paddle width control is configured as a paddle width control knob, and wherein rotation of the paddle width control knob axially drives a frame that is attached to the actuation element.

Example 120. The device delivery system according to example 119, wherein the paddle width control knob is rotatable relative to the frame.

Example 121. The handle assembly according to example 119 or example 120, wherein rotation of the paddle width control knob in a first direction axially drives the paddle width control knob and the paddle width adjustment element in a direction away from the paddle actuation control and the actuation element.

Example 122. A method of delivering a device comprising: (i) obtaining the device coupled to an actuation element and a paddle width adjustment element extending from a distal end of a sheath of a device/implant catheter assembly, wherein the sheath is coupled at a proximal end of the sheath to a handle of the device/implant catheter assembly; (ii) advancing the sheath of the device/implant catheter assembly to position the device at a delivery site; (iii) actuating a paddle actuation control on the handle, thereby causing movement of the actuation element to move the device from a closed configuration to an open configuration; (iv) actuating a paddle width control on the handle, thereby causing movement of the paddle width adjustment element to move a width of at least one of a pair of paddles from a first width to a second width; (v) actuating the paddle actuation control on the handle, thereby causing movement of the actuation element to move the device from the open configuration to a closed configuration; and (vi) decoupling the device from the actuation element and the paddle width adjustment element.

Example 123. The method according to example 122, wherein moving the paddle actuation control in a single direction can move the device from a fully elongated configuration to the open configuration and move the device from the open configuration to the closed configuration.

Example 124. The method according to example 122 or example 123, wherein the paddle width adjustment element is coupled to the at least one of the pair of paddles through an inner end, wherein axial movement of the paddle width adjustment element causes axial movement of the inner end with respect to an actuation portion of the device, and wherein axial movement of the inner end causes the at least one of the pair of paddles to move relative to the actuation portion of the device effective to move the width of at least one of the pair of paddles from the first width to the second width.

Example 125. The method according to any one of examples 122-124, wherein the paddle actuation control is a paddle actuation knob, and wherein actuating the paddle actuation control comprises rotating the paddle actuation knob such that the paddle actuation knob and the paddle width control move axially relative to the housing of the handle.

Example 126. The method according to any one of examples 122-125, wherein the paddle width control is a paddle width control knob, and wherein actuating the paddle width control comprises rotating the paddle width control knob such that the paddle width control knob and the paddle width adjustment element move axially relative to a frame coupled to the paddle actuation knob.

Example 127. The method according to example 126, wherein the paddle width adjustment element is rotationally fixed during rotation of the paddle width control knob, and wherein the actuation element is rotationally fixed during rotation of the paddle actuation knob.

Example 128. The method according to any one of examples 122-127, wherein decoupling the device comprises: (i) releasing a first end of the paddle width adjustment element and pulling a second end of the paddle width adjustment element to cause the first end of the paddle width adjustment element to be pulled through the sheath of the device/implant catheter assembly and the actuation element; and (ii) releasing a first end of the actuation element and pulling a second end of the actuation element to cause the first end of the actuation element to be pulled through the sheath of the device/implant catheter assembly.

Example 129. A handle assembly for controlling a transvascular device, the handle assembly comprising: (i) a handle housing; (ii) a sheath extending distally from the handle housing; (iii) an actuation element extending through the sheath, the actuation element configured to be coupled to the device; (iv) a paddle width adjustment element extending through the actuation element, the paddle width adjustment element configured to be coupled to at least one of a pair of paddles of the device; and (v) a paddle actuation control coupled to the actuation element, wherein actuation of the paddle actuation control causes movement of the actuation element with respect to the handle housing and the sheath, wherein the movement of the actuation element causes the device to be moved between an open configuration and a closed configuration.

Example 130. The handle assembly according to example 129, further comprising a paddle width control coupled to the paddle width adjustment element, wherein actuation of the paddle width control causes movement of the paddle width adjustment element with respect to the handle housing and the sheath, wherein the movement of the paddle width adjustment element causes a width of the at least one of the pair of paddles of the device to be moved from a first width to a second width.

Example 131. The handle assembly according to any one of examples 129-130, a pair of clasp actuation lines extending through the sheath, each clasp actuation line of the pair of clasp actuation lines configured to be coupled to the device; and a pair of clasp control members, wherein each clasp control member of the pair of clasp control members is movable relative to the handle housing, wherein the movement of each clasp control member causes a clasp of the device to be moved between an open configuration and a closed configuration.

Example 132. The handle assembly according to example 131, wherein each clasp actuation line of the pair of clasp actuation lines is coupled to a suture lock extending from a proximal end of the handle housing.

Example 133. The handle assembly according to any one of examples 129-131, wherein the paddle width control is coupled to a planetary gearbox and actuation of the paddle width control is effective to cause rotation of the planetary gearbox.

Example 134. The handle assembly according to example 133, wherein the planetary gearbox comprises an elongated central gear, wherein the elongated central gear is coupled to the paddle width adjustment element through a rotationally fixed follower such that rotation of the elongated central gear causes movement of the rotationally fixed follower, which in turn causes movement of the paddle width adjustment element with respect to the housing.

Example 135. The handle assembly according to example 134, wherein external teeth of the elongated central gear engage with teeth of the pair of planet gears.

Example 136. A delivery system comprising: (i) a steerable catheter assembly having a handle and a sheath extending from the handle in an axial direction, the sheath of the steerable catheter assembly having a distal end portion comprising a steerable section; (ii) a device/implant catheter assembly having a handle and a sheath extendable coaxially through the sheath of the steerable catheter assembly, wherein the device/implant catheter assembly comprises: (a) a handle housing, wherein the sheath of the device/implant catheter assembly extends distally from the handle housing; (b) an actuation element extending through the sheath, the actuation element configured to be coupled to the device; (c) a paddle width adjustment element extending through the actuation element, the paddle width adjustment element configured to be coupled to at least one of a pair of paddles of the device; (d) a paddle actuation control coupled to the actuation element, wherein actuation of the paddle actuation control causes movement of the actuation element with respect to the handle housing and the sheath, wherein the movement of the actuation element causes the device to be moved between an open configuration and a closed configuration; and (e) a paddle width control coupled to the paddle width adjustment element, wherein actuation of the paddle width control causes movement of the paddle width adjustment element with respect to the handle housing and the sheath, wherein the movement of the paddle width adjustment element causes a width of the at least one of the pair of paddles of the device to be moved from a first width to a second width.

Example 137. The device delivery system according to example 136, wherein the device/implant catheter assembly further comprises (i) a pair of clasp actuation lines extending through the sheath, each clasp actuation line of the pair of clasp actuation lines configured to be coupled to the device; and (ii) a pair of clasp control members, wherein each clasp control member of the pair of clasp control members is movable relative to the handle housing, wherein the movement of each clasp control member causes a respective clasp of the device to be moved between an open configuration and a closed configuration; and wherein each clasp actuation line of the pair of clasp actuation lines is coupled to a suture lock extending from a proximal end of the handle housing.

Example 138. The device delivery system according to any one of examples 136-137, wherein each suture lock is angled with respect to a central axis extending through the handle of the device/implant catheter assembly.

Example 139. The device delivery system according to any one of examples 136-138, wherein the paddle width control is configured as a paddle width control knob that is coupled to a planetary gearbox and rotation of the paddle width control knob is effective to cause rotation of the planetary gearbox.

Example 140. The device/implant catheter assembly according to example 139, wherein the planetary gearbox comprises an elongated central gear, wherein the elongated central gear is coupled to the paddle width adjustment element through a rotationally fixed follower such that rotation of the elongated central gear causes axial movement of the rotationally fixed follower, which in turn causes axial movement of the paddle width adjustment element with respect to the housing.

Example 141. The device/implant catheter assembly according to example 140 or example 140, wherein external teeth of the elongated central gear engage with teeth of the pair of planet gears.

Example 142. A handle assembly for controlling a device, the handle assembly comprising: (i) a handle housing; (ii) a sheath extending distally from the handle housing; (iii) an actuation element extending through at least a portion of the sheath, the actuation element configured to be coupled to the device; (iv) a width adjustment element extending through at least a portion of the sheath, the width adjustment element configured to be coupled to at least one of a pair of anchors of the device; (v) an actuation control coupled to the actuation element, wherein actuation of the actuation control causes movement of the actuation element with respect to the handle housing and/or the sheath, wherein the movement of the actuation element can move the device between an open configuration and a closed configuration; and (vi) a width control coupled to the width adjustment element, wherein actuation of the width control causes movement of the width adjustment element relative to the handle housing and/or the sheath, wherein the movement of the width adjustment element can transition at least one of the pair of anchors of the device between a first width and a second width.

Example 143. The handle assembly according to example 142, further comprising one or more clasp actuation lines extending through the sheath, the one or more clasp actuation lines configured to be coupled to the device.

Example 144. The handle assembly according to example 143, wherein the one or more clasp actuation lines include a braided body having a proximal end and a distal end opposite the proximal end, and a closed loop is formed in the distal end.

Example 145. The handle assembly according to any one of examples 142-144, further comprising one or more clasp control members, wherein the one or more clasp control members are movable relative to the handle housing, wherein movement of the one or more clasp control members causes one or more clasps of the device to be moved between an open configuration and a closed configuration.

Example 146. The handle assembly according to any one of examples 143-145, wherein the one or more clasp actuation lines are coupled to a suture lock extending from a proximal end of the handle housing.

Example 147. The handle assembly according to example 146, wherein each suture lock is angled with respect to a central axis extending through the handle assembly.

Example 148. The handle assembly according to any one of examples 142-147, wherein the width control is coupled to a planetary gearbox and actuation of the width control is effective to cause rotation of the planetary gearbox.

Example 149. The handle assembly according to example 148, wherein the planetary gearbox comprises at least a ring gear, a pair of planet gears, and an elongated central gear.

Example 150. The handle assembly according to example 149, wherein the elongated central gear is coupled to the width adjustment element through a rotationally fixed follower such that rotation of the elongated central gear causes axial movement of the rotationally fixed follower, which in turn causes axial movement of the width adjustment element with respect to the housing.

Example 151. The handle assembly according to example 149 or example 150, wherein external teeth of the elongated central gear engage with teeth of the pair of planet gears.

Example 152. A handle assembly for controlling a transvascular device having a plurality of clasps for securing native leaflets of a heart valve, the handle assembly comprising: (i) a handle housing; (ii) a sheath extending distally from the handle housing; (iii) a first clasp actuation element extending through the sheath, the first clasp actuation element operatively coupled to a first clasp of the plurality of clasps on the device; (iv) a first clasp control member operatively connected to the first clasp actuation element, the first clasp control member movable relative to the housing between a first position associated with the first clasp being in an open position and a second position associated with the first clasp being in a closed position; and (v) a first biasing element configured to apply a first force to pull the first clasp toward the open position.

Example 153. The handle assembly according to example 152, wherein the first biasing element applies the first force onto the first clasp actuation element.

Example 154. The handle assembly according to example 153, wherein the first biasing element directly contacts the first clasp actuation element when applying the first force.

Example 155. The handle assembly according to any of examples 153-154, wherein the first force is directed radially outward from a centerline of the handle housing.

Example 156. The handle assembly according to any of examples 153-155, wherein the first force is not applied to pull the first clasp toward the open position when the first clasp control member is in the second position.

Example 157. The handle assembly according to any of examples 152-156, wherein the first biasing element has an elongated body having a proximal end fixed relative to the handle housing and a free distal end.

Example 158. The handle assembly according to example 157, wherein the elongated body has a semi-elliptical shape.

Example 159. The handle assembly according to example 157 or example 158, wherein the first biasing element has an opening extending laterally through the elongated body and the first clasp actuation element extends through the opening.

Example 160. The handle assembly according to example 159, wherein the opening is positioned closer to the distal end than the proximal end.

Example 161. The handle assembly according to any of examples 152-160, wherein the first clasp actuation element is a suture line.

Example 162. The handle assembly according to any of examples 152-161, wherein the first biasing element has a wide position in which the force is applied to pull the first clasp toward the open position and a narrow position in which the force is not applied to pull the first clasp toward the open position.

Example 163. The handle assembly according to example 162, wherein the first biasing element is biased to the wide position.

Example 164. The handle assembly according to example 163, wherein the first clasp control member holds the first biasing element in the narrow position when the first clasp control member is in the second position.

Example 165. The handle assembly according to example 163 or 164, wherein the first clasp control member releases the first biasing element to the wide position when the first clasp control member is in the first position.

Example 166. The handle assembly according to example 152, wherein the first biasing element is positioned in-line with the first clasp actuation element.

Example 167. The handle assembly according to example 166, wherein the first biasing element comprises an elastic portion of the first clasp actuation element.

Example 168. The handle assembly according to example 167, wherein the elastic portion extends along an entire length of the first clasp actuation element.

Example 169. The handle assembly according to example 167, wherein the elastic portion extends along a partial length of the first clasp actuation element.

Example 170. The handle assembly according to example 169, wherein the elastic portion of the first clasp actuation element is positioned inside the handle housing.

Example 171. The handle assembly according to any of examples 166-170, wherein the first biasing element does not apply the first force to pull the first clasp toward the open position when the first clasp control member is in the second position.

Example 172. The handle assembly according to any of examples 152-171, wherein the first clasp control member is movable axially relative to the housing between the first position and the second position.

Example 173. The handle assembly according to any of example 152-172, further comprising: (i) a second clasp actuation element extending through the sheath, the second clasp actuation element operatively coupled to a second clasp of the plurality of clasps on the device; (ii) a second clasp control member operatively connected to the second clasp actuation element, the second clasp control member movable relative to the housing between a third position associated with the second clasp being in an open position and a fourth position associated with the second clasp being in a closed position; and (iii) a second biasing element configured to apply a second force to pull the second clasp toward the open position.

Example 174. The handle assembly according to example 173, wherein the second biasing element is configured to apply the second force independent of the first biasing element.

Example 175. A method of using a treatment device or valve repair device having one or more clasps for securing one or more native leaflets of a heart valve, the method comprising: (i) delivering the treatment device or valve repair device to the heart valve via a device/implant catheter assembly; (ii) moving a first clasp control member to a first position to hold a first clasp of the treatment device or valve repair device in an open position with a first clasp actuation element; (iii) moving the first clasp control member to a second position to close the first clasp of the treatment device or valve repair device to grasp a first leaflet of the heart valve; and wherein holding the first clasp of the treatment device or valve repair device in an open position further comprises applying a first force to the first clasp actuation element after the first clasp control member is in the first position.

Example 176. The method according to example 175, wherein applying the first force to the first clasp actuation element further comprises applying a force onto the first clasp actuation element.

Example 177. The method according to example 175 or example 176, wherein the first force is a radially outward force.

Example 178. The method according to any of examples 175-177, wherein the first clasp actuation element is a suture line.

Example 179. The method according to any of example 175-177, wherein applying the first force to the first clasp actuation element after the first clasp control member is in the first position further comprises moving a biasing element to a wide position.

Example 180. The method according to any of example 179, wherein moving the biasing element to the wide position further comprises moving the first clasp control member to the first position.

Example 181. The method according to example 177 or example 180, wherein moving the first clasp control member to a second position further comprises moving the biasing element to a narrow position.

Example 182. The method according to example 181, wherein moving the biasing element to the narrow position further comprises engaging the biasing element with the first clasp control member.

Example 183. The method according to example 182, further comprising holding the biasing element in the narrow position with the first clasp control member.

Example 184. The method according to any one of examples 175-183, wherein moving the first clasp control member to the second position further comprises moving the first clasp control member axially.

Example 185. The method according to example 175, wherein applying force to the first clasp actuation element further comprises stretching an elastic portion of the first clasp actuation element.

Example 186. The method according to any of examples 175-185, further comprising: (i) moving a second clasp control member to a third position to hold a second clasp of the treatment device or valve repair device in an open position with a second clasp actuation element; (ii) moving the second clasp control member to a fourth position to close the second clasp of the treatment device or valve repair device to grasp a second leaflet of the heart valve; and wherein holding the second clasp of the treatment device or valve repair device in an open position further comprises applying a second force to the second clasp actuation element after the second clasp control member is in the third position.

Example 187. The method according to example 186, wherein the first force is applied independent of the second force.

Example 188. A delivery system for a device having one or more clasps for securing native leaflets of a heart valve, the delivery system comprising a device/implant catheter assembly having a handle assembly for controlling the device and a sheath extending from the handle assembly in an axial direction; wherein the sheath has distal end portion comprising a capture mechanism for releasably attaching the sheath to the device; and wherein the device/implant catheter assembly, comprises: (i) a handle housing, the sheath extending distally from the handle housing; (ii) a first clasp actuation element extending through the sheath, the first clasp actuation element operatively coupled to a first clasp of one or more clasps on the device; (iii) a first clasp control member operatively connected to the first clasp actuation element, the first clasp control member movable relative to the housing between a first position associated with the first clasp being in an open position and a second position associated with the first clasp being in a closed position; and (iv) a first biasing element configured to apply a force to pull the first clasp toward the open position.

Example 189. The delivery system according to example 188, wherein the first biasing element applies the force onto the first clasp actuation element.

Example 190. The delivery system according to example 189, wherein the first biasing element directly contacts the first clasp actuation element when applying the force.

Example 191. The delivery system according to any of examples 188-190, wherein the force is directed radially outward from a centerline of the handle housing.

Example 192. The delivery system according to any of examples 188-191, wherein the force is not applied to pull the first clasp toward the open position when the first clasp control member is in the second position.

Example 193. The delivery system according to any of examples 188-191, wherein the first biasing element has an elongated body having a proximal end fixed relative to the handle housing and a free distal end.

Example 194. The delivery system according to example 193, wherein the elongated body has a semi-elliptical shape.

Example 195. The delivery system according to example 193 or example 194, wherein the first biasing element has an opening extending laterally through the elongated body and the first clasp actuation element extends through the opening.

Example 196. The delivery system according to example 195, wherein the opening is positioned closer to the distal end than the proximal end.

Example 197. The delivery system according to any of examples 188-196, wherein the first clasp actuation element is a suture line.

Example 198. The delivery system according to any of examples 188-197, wherein the first biasing element has a wide position in which the force is applied to pull the first clasp toward the open position and a narrow position in which the force is not applied to pull the first clasp toward the open position.

Example 199. The delivery system according to example 198, wherein the first biasing element is biased to the wide position.

Example 200. The delivery system according to example 199, wherein the first clasp control member holds the first biasing element in the narrow position when the first clasp control member is in the second position.

Example 201. The delivery system according to example 199 or example 200, wherein the first clasp control member releases the first biasing element to the wide position when the first clasp control member is in the first position.

Example 202. The delivery system according to example 188, wherein the first biasing element is positioned in-line with the first clasp actuation element.

Example 203. The delivery system according to example 202, wherein the first biasing element comprises an elastic portion of the first clasp actuation element.

Example 204. The delivery system according to example 203, wherein the elastic portion extends along an entire length of the first clasp actuation element.

Example 205. The delivery system according to example 203, wherein the elastic portion extends along a partial length of the first clasp actuation element.

Example 206. The delivery system according to example 205, wherein the elastic portion of the first clasp actuation element is positioned inside the handle housing.

Example 207. The delivery system according to any of examples 202-206, wherein the first biasing element does not apply the force to pull the first clasp toward the open position when the first clasp control member is in the second position.

Example 208. The delivery system according to any of examples 188-207, wherein the first clasp control member is movable axially relative to the housing between the first position and the second position.

Example 209. The delivery system according to any of examples 188-208, further comprising: (i) a second clasp actuation element extending through the sheath, the second clasp actuation element operatively coupled to a second clasp of the one or more clasps on the device; (ii) a second clasp control member operatively connected to the second clasp actuation element, the second clasp control member movable relative to the housing between a third position associated with the second clasp being in an open position and a fourth position associated with the second clasp being in a closed position; and (iii) a second biasing element configured to apply a second force to pull the second clasp toward the open position.

Example 210. The delivery system according to example 209, wherein the second biasing element is configured to apply the second force independent of the first biasing element.

Example 211. A clasp actuation line for actuating a clasp of a treatment device or valve repair device via a clasp control member, the clasp actuation line comprising: (i) a braided body having a first end configured to operatively couple to the clasp control member and a second end opposite the first end; and (ii) wherein the braided body has a first portion having a first elasticity and a second portion having a second elasticity greater than the first elasticity.

Example 212. The clasp actuation line according to example 211, wherein the first portion of the braided body has a first number of picks per inch and the second portion of the braided body has a second number of picks per inch that is greater than the first number of picks per inch.

Example 213. The clasp actuation line according to example 211, wherein both the first portion and the second portion are formed from an ultra-high-molecular-weight polyethylene material.

Example 214. The clasp actuation line according to example 211, wherein the second portion extends the majority of an entire length of the clasp actuation line.

Example 215. The clasp actuation line according to example 211, wherein the second portion is adjacent the first end and extends less than half a total length of the clasp actuation line.

Example 216. The clasp actuation line according to any one of examples 211-215 used as the clasp actuation line of any of the foregoing systems and/or assemblies of any of the foregoing examples.

Any of the various systems, assemblies, devices, apparatuses, elements, etc. in this disclosure, including the enumerated examples above, can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

While various inventive aspects, concepts and features of the disclosures can be described and illustrated herein as embodied in combination in the example implementations, these various aspects, concepts, and features can be used in many alternative implementations, either individually or in various combinations and sub-combinations thereof. Unless expressly excluded herein all such combinations and sub-combinations are intended to be within the scope of the present application. Still further, while various alternative implementations as to the various aspects, concepts, and features of the disclosures-such as alternative materials, structures, configurations, methods, devices, and components, alternatives as to form, fit, and function, and so on—may be described herein, such descriptions are not intended to be a complete or exhaustive list of available alternative implementations, whether presently known or later developed. Those skilled in the art may readily adopt one or more of the inventive aspects, concepts, or features into additional implementations and uses within the scope of the present application even if such implementations are not expressly disclosed herein.

Additionally, even though some features, concepts, or aspects of the disclosures can be described herein as being a preferred arrangement or method, such description is not intended to suggest that such feature is required or necessary unless expressly so stated. Still further, example or representative values and ranges can be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. Further, the techniques, methods, operations, steps, etc. described or suggested herein can be performed on a living subject (e.g., human, other animal, etc.) or on a non-living subject (e.g., a simulation, such as a cadaver, cadaver heart, simulator, anthropomorphic phantom, etc.). When performed on a simulation, the body parts, e.g., heart, tissue, valve, etc., can optionally be referred to as “simulated” (e.g., simulated heart, simulated tissue, simulated valve, etc.) and can comprise computerized and/or physical representations of the body parts, tissue, etc. The words used in the claims have their full ordinary meanings and are not limited in any way by the description of the implementations in the specification.

What is claimed is:

1. A handle assembly for controlling a transvascular device having a plurality of clasps for securing native leaflets of a heart valve, the handle assembly comprising:
a handle housing;
a sheath extending distally from the handle housing;
a first clasp actuation element extending through the sheath, the first clasp actuation element operatively coupled to a first clasp of the plurality of clasps on the device;
a first clasp control member operatively connected to the first clasp actuation element, the first clasp control member movable relative to the handle housing between a first position associated with the first clasp being in an open position and a second position associated with the first clasp being in a closed position; and
a first biasing element configured to apply a first force to pull the first clasp toward the open position.

2. The handle assembly of claim 1 further comprising:
an actuation element extending through the sheath, the actuation element configured to be coupled to the device;
a paddle actuation control operatively coupled to the actuation element, wherein actuation of the paddle actuation control causes movement of the actuation element with respect to the handle housing and the sheath, wherein the movement of the actuation element causes the device to move between open and closed positions;
a paddle width adjustment element extending through the actuation element, the paddle width adjustment element configured to be coupled to at least one of a pair of paddles of the device; and
a paddle width control operatively coupled to the paddle width adjustment element, wherein actuation of the paddle width control causes movement of the paddle width adjustment element with respect to the handle housing and the sheath, wherein the movement of the paddle width adjustment element causes a width of the at least one of the pair of paddles of the device to be moved from a first width to a second width.

3. The handle assembly according to claim 2, further comprising a release control operatively coupled to the paddle width adjustment element, wherein actuation of the release control causes movement of the paddle width adjustment element and the actuation element to decouple the device from the sheath.

4. The handle assembly according to claim 2, wherein the paddle actuation control is operatively coupled to a retractor to cause the movement of the actuation element, and the paddle width control is operatively coupled to a drive member to cause the movement of the paddle width adjustment element, wherein actuation of the paddle actuation control causes movement of both the retractor and the drive member.

5. The handle assembly of claim 1 further comprising:
an actuation element extending through the sheath, the actuation element configured to be coupled to the device;
a paddle actuation control operatively coupled to the actuation element, wherein actuation of the paddle actuation control causes movement of the actuation element with respect to the handle housing and the sheath, wherein movement of the actuation element causes the device to move between open and closed positions;
a paddle width adjustment element extending through the actuation element, the paddle width adjustment element configured to be coupled to at least one of a pair of paddles of the device; and
a paddle width control operatively coupled to the paddle width adjustment element, wherein actuation of the paddle width control causes movement of the paddle width adjustment element with respect to the handle housing and the sheath, wherein movement of the paddle width adjustment element causes a width of the at least one of the pair of paddles of the device to be moved from a first width to a second width.

6. The handle assembly of claim 1 further comprising a second biasing element that biases the first clasp control member toward the second position.

7. The handle assembly of claim 1 further comprising:
an actuation element extending through the sheath, the actuation element configured to be coupled to the device;
a paddle width adjustment element extending through the actuation element, the paddle width adjustment element configured to be coupled to at least one of a pair of paddles of the device;
a paddle actuation control coupled to the actuation element, wherein actuation of the paddle actuation control causes movement of the actuation element with respect to the handle housing and the sheath, wherein the movement of the actuation element causes the device to be moved between an open configuration and a closed configuration;
a paddle width control coupled to the paddle width adjustment element, wherein actuation of the paddle width control causes movement of the paddle width adjustment element with respect to the handle housing and the sheath, wherein the movement of the paddle width adjustment element causes a width of the at least one of the pair of paddles of the device to be moved from a first width to a second width;
a pair of clasp actuation lines extending through the sheath, each clasp actuation line of the pair of clasp actuation lines configured to be coupled to the device; and
a pair of clasp control members, wherein each clasp control member of the pair of clasp control members is movable relative to the handle housing, wherein the movement of each clasp control member causes a clasp of the device to be moved between an open configuration and a closed configuration.

8. The handle assembly of claim 1 further comprising:
an actuation element extending through at least a portion of the sheath, the actuation element configured to be coupled to the device;
a width adjustment element extending through at least a portion of the sheath, the width adjustment element configured to be coupled to at least one of a pair of anchors of the device;
an actuation control coupled to the actuation element, wherein actuation of the actuation control causes movement of the actuation element with respect to the handle housing and/or the sheath, wherein the movement of the actuation element can move the device between an open configuration and a closed configuration; and
a width control coupled to the width adjustment element, wherein actuation of the width control causes movement of the width adjustment element relative to the handle housing and/or the sheath, wherein the movement of the width adjustment element can transition at least one of the pair of anchors of the device between a first width and a second width.

9. A delivery system for a device having a plurality of clasps for securing native leaflets of a heart valve, the delivery system comprising:
a catheter assembly having a handle assembly for controlling the device and a sheath extending from the handle assembly in an axial direction;
wherein the sheath has distal end portion comprising a capture mechanism for releasably attaching the sheath to the device;
wherein the handle assembly, comprises:
a handle housing;
a proximal end portion of the sheath extending distally from the handle housing;
a first clasp actuation element extending through the sheath, the first clasp actuation element operatively coupled to a first clasp of the plurality of clasps on the device;
a first clasp control member operatively connected to the first clasp actuation element, the first clasp control member movable relative to the housing between a first position associated with the first clasp being in an open position and a second position associated with the first clasp being in a closed position; and
a first biasing element configured to apply a force to pull the first clasp toward the open position.

10. The delivery system of claim 9, wherein the first biasing element comprises a clasp actuation line comprising:
a braided body having a first end configured to operatively couple to the first clasp control member and a second end opposite the first end; and
wherein the braided body has a first portion having a first elasticity and a second portion having a second elasticity greater than the first elasticity.

11. The delivery system of claim 9, wherein:
the distal end portion of the sheath comprising the capture mechanism configured to move between a coupled position in which the capture mechanism is secured to the device and a release position in which the capture mechanism is decoupled from the device;
the first clasp control member comprises a first clasp actuation line, the first clasp actuation line extending from the handle housing, through the sheath, and through a first aperture in the first clasp; and
wherein a first distal end of the first clasp actuation line is attached to the capture mechanism when the capture mechanism is in the coupled position.

12. A delivery system for a device having a plurality of clasps for securing native leaflets of a heart valve, the delivery system comprising:
(i) a device/implant catheter assembly having a handle and a sheath extending from the handle in an axial direction, the sheath having a distal end portion comprising a capture mechanism for attaching the sheath to the device, the capture mechanism configured to move between a coupled position in which the capture mechanism is secured to the device and a release position in which the capture mechanism is decoupled from the device; and
(ii) a first clasp actuation line configured to move a first clasp of the plurality of clasps between a closed position and an open position, the first clasp actuation line extending from the handle, through the sheath, and through a first aperture in the first clasp; and wherein a first distal end of the first clasp actuation line is attached to the capture mechanism when the capture mechanism is in the coupled position.

13. The delivery system according to claim 12, wherein the first distal end of the first clasp actuation line is captured between the capture mechanism and the device when the capture mechanism is in the coupled position.

14. The delivery system according to claim 12, wherein the capture mechanism includes a first longitudinally projecting finger and wherein the first distal end of the first clasp actuation line is attached to the first longitudinally projecting finger.

15. The delivery system according to claim 14, wherein the first distal end of the first clasp actuation line is formed as a first closed loop and the first longitudinally projecting finger extends through the first closed loop.

16. The delivery system according to claim 12, wherein movement of the capture mechanism from the coupled position to the release position and axial movement of the capture mechanism away from the device releases the first distal end of the first clasp actuation line from the capture mechanism.

17. The delivery system according to any of claims 12-16, wherein the handle includes a first clasp control member that engages the first clasp actuation line and is movable relative to the housing, wherein the first clasp control member includes a first passage through which the first clasp actuation line extends, wherein the first clasp actuation line slides through the first passage when the first clasp control member moves relative to the housing.

18. The delivery system of claim 12, wherein the first clasp actuation line comprises an elongate body having a proximal end, a distal end opposite the proximal end, and a closed loop formed at the distal end.

19. The delivery system of claim 12, wherein the first clasp actuation line is coupled to a suture lock extending from a proximal end of the handle.

20. The delivery system of claim 12, wherein the first clasp actuation line is formed by braiding a plurality of a bifurcated portions separated by unitary portions and the first clasp actuation line comprises a section cut to form a single bifurcated portion adjacent a distal end of the section.
